SEQUENCE LISTING (4 sequences)

SEQ ID NO: 1 - DNA, 41 bp, Artificial Sequence
  Primer
catatgaagcttgtcagtgtttgctgcaaatactccccatg

SEQ ID NO: 2 - DNA, 37 bp, Artificial Sequence
  Primer
catatgaagcttcaccgttctcattcacatgatatac

SEQ ID NO: 3 - DNA, 50 bp, Artificial Sequence
  Primer
tgaaatcgccccgaaagactagtagtaagttgtgttgatgccacttcagc

SEQ ID NO: 4 - DNA, 50 bp, Artificial Sequence
  Primer
gctgaagtggcatcaacacaacttactactagtctttcggggcgatttca

(19) United States
(12) Patent Application Publication
(10) Pub. No.: US 2017/0305899 A1
(43) Pub. Date: Oct. 26, 2017

(54) ANTIBIOTIC COMPOUNDS AND COMPOSITIONS, AND METHODS FOR IDENTIFICATION THEREOF

(71) Applicants: Fred Hutchinson Cancer Research Center, Seattle, WA (US); The Scripps Research Institute, La Jolla, CA (US)

(72) Inventors: Gerald R. Smith, Seattle, WA (US); Susan K. Amundsen, Seattle, WA (US); Ahmet C. Karabulut, Seattle, WA (US); Thomas D. Bannister, Palm Beach Gardens, FL (US); Reji Narayanan Nair, Palm Beach Gardens, FL (US)

(21) Appl. No.: 15/342,056

(22) Filed: Nov. 2, 2016

Related U.S. Application Data
(63) Continuation of application No. 14/386,298, filed on Sep. 18, 2014, now abandoned.
(60) Provisional application No. 61/613,367, filed on Mar. 20, 2012.

(51) Int. Cl. C07D 471/04; A61K 31/497; A61K 31/5365; C12Q 1/44; A61K 31/495; C12Q 1/18; A61K 31/519

(52) U.S. Cl. CPC C07D 471/04; C12Q 1/18; A61K 31/497; A61K 31/519; C12Q 1/44; A61K 31/495; A61K 31/5365; G01N 2333/922; G01N 2500/04; G01N 2500/10

(57) ABSTRACT

Disclosed herein are compounds and methods for inhibiting bacterial DNA repair enzymes, including AddAB and RecBCD helicase-nucleases. Pharmaceutical compositions and methods for treating a subject with an antibacterial agent are also disclosed herein.

### CROSS-REFERENCE TO RELATED APPLICATIONS

This application is a Continuation of U.S. application Ser. No. 14/386,298, filed Sep. 18, 2014, which is a U.S. National Stage Application of PCT/US2013/033195, filed Mar. 20, 2013, which claims the benefit under 35 U.S.C. §119(e) of U.S. Application No. 61/613,367, filed on Mar. 20, 2012, all of which are incorporated by reference in their entirety.

### ACKNOWLEDGEMENT OF GOVERNMENT SUPPORT

This invention was made with government support under GM031693, AI083736, and MH084512 awarded by the National Institutes of Health. The government has certain rights in the invention.

### TECHNICAL FIELD

The invention relates to compounds and compositions that inhibit bacterial DNA helicase, nuclease, or helicase-nuclease complex enzymes. In certain embodiments, in addition to inhibiting bacterial helicase, compounds and compositions described herein exhibit a dual functionality and inhibit bacterial DNA gyrase enzymes as well. Methods for identifying and using compounds and compositions described herein are also provided.

### BRIEF DESCRIPTION OF THE DRAWINGS

FIG. 1 describes the general principle of the cell-based screen for AddAB and RecBCD inhibitors, according to an embodiment of the present disclosure. Left: Activities of RecBCD and AddAB helicase-nucleases. Both enzymes (open circle) are active on linear duplex DNA (double lines). ds Exonuclease activity involves a combination of ATP-dependent DNA unwinding and endonucleolytic cuts. ss DNA intermediates are digested to short TCA-soluble oligonucleotides by the ss exonuclease activity. Right: RecBCD or AddAB nuclease activity blocks the growth of phage T4 gene 2 mutants. Upon injection into E. coli cells, wild-type T4 DNA is protected from AddAB and RecBCD nucleases by the gene 2 protein bound to the linear duplex DNA ends in the virion; phages grow and the cells are killed. Unprotected T4 gene 2 mutant DNA is digested by the nucleases; cells grow. Inhibition of AddAB or RecBCD is detected by lack of cell growth after T4 gene 2 mutant infection.

FIG. 2 describes how T4 gene 2 mutant phage prevent the growth of E. coli lacking RecBCD enzyme but not growth of wild type, according to an embodiment of the present disclosure. E. coli strain V66 (recBCD⁺, open symbols) and strain V67 (recB21, closed symbols) were infected with T4 gene 2 triple non-nonsense mutant phage (MOI=0.01; circles) or not (squares). Cultures were 0.1 ml in a 96-well plate, which was shaken at 37° C. in an incubated plate reader. Each data point is the mean of 24 wells; SEM is within the size of the symbols. Similar results were found with E. coli expressing H. pylori AddAB.

FIG. 3 shows general structures of five classes of AddAB and RecBCD inhibitors identified by screening, according to various embodiments of the present disclosure.

FIG. 4 describes the inhibition of H. pylori AddAB and E. coli RecBCD nuclease activities by various compounds identified in the primary screen, according to embodiments of the present disclosure. ds Exonuclease activity of AddAB (filled circles) and RecBCD (open squares) was measured in the presence of the indicated concentration of compound and expressed as a percent of the activity in the absence of compound. Curves were fit by GraphPad software using the four-parameter logistic nonlinear regression model. For Compounds 1 and 4, the data are means+/−SEM with n=3 or 4; for the other compounds, data are from one experiment.

FIG. 5 describes the inhibition of E. coli RecBCD unwinding and Chi cutting activities by various compounds identified in the primary screen, according to embodiments of the present disclosure. DNA unwinding and cutting at Chi hotspots by RecBCD enzyme was assayed in the presence of the indicated concentration of compound. Unwinding is indicated by the amount of ss DNA and Chi cutting by the amount of Chi-dependent 1.46 kb ss DNA fragment (“Chi”) produced from the 4.36 kb ds DNA substrate.

FIG. 6 describes the inhibition of H. pylori AddAB and E. coli RecBCD nuclease activities by derivatives of Compounds 1 and 4, according to embodiments of the present disclosure. ds Exonuclease activity was measured in the presence of the indicated concentration of compound and expressed as a percent of the activity in the absence of compound.

FIG. 7 describes the inhibition of E. coli Hfr recombination by selected compounds, according to embodiments of the present disclosure. The frequency of His⁺ Str^{R} recombinants in matings between strains V66 (F⁻ recBCD⁺ hisG4 rpsL31) and V1306 (Hfr P044 rpsL⁺ his⁺) in the presence of compound (the concentration in μM as indicated for Compound 7 applies to all compounds) is expressed as a fraction of that in the absence of compound (9.3% per viable Hfr cell). Data are from one experiment; similar results were obtained in two others.

FIG. 8 describes the inhibition of phage λ recombination by selected compounds, according to embodiments of the present disclosure. The mean frequency of J⁺ R⁺ recombinants in λ crosses (1081×1082 and 1083×1084) in strain V66 in the presence of the indicated compound (concentration in μM as indicated for Compound 2 applies to all compounds) is expressed as a fraction of that without compound (6.9±0.25%; n=4).

FIG. 9 describes the inhibition of E. coli RecBCD nuclease, unwinding, and Chi cutting activities by compounds of structural class E, according to embodiments of the present disclosure. ds Exonuclease activity (left panel) and unwinding and Chi cutting activities (right panel) were measured in the presence of the indicated concentration of compound. ds Exonuclease activity is expressed as a percent of the activity in the absence of compound. Unwinding is indicated by the amount of ss DNA, and Chi cutting by the amount of Chi-dependent ss DNA fragment (“Chi”).

FIG. 10 shows that the AddAB DNA unwinding activity is not altered by certain compounds, according to embodiments of the present disclosure. DNA unwinding by AddAB enzyme was assayed in the presence of compound (50 μM). Unwinding is indicated by the amount of ss DNA (heavy arrow).

FIG. 11 shows a screen for active derivatives of Compounds 1 and 4, according to embodiments of the present disclosure. E. coli strain V66 (recBCD⁺) or strain V67 (recB21, RecBCD⁻) (left panels) or strain V3065 (addAB⁺) or strain V3069 (empty vector control; AddAB⁻) (right panels) in the presence of the indicated compound (50 μM) were infected with T4 gene 2 triple nonsense mutant (grey bar; MOI=0.01) or not (black bar), and the optical density measured after ˜20 h of incubation. Data are the mean and SEM of 4 wells in 2 independent experiments.

FIG. 12 describes the inhibition of H. pylori AddAB and E. coli RecBCD nuclease activities by derivatives of Compounds 1 and 4, according to embodiments of the present disclosure. ds Exonuclease activity was measured in the presence of the indicated concentration of compound and expressed as a percent of the activity in the absence of compound. A separate experiment with RecBCD and 50 μM of Compound 1 derivatives showed the same pattern.

FIG. 13 describes the inhibition of E. coli Hfr recombination by derivatives of Compounds 1 and 4, according to embodiments of the present disclosure. The frequency of His⁺ Str^{R} recombinants in matings between strains V66 (F⁻ recBCD⁺ hisG4 rpsL31) and V1306 (Hfr P044 rpsL⁺ his⁺) in the presence of compound is expressed as a fraction of that in the absence of compound (4.4% per viable Hfr cell).

FIG. 14 describes the inhibition of E. coli RecBCD nuclease activity by compounds of structural class E, according to embodiments of the present disclosure. ds Exonuclease activity was measured in the presence of compound (100 μM) and expressed as a percent of the activity in the absence of compound.

FIG. 15 shows the minimum concentration of Compound 50 and norfloxacin required to inhibit the growth of E. coli strain V66 (recBCD⁺) as measured by optical density. In triplicate, 100 μl of cells from an actively growing culture with the indicated titer (cfu/ml) were seeded into a 96-well plate. Compound was added to the indicated concentration, and the plate was incubated at 37° C. for 18 hours. The reported optical densities are the means of 3-well sets. The MIC of Compound 50 is 1 μM, and that of norfloxacin 0.25 μM. “Uninoc” represents an uninoculated control.

FIG. 16 shows the inhibition of E. coli growth by Compound 50 or norfloxacin. An overnight culture of strain V66 (recBCD⁺) was diluted to 1×10⁶ cells per ml and added to wells of a microtiter plate. After 1 hour of incubation at 37° C., norfloxacin or Compound 50 was added to the concentration indicated. The optical density was measured at the times indicated. Each data point represents the mean optical density of 3 wells; the values differed by less than 1%. At the end of the incubation period the number of viable cells in each 3-well pool was determined. Except for the untreated control (1.4×10⁹ colony forming units per ml), all cultures contained <200 colony forming units per ml.

FIG. 17 shows that Compound 50 inhibits E. coli recombination in an Hfr cross, as measured by the relative frequency of His⁺ Str^{R} recombinants in a cross between strain V66 (hisG4 rpsL31) and V1306 (Hfr P044 rpsL⁺ hisG⁺). Cells were treated with the indicated concentration of compound for 45 minutes prior to the cross, made by mixing V66 and V1306 at a ratio of 10:1, incubating 30 min, vortexing, and plating for His⁺ Str^{R} recombinants. Data are from a single experiment and are expressed relative to the untreated control, which had 12.4% recombinants per viable Hfr donor. The viability of V66 in the presence and absence of compound was indistinguishable.

FIG. 18 shows a comparison of Compounds 1, 2, 50 and 51 in their inhibition of E. coli RecBCD ds exonuclease activity assayed as in FIG. 12. Compounds 1, 50, and 51 inhibit more strongly than norfloxacin.

FIG. 19 shows the effect of Compound 1 on the ciprofloxacin sensitization of an E. coli V66 wild type strain.

FIG. 20 shows a dose response study of Compound 1 in the inhibition of E. coli RecBCD helicase unwinding activity.

FIG. 21 shows a dose response study of Compound 50 in the inhibition of E. coli RecBCD and H. pylori AddAB nucleases.

FIG. 22 shows a dose response study of the inhibition of E. coli RecBCD DNA unwinding activity and Chi cutting activity for Compound 50.

FIG. 23 shows a dose response study of Compound 151 and Compound 148 for purified Mycobacterium tuberculosis AddAB enzyme.

FIG. 24 shows the results of an E. coli precA::lacZ reporter assay for the measurement of SOS induction by norfloxacin.

FIG. 25 shows the results of an E. coli precA::lacZ reporter assay for the measurement of SOS induction by H₂O₂ with or without compound 151.

FIG. 26 shows the effects of AddAB inhibitors on the ability of Helicobacter pylori to colonize the stomach of mice.

FIG. 27 shows the effects of RecBCD inhibitor compound 3 on the frequency of H₂O₂-induced mutation in E. coli strain V66.

FIG. 28 shows the effects of RecBCD inhibitor compound 3 on the frequency of an H₂O₂-induced mutation to valine-resistance (valine^{R}) in E. coli strain V66. Data are the mean±SEM (N=16).

FIG. 29 shows one embodiment of a method for the synthesis of compound 3.

### DETAILED DESCRIPTION

Disclosed herein are compounds and compositions useful as inhibitors of bacterial helicase-nuclease DNA repair enzymes. Compounds and compositions exhibiting a dual functionality are also described. In particular, in certain embodiments, the compounds and compositions detailed herein inhibit both bacterial DNA helicase-nuclease and bacterial DNA gyrase enzymes. Methods for identifying compounds and compositions according to the present description are also provided. Moreover, because DNA helicase-nuclease and DNA gyrase functionality are important to bacterial infection in mammals, methods of inhibiting or treating bacterial infection are also disclosed.

### I. Definitions

It is to be understood that the terminology used herein is for the purpose of describing particular embodiments only, and is not intended to limit the scope of the present invention which will be limited only by the appended claims.

Unless defined otherwise, all technical and scientific terms used herein have the meanings that would be commonly understood by one of skill in the art. As used herein, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 50%, 30%, 20%, 10%, 5%, 1%, 0.5%, or even 0.1% of the specified amount. All publications, patent applications, patents, and other references mentioned herein are incorporated by reference in their entirety.

It will be appreciated that the compounds, as described herein, may be substituted with substituents or functional moieties as described herein. When more than one position in any given structure may be substituted with more than one substituent selected from a specified group, the substituent may be either the same or different at every position. As used herein, the term “substituted” is contemplated to include all permissible substituents of organic compounds, including but not limited to acyclic and cyclic, branched and unbranched, carbocyclic and heterocyclic, aromatic and nonaromatic substituents of organic compounds. For purposes of the present disclosure, heteroatoms such as nitrogen may have hydrogen substituents and/or any permissible substituents of organic compounds described herein which satisfy the valencies of the heteroatoms.

“Alkyl” as used herein alone or as part of another group, refers to a straight, branched and/or cyclic hydrocarbon containing from 1 to 10 carbon atoms. In some embodiments, the alkyl employed in the invention contains 1 to 6 carbon atoms. Representative examples of alkyl include, but are not limited to, methyl, ethyl, n-propyl, iso-propyl, cyclopropyl, n-butyl, sec-butyl, iso-butyl, tert-butyl, n-pentyl, isopentyl, neopentyl, n-hexyl, cyclohexyl, 3-methylhexyl, 2,2-dimethylpentyl, 2,3-dimethylpentyl, n-heptyl, n-octyl, n-nonyl, and n-decyl.

“Lower alkyl” as used herein, is a subset of alkyl, in some embodiments preferred, and refers to a straight, branched and/or cyclic hydrocarbon group containing from 1 to 4 carbon atoms. Representative examples of lower alkyl include, but are not limited to, methyl, ethyl, n-propyl, iso-propyl, cyclopropyl, n-butyl, iso-butyl, and tert-butyl.

The term “alkyl” or “lower alkyl” is intended to include both substituted and unsubstituted alkyl or lower alkyl unless otherwise indicated and these groups may be substituted with groups selected from halo (e.g., haloalkyl), alkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, aryl thioamido, haloalkylaryl thioamido, aryl amido, haloalkylaryl amido mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano, where m=0, 1, 2 or 3.

“Alkenyl” as used herein alone or as part of another group, refers to a straight, branched and/or cyclic chain hydrocarbon containing from 1 to 10 carbon atoms (or in lower alkenyl 1 to 4 carbon atoms) which include 1 to 4 double bonds in the normal chain. In some embodiments, the alkenyl employed in the invention contains 1 to 6 carbon atoms. Representative examples of alkenyl include, but are not limited to, vinyl, 2-propenyl, 3-butenyl, 2-butenyl, 4-pentenyl, 3-pentenyl, 2-hexenyl, 3-hexenyl, 2,4-heptadiene, and the like. The term “alkenyl” or “lower alkenyl” is intended to include both substituted and unsubstituted alkenyl and lower alkenyl unless otherwise indicated and these groups may be substituted with groups as described in connection with alkyl and lower alkyl above. Cycloalkenyl refers to a cyclic alkenyl group.

“Alkynyl” as used herein alone or as part of another group, refers to a straight or branched chain hydrocarbon containing from 1 to 10 carbon atoms (or in lower alkynyl 1 to 4 carbon atoms) which include 1 to 4 triple bonds in the normal chain. In some embodiments, the alkynyl employed in the invention contain 1 to 6 carbon atoms. Representative examples of alkynyl include, but are not limited to, 2-propynyl, 3-butynyl, 2-butynyl, 4-pentynyl, and 3-pentynyl. The term “alkynyl” or “lower alkynyl” is intended to include both substituted and unsubstituted alkynyl and lower alkynyl unless otherwise indicated and these groups may be substituted with the same groups as set forth in connection with alkyl and lower alkyl above.

“Cycloalkyl”, as used herein alone or as part of another group, refers to groups having 3 to 10 carbon atoms. In some embodiments, the cycloalkyl employed in the invention has 3 to 8 carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with the same groups as set forth in connection with alkyl and lower alkyl above. The term “alkyl” includes cycloalkyl. The cycloalkyl may be a bicycloalkyl.

“Heterocycloalkyl” or “heterocycle”, as used herein alone or as part of another group, refers to a non-aromatic 3-, 4-, 5-, 6-, 7-, or 8-membered ring or a polycyclic group, including, but not limited to a bi- or tri-cyclic group comprising fused six-membered rings having between one and four heteroatoms independently selected from oxygen, sulfur and nitrogen, wherein (i) the nitrogen and sulfur heteroatoms may be optionally oxidized, (ii) the nitrogen heteroatom may optionally be quaternized, and (iii) may form a spiro ring or be fused with a cycloalkyl, aryl, heterocyclic ring, benzene or a heteroaromatic ring. In some embodiments, the heterocycle employed in the invention has 3 to 10 carbon atoms. For example, the heterocycle may be a 4-(2-halophenylcarbamothioyl)piperazin-1-yl.

Representative heterocycles include, but are not limited to, 1,4-dioxa-8-azaspiro[4,5]decane, morpholine, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazolone, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, benzimidazole, benzothiazole, benzothiadiazole, benzothiophene, benzoxadiazole, benzoxazole, benzofliran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, and thiopyranopyridine. These rings include quaternized derivatives thereof and may be optionally substituted with the same groups as set forth in connection with alkyl and lower alkyl above.

“Aryl” as used herein alone or as part of another group, refers to a monocyclic carbocyclic ring system or a bicyclic carbocyclic fused ring system having one or more aromatic rings. In some embodiments, the aryl employed in the invention has 3 to 14 carbon atoms.

Representative examples of aryl include azulenyl, indanyl, indenyl, naphthyl, phenyl, and tetrahydronaphthyl. The term “aryl” is intended to include both substituted and unsubstituted aryl unless otherwise indicated, and these groups may be optionally substituted with the same groups as set forth in connection with alkyl and lower alkyl above.

“Aryl alkyl” as used herein alone or as part of another groups refers to an aryl group, as defined herein, appended to the parent molecular moiety through an alkyl group, as defined herein. Representative examples of aryl alkyl include, but are not limited to, benzyl, 2-phenyl ethyl, 3-phenylpropyl, and 2-naphth-2-ylethyl.

“Heteroaryl” as used herein alone or as part of another group, refers to a cyclic or bicyclic, aromatic hydrocarbon in which one or more carbon atoms have been replaced with heteroatoms such as 0, N, and S. If the heteroaryl group contains more than one heteroatom, the heteroatoms may be the same or different. In some embodiments, the heteroaryl employed in the invention have 3 to 14 carbon atoms. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, quinolinyl, phthalazinyl, naphthyridinyl, quinoxalinyl, isothiazolyl, and benzo[b]thienyl. In some embodiments, heteroaryl groups are five and six membered rings and contain from one to three heteroatoms independently selected from O, N, and S. The heteroaryl group, including each heteroatom, can be unsubstituted or substituted with from 1 to 4 substituents, as chemically feasible. For example, the heteroatom N or S may be substituted with one or two oxo groups, which may be shown as ═O. For example, the heteroaryl group may be a 4-oxo-1,4-dihydroquinoline-3-carboxylic acid.

“Alkoxy” (or “alkyloxy”), or “thioalkyl”, as used herein alone or as part of another group, refers to an alkyl or lower alkyl group appended to the parent molecular moiety through an oxygen or sulfur atom. In some embodiments, the alkoxy or thioalkyl group contains 1-10 carbon atoms. In other embodiments, the alkyl, alkenyl, and alkynyl groups employed in the invention contain 1-8 carbon atoms. In still other embodiments, the alkyl group contains 1-6 carbon atoms. In yet other embodiments, the alkyl group contains 1-4 carbon atoms. Representative examples, of alkoxy, include but are not limited to, methoxy, ethoxy, propoxy, isopropoxy, n-butoxy, tert-butoxy, neopentoxy and n-hexoxy. Representative examples of thioalkyl include, but are not limited to, methylthio, ethylthio, propylthio, isopropylthio, and n-butylthio.

“Halo” as used herein alone or as part of another group, refers to any suitable halogen, including —F, —Cl, —Br, and —I.

“Amine” or “amino group”, as used herein alone or as part of another group, refers to the radical —NH₂. An “optionally substituted” amine refers to —NH₂ groups wherein none, one or two of the hydrogen(s) is replaced by a suitable substituent. Disubstituted amines may have substituents that are bridging, i.e., that form a heterocyclic ring structure that includes the amine nitrogen.

“Aminoalkyl group” is intended to mean the radical —NHR₃, where R₃ is an alkyl group.

“Haloalkyl”, as used herein alone or as part of another group, refers to an alkyl group having one, two, or three halogen atoms attached thereto and is exemplified by such groups as chloromethyl, bromoethyl, and trifluoromethyl.

The terms “therapeutically useful” and “therapeutically effective” refer to a dose or amount of a compound or composition that causes a detectable change in biological or chemical activity, such as a detectable change in the inhibition of a bacterial helicase (e.g., a RecBCD and/or AddAB enzyme), a bacterial gyrase, and/or in bacterial growth. The terms “therapeutically useful” and “therapeutically effective” can designate an amount that maintains a desired physiological state, i.e., reduces or prevents significant decline and/or promotes improvement in the condition or disease of interest. For example, a therapeutically effective or useful amount of a compound or composition described herein would be an amount that inhibits, slows or eliminates growth of bacteria in a subject. As is generally understood in the art, therapeutically effective dosages will vary depending on the administration routes, symptoms and body weight of the patient but also depending upon the compound being administered.

The terms “active” or “biologically active” or “biological activity” refer to a compound or composition capable of inhibiting a bacterial helicase (e.g., RecBCD and/or AddAB activity, either the helicase or the nuclease activity or a combination of both) and/or a bacterial gyrase, to affect growth of a bacterium, such as E. coli. In some embodiments, an active or biologically active compound or composition as described herein as may have an IC₅₀ of less than about 100 micromol/liter (100 μM), less than about 50 micromol/liter (50 μM), less than about 10 micromol/liter (10 μM), or less than about 1 micromol/liter (1 μM). As used herein, the IC₅₀ is the concentration (μM) of compound or composition that results in 50% inhibition of enzyme activity (e.g. for RecBCD, AddAB, or bacterial gyrase activity) or cell growth or other cellular activity (e.g. for bacterial viability or recombination studies).

Further, an active or biologically active agent or composition as described herein may be alternatively or additionally characterized as an agent or composition that that inhibits growth of bacteria in a subject. For example, an active or biologically active agent or composition as described herein may be characterized as an agent or composition that causes a greater than 2-fold change, greater than 5-fold change, greater than 10-fold change, greater than 15-fold change, and greater than 20-fold change in bacterial growth, as compared to growth free from the agent.

In further embodiments, an active or biologically active agent, compound, or composition as described herein may be characterized as an agent, compound, or composition that selectively inhibits a bacterial helicase, such as RecBCD and/or AddAB. In other embodiments, an active or biologically active agent, compound, or composition as described herein may be characterized as an agent, compound, or composition that selectively inhibits a bacterial gyrase. In still further embodiments, an active or biologically active agent, compound, or composition as described herein may be characterized as a dual function agent, compound, or composition that selectively inhibits both a bacterial helicase, such as RecBCD and/or AddAB, and a bacterial gyrase. For example, an active or biologically active agent or compound or composition as described herein may have a selective inhibition greater than about 2-fold, about 5-fold, about 10-fold, about 15-fold, and about 20-fold of bacterial helicase, such as RecBCD and/or AddAB, and/or a bacterial gyrase where the bacteria is present in a subject. In such examples, an active or biologically active agent or compound or composition as described herein may be characterized as an agent or composition that is substantially non-toxic to the subjects' cells, such as mammalian cells.

For purposes of the present disclosure, the terms “antibiotic” and/or “antibacterial” includes diseases, disorders, and conditions that are linked to the presence of at least some bacteria in a subject. Such diseases include, but are not limited to, community or nosocomial acquired infections, bacteremias, bacterium-related cutaneous, gastrointestinal and respiratory conditions, botulism, cholera, E. coli infection, Legionellosis, listeriosis, Lyme disease, pathogenic bacterial diseases, rickttsioses, salmonellosis, tuberculosis and zoonotic bacterial diseases.

Bacteria which may be affected by compounds and compositions disclosed herein include, for example, bacterial infections by both Gram-positive and Gram-negative bacteria, such as Escherichia coli, Enterobacter cloacae, Klebsiella pneumoniae, Morganella morganii, Salmonella serotypes including Enteritidis, Typhimurium and Newport, Enterococci, Shigella dysenteriae, Yersinia enterocolitica, Acinetobacter calcoaceticus, Francisella tularensis, Legionella pneumophila, Helicobacter pylori, Neisseria meningitides, Neisseria gonorrhoeae, Campylobacter jejuni, Vibrio cholera, Pseudomonas aeruginosa, Streptococcus, Staphylococcus, pneumococcus, Mycobacterium tuberculosis, Borrelia burgdorferi, Bordetella pertussis, Legionella pneumophila, Clostridium difficile, Bacillus anthracis, and Haemophilus influenzae.

Bacteria-associated diseases further include those which involve antibacterial drug resistance, such as Methicillin-resistant Staphylococcus aureus (MRSA) infection.

The term “pharmaceutically acceptable” refers to materials approved by a regulatory agency, such as by a regulatory agency of a Federal or a state government, or listed in the U.S. Pharmacopeia or other generally recognized pharmacopeia for use in animals, including humans.

In the context of the present description, the term “carrier” refers to a diluent, adjuvant, excipient, stabilizer, vehicle, or any combination thereof, with which an active compound as described herein may be combined to provide a pharmaceutical composition suitable for administration to a subject.

The term “subject” refers to refers to an animal, including humans, in which an active compound as described herein will be therapeutically useful (e.g., selectively inhibit bacterial growth). The subject may be a veterinary subject, including birds and livestock.

The term “pharmaceutically acceptable salt(s)” as used herein refers to a salt form of a compound permitting its use or formulation as a pharmaceutical and which retains the biological effectiveness of the free acid and base of the specified compound and that is not biologically or otherwise undesirable. Examples of such salts are described in Handbook of Pharmaceutical Salts: Properties, Selection, and Use, Wermuth, C. G. and Stahl, P. H. (eds.), Wiley-Verlag Helvetica Acta, Zurich, 2002.

Examples of pharmaceutically acceptable salts, without limitation, include those formed with free amino groups such as those derived from hydrochloric, phosphoric, acetic, oxalic, or tartaric acids, and those formed with free carboxyl groups such as those derived from sodium, potassium, ammonium, calcium, ferric hydroxides, isopropylamine, triethylamine, 2-ethylamino ethanol, histidine, or procaine. Examples of salts also include sulfates, pyrosulfates, bisulfates, sulfites, bisulfites, phosphates, monohydrogenphosphates, dihydrogen phosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, di nitrobenzoates, hydroxybenzoates, methoxybenzoates, phthalates, xylenesulfonates, phenylacetates, phenylpropionates, phenylbutyrates, citrates, lactates, γ-hydroxybutyrates, glycollates, tartrates, methanesulfonates, ethane sulfonates, propanesulfonates, toluenesulfonates, naphthalene-1-sulfonates, naphthalene-2-sulfonates, and mandelates. In some embodiments, a pharmaceutically acceptable salt includes sodium, potassium, calcium, ammonium, trialkylarylammonium and tetraalkylammonium salts.

As used herein, “treatment” of a disease, disorder, or infection refers to, but is not limited to, prevention, retardation and prophylaxis of the disease, disorder, or infection.

### II. Compounds

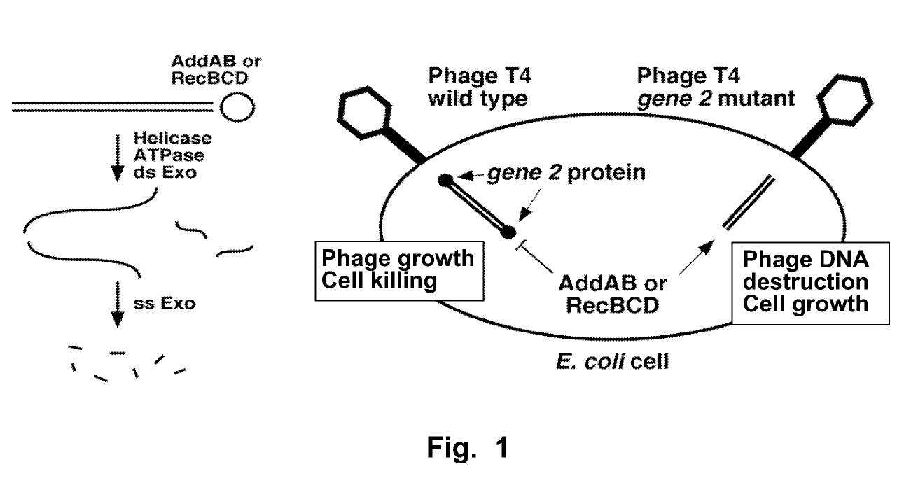

The compounds described herein may be single function (i.e., inhibit one or more bacterial DNA helicase, nuclease, or helicase-nuclease complex or one or more bacterial DNA gyrase) or dual function (i.e., inhibit both one or more bacterial DNA helicase, nuclease, or helicase-nuclease complex while also inhibiting bacterial DNA gyrase). In specific embodiments, the compounds inhibit helicase enzymes selected from one or both of the RecBCD and AddAB families of helicase-nucleases. The AddAB and RecBCD helicase-nucleases are related enzymes prevalent among bacteria but not eukaryotes, and are instrumental in the repair of DNA double-strand breaks and in genetic recombination. The RecBCD class of enzymes and the closely related AddAB enzymes are bacterial helicase-nuclease complexes important for repair of broken DNA and for genetic recombination. Dillingham, M. S.; Kowalczykowski, S. C., Microbiol Mol Biol Rev 2008, 72 (4), 642-71; Smith, G. R., Annu. Rev. Genet. 2001, 35, 243-274; Smith, G. R., Microbiol Mol Biol Rev 2012, 76, 217-228. Starting at a double-strand (ds) DNA end, these enzymes unwind DNA rapidly and highly processively while hydrolyzing ATP or another nucleoside triphosphate (FIG. 1). During unwinding, they also hydrolyze DNA by making endonucleolytic scissions at a rate dependent on the ratio of [ATP] to [Mg²⁺], both of which are required for the helicase and nuclease activities. The AddAB and RecBCD enzymes are needed for successful bacterial infection of animals including mammals, and compounds and compositions described herein may be used as antibacterial agents. Several structural classes of inhibitors of the three-subunit E. coli RecBCD enzyme and the related two-subunit Helicobacter pylori AddAB enzyme are disclosed herein. Also disclosed herein are inhibitors of Mycobacterium tuberculosis AddAB and RecBCD and inhibitors of Mycobacterium smegmatis AddAB and RecBCD. Moreover, the compounds described herein may be useful in further enzymatic, genetic, and physiological studies of these enzymes, both purified and in cells.

The RecBCD enzyme of Escherichia coli makes endonucleolytic scissions at especially high frequency at Chi sites (5′ GCTGGTGG 3′), which as a consequence are hotspots of recombination. Ponticelli, A. S.; Schultz, D. W.; Taylor, A. F.; Smith, G. R., Cell 1985, 41, 145-151. The RecBCD and AddAB enzymes from other species similarly act at other short nucleotide sequences. Touzain, F.; Petit, M. A.; Schbath, S.; El Karoui, M., Nat Rev Microbiol 2011, 9 (1), 15-26. The single-stranded (ss) DNA resulting from unwinding is a potent substrate for the enzymes' ATP-dependent ss nuclease, which, at least for the RecBCD enzyme of E. coli, produces a limit digest of primarily tetra- to hexanucleotides. Goldmark, P. J.; Linn, S., J. Biol. Chem. 1972, 247, 1849-1860. Because this class of enzymes is important for the repair of DNA double-strand (ds) breaks, mutants lacking them are deficient in infecting mammals. Without being bound by theory, this is likely because mammalian cells produce reactive oxygen species, such as hydrogen peroxide, that break the bacterial DNA upon infection. Buchmeier, N. A.; Lipps, C. J.; So, M. Y.; Heffron, F., Mol Microbiol 1993, 7 (6), 933-6, 7, 933-936; Buchmeier, N. A.; Libby, S. J.; Xu, Y.; Loewen, P. C.; Switala, J.; Guiney, D. G.; Fang, F. C., J. Clin. Invest. 1995, 95 (3), 1047-53; Amundsen, S. K.; Fero, J.; Hansen, L. M.; Cromie, G. A.; Solnick, J. V.; Smith, G. R.; Salama, N. R., Molec. Microb. 2008, 69, 994-1007. Proper function of each subunit of RecBCD is required for normal nuclease activity, and small molecules that bind to or otherwise inhibit the functionality of one or more RecBCD subunits can inhibit the nuclease activity exhibited by the enzyme. In AddAB, each subunit contains a nuclease domain; only AddA appears to have an active helicase domain, but its inactivation blocks all detectable nuclease activity (Kooistra, J.; Haijema, B. J.; Hesseling-Meinders, A.; Venema, G., Molec. Microb. 1997, 23 (1), 137-49; Amundsen, S. K.; Fero, J.; Salama, N. R.; Smith, G. R., J. Biol. Chem. 2009, 284, 16759-66; Haijema, B. J.; Meima, R.; Kooistra, J.; Venema, G., J. Bacteriol. 1996, 178 (17), 5130-7; Sinha, K. M.; Unciuleac, M. C.; Glickman, M. S.; Shuman, S., Genes & Development 2009, 23 (12), 1423-37). Similar to RecBCD, each subunit of AddAB is required for normal nuclease activity, and small molecules that bind to or otherwise inhibit the functionality of one or more AddAB subunits can inhibit the nuclease activity exhibited by the enzyme, either directly or indirectly, and allow T4 gene 2 mutant phage to grow, thereby blocking the growth of E. coli.

In further embodiments, compounds described herein inhibit bacterial DNA gyrase. For example, fluoroquinolone compounds capable of inhibiting DNA gyrase are detailed herein. In still other embodiments, the compounds described herein are capable of inhibiting both bacterial DNA helicase, such as for example a helicase selected from one or both of the RecBCD and AddAB families of helicases, and bacterial DNA gyrase. Such dual function (also referred to herein as “dual mechanism of action”) compounds provide targeted inhibition of both helicase and gyrase enzymes within a single molecular moiety. Dual function compounds as described herein both (i) induce DNA damage through gyrase inhibition and (ii) block the repair of such damage, and thereby may provide significant additional functionality beyond single function actives providing helicase or gyrase inhibition alone. For example, dual function compounds described herein that exhibit activity against bacterial recombination and DNA repair proteins may not only serve as broad spectrum antibiotics, but may also act to combat antibiotic resistance by, for example, reducing the rate of appearance of resistant mutants. Compounds according to the present description may also be co-administered with known antibiotics, such as, for example, known fluoroquinolones or other antibiotics that induce SOS response, as compounds providing a potent inhibitory effect on bacterial DNA helicase(s), such as RecBCD and/or AddAB, would be useful in treating microbial infections as a first line or augmentation therapy for treating both susceptible pathogens and reducing the emergence of drug resistant microorganisms.

In one embodiment, active compounds that inhibit bacterial helicase(s) are selected from a compound of structural class A (the “pyrimidopyridones”), according to Formula I:

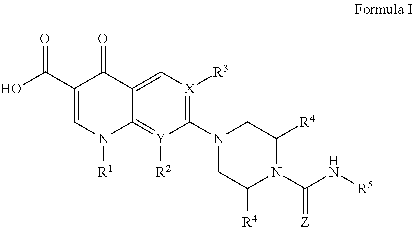

wherein R¹ is alkyl, aryl, or cycloalkyl;
R² is H, alkoxyl or halogen;
R³ is H or halogen;
R⁴ is H or alkyl;
R⁵ is selected from at least one of the following: alkyl, alkenyl, aryl, alkyl aryl, —CO-aryl, —CO-alkyl aryl, cycloalkyl, heteroaryl, and —CO-heteroaryl, any of which may be optionally substituted with a substituent selected from at least one of the following: alkyl, haloalkyl, alkoxy, methylenedioxy, halogen, ethylenedioxy, and nitro;
X and Y are independently C or N; and
Z is O or S.

In particular embodiments of active compounds according to Formula I, R¹ is ethyl, X and Y are each N, R⁴ is H, Z is S and R⁵ is phenyl substituted with a CF₃ group. In an embodiment, the CF₃ group is in the ortho position. In an additional embodiment, the CF₃ group is in the meta position.

In certain embodiments, R¹ is ethyl, X and Y are each C, R² is hydrogen, R³ is halogen, R⁴ is H, Z is S and R⁵ is phenyl substituted with a CF₃ group. In an embodiment, the CF₃ group is in the meta position.

Compounds according to Formula I may be selected to exhibit an inhibitory effect on one or more bacterial DNA helicases, nucleases, or helicase-nuclease enzyme complexes, such as, for example, one or more enzymes selected from the RecBCD and AddAB families of enzymes. Specific examples of compounds according to Formula I include the compounds in Table 1.

TABLE 1
compounds according to Formula I.

| | |
|---|---|
| 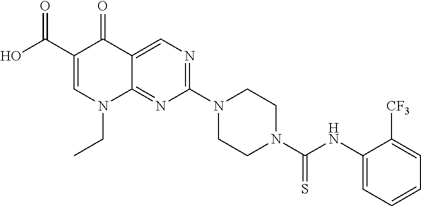 [SMILES: CCn1cc(C(=O)O)c(=O)c2cnc(N3CCN(C(=S)Nc4ccccc4C(F)(F)F)CC3)nc21] | Compound 1 |
| 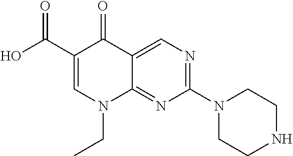 [SMILES: CCn1cc(C(=O)O)c(=O)c2cnc(N3CCNCC3)nc21] | Compound 2 |
| 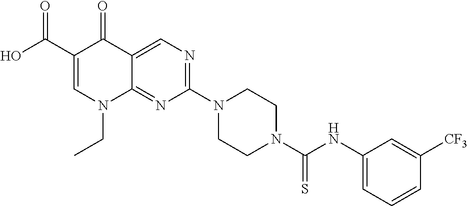 [SMILES: CCn1cc(C(=O)O)c(=O)c2cnc(N3CCN(C(=S)Nc4cccc(C(F)(F)F)c4)CC3)nc21] | Compound 3 |
| 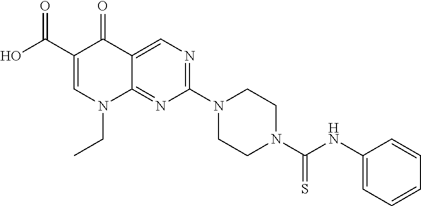 [SMILES: CCn1cc(C(=O)O)c(=O)c2cnc(N3CCN(C(=S)Nc4ccccc4)CC3)nc21] | Compound 19 |
| 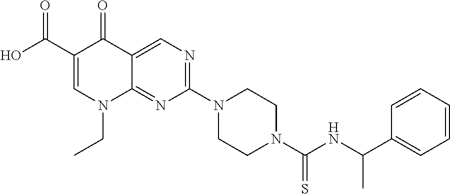 [SMILES: CCn1cc(C(=O)O)c(=O)c2cnc(N3CCN(C(=S)NC(C)c4ccccc4)CC3)nc21] | Compound 20 |
| 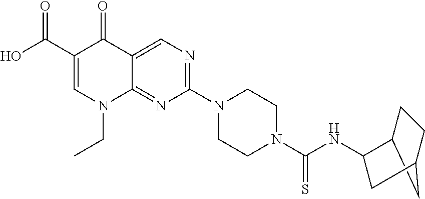 [SMILES: CCn1cc(C(=O)O)c(=O)c2cnc(N3CCN(C(=S)NC4CC5CCC4C5)CC3)nc21] | Compound 21 |
| 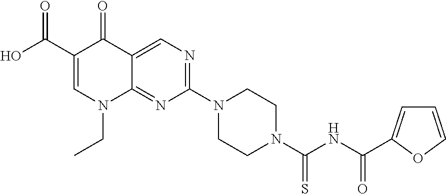 [SMILES: CCn1cc(C(=O)O)c(=O)c2cnc(N3CCN(C(=S)NC(=O)c4ccco4)CC3)nc21] | Compound 22 |
| 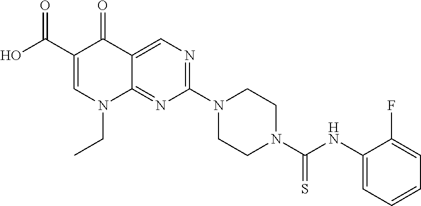 [SMILES: CCn1cc(C(=O)O)c(=O)c2cnc(N3CCN(C(=S)Nc4ccccc4F)CC3)nc21] | Compound 23 |
| 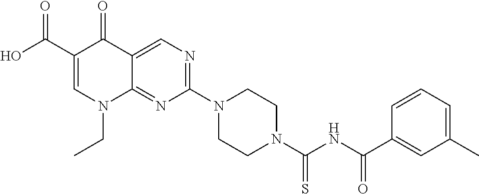 [SMILES: CCn1cc(C(=O)O)c(=O)c2cnc(N3CCN(C(=S)NC(=O)c4cccc(C)c4)CC3)nc21] | Compound 24 |
| 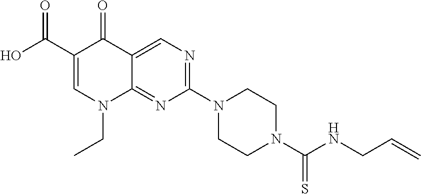 [SMILES: C=CCNC(=S)N1CCN(c2ncc3c(=O)c(C(=O)O)cn(CC)c3n2)CC1] | Compound 25 |
| 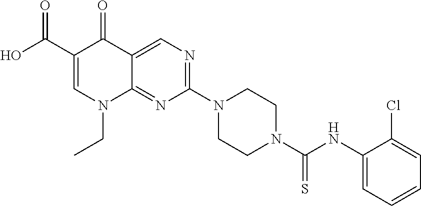 [SMILES: CCn1cc(C(=O)O)c(=O)c2cnc(N3CCN(C(=S)Nc4ccccc4Cl)CC3)nc21] | Compound 26 |
| 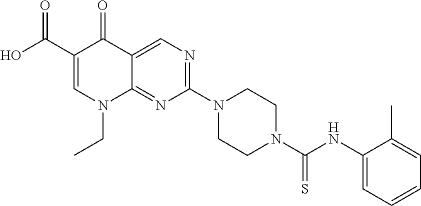 [SMILES: CCn1cc(C(=O)O)c(=O)c2cnc(N3CCN(C(=S)Nc4ccccc4C)CC3)nc21] | Compound 27 |
| 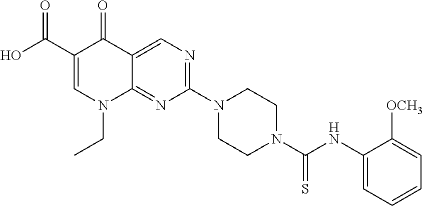 [SMILES: CCn1cc(C(=O)O)c(=O)c2cnc(N3CCN(C(=S)Nc4ccccc4OC)CC3)nc21] | Compound 28 |
| 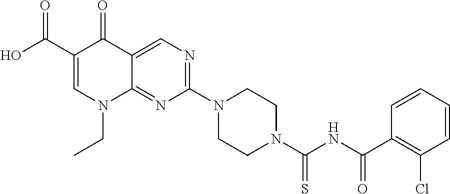 [SMILES: CCn1cc(C(=O)O)c(=O)c2cnc(N3CCN(C(=S)NC(=O)c4ccccc4Cl)CC3)nc21] | Compound 29 |
| 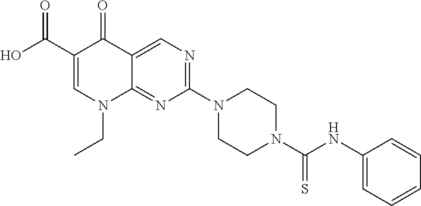 [SMILES: CCn1cc(C(=O)O)c(=O)c2cnc(N3CCN(C(=S)Nc4ccccc4)CC3)nc21] | Compound 30 |
| 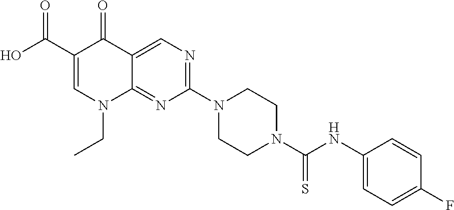 [SMILES: CCn1cc(C(=O)O)c(=O)c2cnc(N3CCN(C(=S)Nc4ccc(F)cc4)CC3)nc21] | Compound 31 |
| 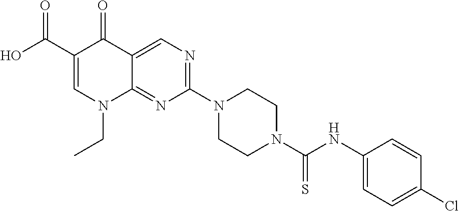 [SMILES: CCn1cc(C(=O)O)c(=O)c2cnc(N3CCN(C(=S)Nc4ccc(Cl)cc4)CC3)nc21] | Compound 32 |
| 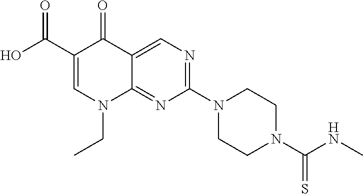 [SMILES: CCn1cc(C(=O)O)c(=O)c2cnc(N3CCN(C(=S)NC)CC3)nc21] | Compound 33 |
| 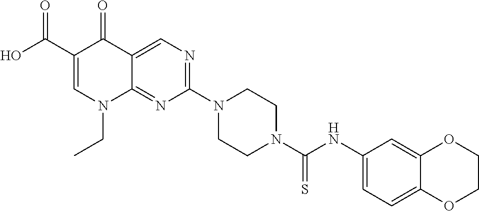 [SMILES: CCn1cc(C(=O)O)c(=O)c2cnc(N3CCN(C(=S)Nc4ccc5c(c4)OCCO5)CC3)nc21] | Compound 34 |
| 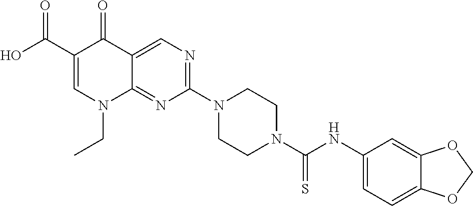 [SMILES: CCn1cc(C(=O)O)c(=O)c2cnc(N3CCN(C(=S)Nc4ccc5c(c4)OCO5)CC3)nc21] | Compound 35 |
| 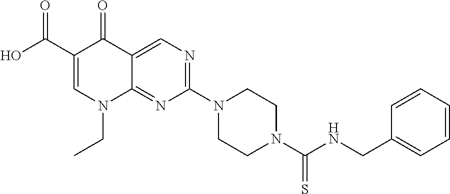 [SMILES: CCn1cc(C(=O)O)c(=O)c2cnc(N3CCN(C(=S)NCc4ccccc4)CC3)nc21] | Compound 36 |
| 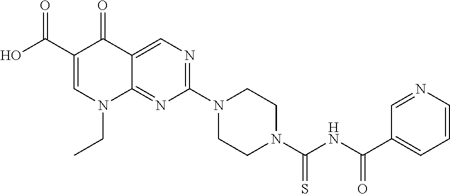 [SMILES: CCn1cc(C(=O)O)c(=O)c2cnc(N3CCN(C(=S)NC(=O)c4cccnc4)CC3)nc21] | Compound 37 |
| 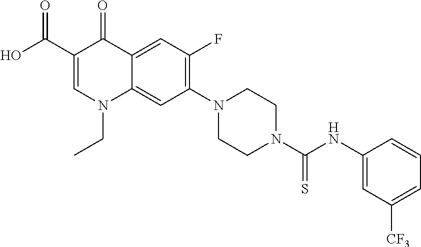 [SMILES: CCn1cc(C(=O)O)c(=O)c2cc(F)c(N3CCN(C(=S)Nc4cccc(C(F)(F)F)c4)CC3)cc21] | Compound 50 |
| 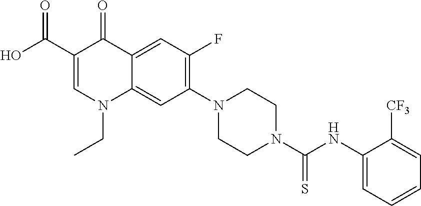 [SMILES: CCn1cc(C(=O)O)c(=O)c2cc(F)c(N3CCN(C(=S)Nc4ccccc4C(F)(F)F)CC3)cc21] | Compound 51 |
| 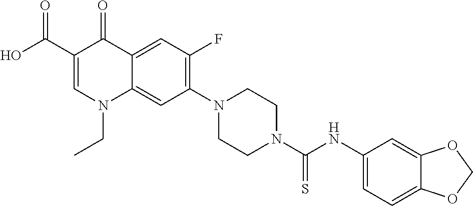 [SMILES: CCn1cc(C(=O)O)c(=O)c2cc(F)c(N3CCN(C(=S)Nc4ccc5c(c4)OCO5)CC3)cc21] | Compound 52 |
| 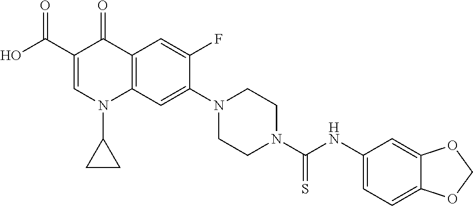 [SMILES: O=C(O)c1cn(C2CC2)c2cc(N3CCN(C(=S)Nc4ccc5c(c4)OCO5)CC3)c(F)cc2c1=O] | Compound 53 |
| 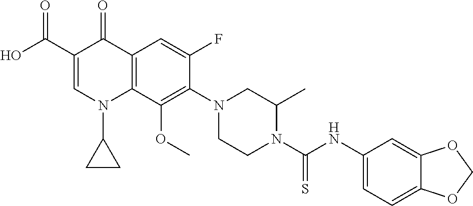 [SMILES: COc1c(N2CCN(C(=S)Nc3ccc4c(c3)OCO4)C(C)C2)c(F)cc2c(=O)c(C(=O)O)cn(C3CC3)c12] | Compound 54 |
| 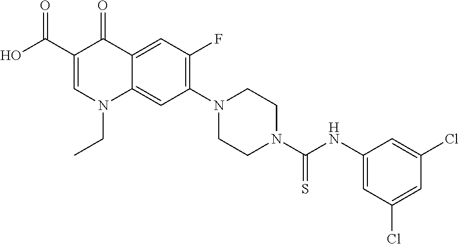 [SMILES: CCn1cc(C(=O)O)c(=O)c2cc(F)c(N3CCN(C(=S)Nc4cc(Cl)cc(Cl)c4)CC3)cc21] | Compound 55 |
| 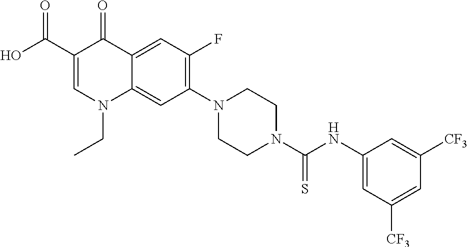 [SMILES: CCn1cc(C(=O)O)c(=O)c2cc(F)c(N3CCN(C(=S)Nc4cc(C(F)(F)F)cc(C(F)(F)F)c4)CC3)cc21] | Compound 56 |
| 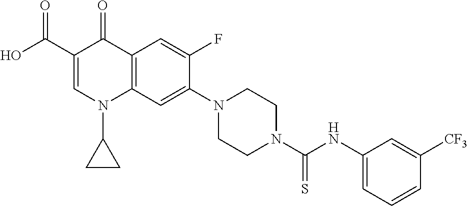 [SMILES: O=C(O)c1cn(C2CC2)c2cc(N3CCN(C(=S)Nc4cccc(C(F)(F)F)c4)CC3)c(F)cc2c1=O] | Compound 57 |
| 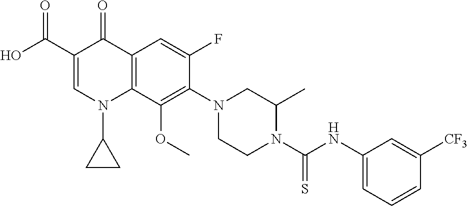 [SMILES: COc1c(N2CCN(C(=S)Nc3cccc(C(F)(F)F)c3)C(C)C2)c(F)cc2c(=O)c(C(=O)O)cn(C3CC3)c12] | Compound 58 |
| 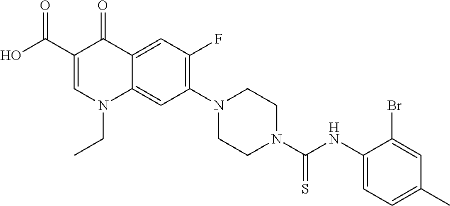 [SMILES: CCn1cc(C(=O)O)c(=O)c2cc(F)c(N3CCN(C(=S)Nc4ccc(C)cc4Br)CC3)cc21] | Compound 59 |
| 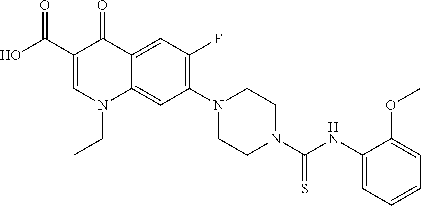 [SMILES: CCn1cc(C(=O)O)c(=O)c2cc(F)c(N3CCN(C(=S)Nc4ccccc4OC)CC3)cc21] | Compound 60 |
| 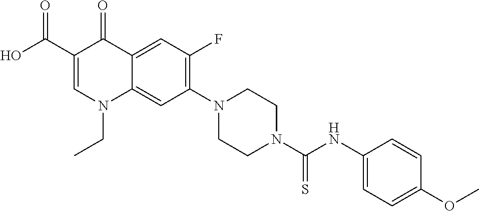 [SMILES: CCn1cc(C(=O)O)c(=O)c2cc(F)c(N3CCN(C(=S)Nc4ccc(OC)cc4)CC3)cc21] | Compound 61 |
| 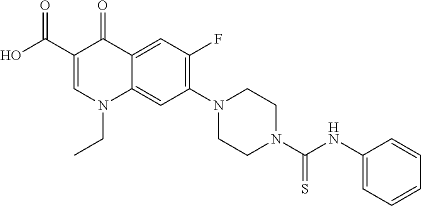 [SMILES: CCn1cc(C(=O)O)c(=O)c2cc(F)c(N3CCN(C(=S)Nc4ccccc4)CC3)cc21] | Compound 62 |
| 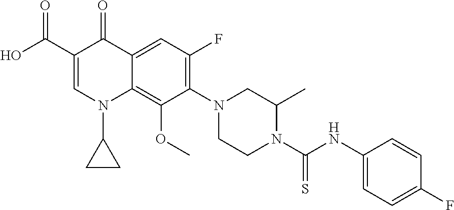 [SMILES: COc1c(N2CCN(C(=S)Nc3ccc(F)cc3)C(C)C2)c(F)cc2c(=O)c(C(=O)O)cn(C3CC3)c12] | Compound 63 |
| 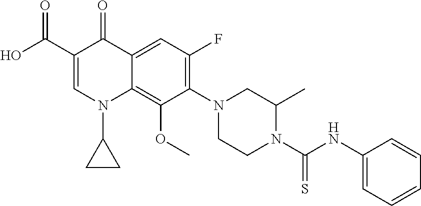 [SMILES: COc1c(N2CCN(C(=S)Nc3ccccc3)C(C)C2)c(F)cc2c(=O)c(C(=O)O)cn(C3CC3)c12] | Compound 64 |
| 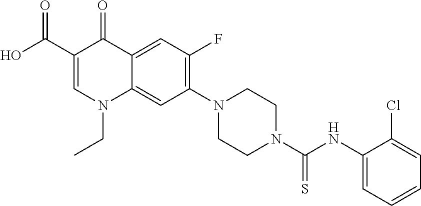 [SMILES: CCn1cc(C(=O)O)c(=O)c2cc(F)c(N3CCN(C(=S)Nc4ccccc4Cl)CC3)cc21] | Compound 65 |
| 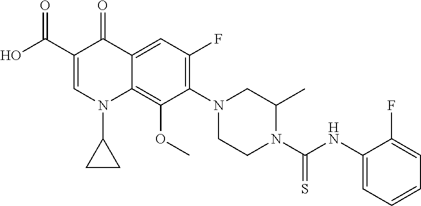 [SMILES: COc1c(N2CCN(C(=S)Nc3ccccc3F)C(C)C2)c(F)cc2c(=O)c(C(=O)O)cn(C3CC3)c12] | Compound 66 |
| 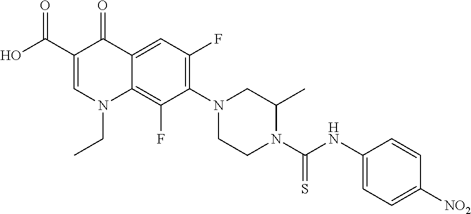 [SMILES: CCn1cc(C(=O)O)c(=O)c2cc(F)c(N3CCN(C(=S)Nc4ccc([N+](=O)[O-])cc4)C(C)C3)c(F)c21] | Compound 67 |
| 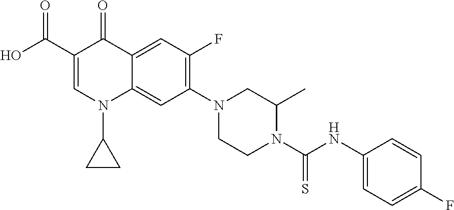 [SMILES: CC1CN(c2cc3c(cc2F)c(=O)c(C(=O)O)cn3C2CC2)CCN1C(=S)Nc1ccc(F)cc1] | Compound 68 |
| 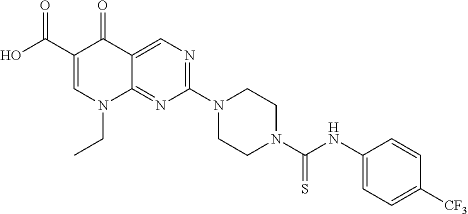 [SMILES: CCn1cc(C(=O)O)c(=O)c2cnc(N3CCN(C(=S)Nc4ccc(C(F)(F)F)cc4)CC3)nc21] | Compound 143 |
| 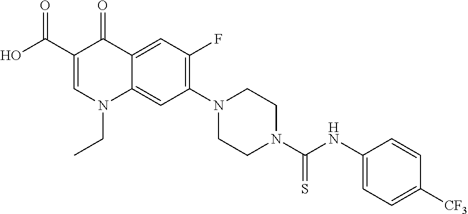 [SMILES: CCn1cc(C(=O)O)c(=O)c2cc(F)c(N3CCN(C(=S)Nc4ccc(C(F)(F)F)cc4)CC3)cc21] | Compound 144 |
| 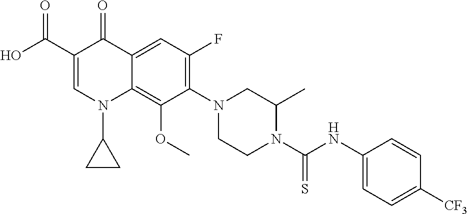 [SMILES: COc1c(N2CCN(C(=S)Nc3ccc(C(F)(F)F)cc3)C(C)C2)c(F)cc2c(=O)c(C(=O)O)cn(C3CC3)c12] | Compound 145 |
| 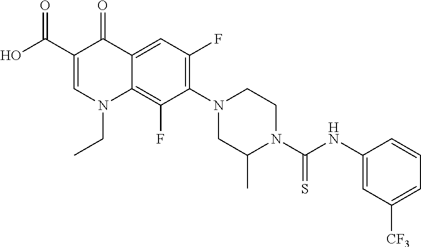 [SMILES: CCn1cc(C(=O)O)c(=O)c2cc(F)c(N3CCN(C(=S)Nc4cccc(C(F)(F)F)c4)C(C)C3)c(F)c21] | Compound 146 |
| 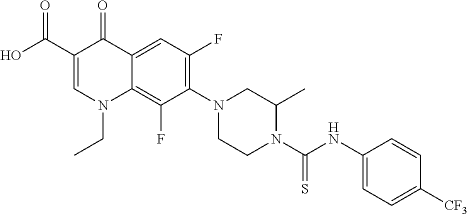 [SMILES: CCn1cc(C(=O)O)c(=O)c2cc(F)c(N3CCN(C(=S)Nc4ccc(C(F)(F)F)cc4)C(C)C3)c(F)c21] | Compound 147 |
| 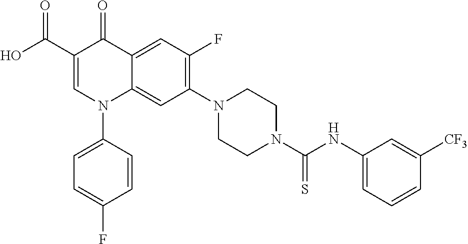 [SMILES: O=C(O)c1cn(-c2ccc(F)cc2)c2cc(N3CCN(C(=S)Nc4cccc(C(F)(F)F)c4)CC3)c(F)cc2c1=O] | Compound 148 |
| 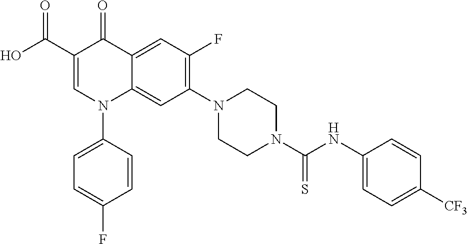 [SMILES: O=C(O)c1cn(-c2ccc(F)cc2)c2cc(N3CCN(C(=S)Nc4ccc(C(F)(F)F)cc4)CC3)c(F)cc2c1=O] | Compound 149 |
| 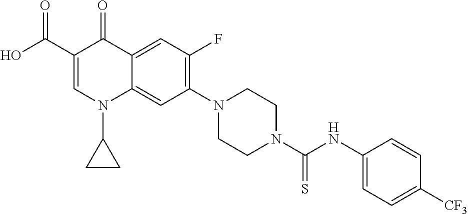 [SMILES: O=C(O)c1cn(C2CC2)c2cc(N3CCN(C(=S)Nc4ccc(C(F)(F)F)cc4)CC3)c(F)cc2c1=O] | Compound 150 |

In certain embodiments, the active compounds as described herein are selected from compounds according to Formula Ia:

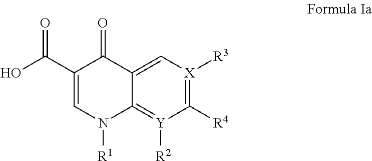

wherein R¹ is alkyl, aryl, or cycloalkyl;
R² is H, alkoxyl or halogen;
R³ is H or halogen;
R⁴ is selected from at least one of the following: alkyl, alkenyl, aryl, alkyl aryl, cycloalkyl, heteroaryl, alkyl heteroaryl, heterocyclyl, and heterocyclyl alkyl, any of which may be optionally substituted; and
X and Y are independently C or N.

In certain embodiments,
R¹ is alkyl, R² is H, R³ is fluorine, X and Y are each C, and R⁴ is

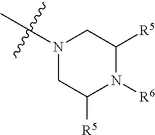

wherein R⁵ is H or alkyl; and
R⁶ is H, —C(═O)NH—R⁷ or —C(═S)NH—R⁷, wherein R⁷ is selected from at least one of the following: alkyl, alkenyl, aryl, alkyl aryl, —CO-aryl, —CO-alkyl aryl, cycloalkyl, heteroaryl, and —CO-heteroaryl, any of which may be optionally substituted with a substituent selected from at least one of the following: alkyl, haloalkyl, alkoxy, methylenedioxy, halogen, ethylenedioxy, and nitro. For example in specific embodiments, R⁷ is —C(═S)NH-phenyl wherein the phenyl is optionally substituted with a haloalkyl group. Examples of such embodiments of compounds of Formula Ia include Compound 50 and Compound 51.

In other embodiments of compounds according to Formula Ia, X and Y are each N, R¹ is alkyl, and R⁴ is

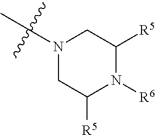

wherein R⁵ is H or alkyl; and
R⁶ is H, —C(═O)NH—R⁷ or —C(═S)NH—R⁷, wherein R⁷ is selected from at least one of the following: alkyl, alkenyl, aryl, alkyl aryl, —CO-aryl, —CO-alkyl aryl, cycloalkyl, heteroaryl, and —CO-heteroaryl, any of which may be optionally substituted with a substituent selected from at least one of the following: alkyl, haloalkyl, alkoxy, methylenedioxy, halogen, ethylenedioxy, and nitro. For example in specific embodiments, R⁷ is —C(═S)NH-phenyl wherein the phenyl is optionally substituted with a haloalkyl group.

In further embodiment of compounds according to Formula Ia, R⁴ may comprise one or more of the R⁴ groups in Table 2.

TABLE 2
Examples of R⁴ groups.

| | |
|---|---|
| 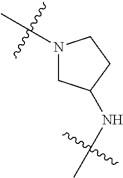 [SMILES: *NC1CCN(*)C1] | Group 1 |
| 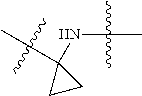 [SMILES: *NC1(*)CC1] | Group 2 |
| 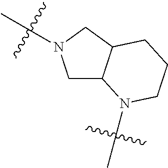 [SMILES: *N1CC2CCCN(*)C2C1] | Group 3 |
| 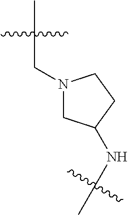 [SMILES: *CN1CCC(N*)C1] | Group 4 |
| 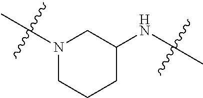 [SMILES: *NC1CCCN(*)C1] | Group 5 |
| 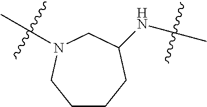 [SMILES: *NC1CCCCN(*)C1] | Group 6 |
| 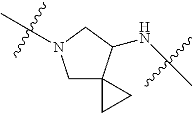 [SMILES: *NC1CN(*)CC12CC2] | Group 7 |
| 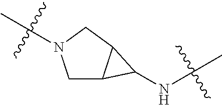 [SMILES: *NC1C2CN(*)CC21] | Group 8 |

wherein each of the R⁴ groups comprises at least two active nitrogen atoms and the R⁴ groups may bond to Formula Ia at R⁴ with one of the active nitrogen atoms. In certain embodiments, the other active nitrogen atom(s) may bound to H or —C(═O)NH—R⁷ or —C(═S)NH—R⁷, wherein R⁷ is selected from at least one of the following: alkyl, alkenyl, aryl, alkyl aryl, —CO-aryl, —CO-alkyl aryl, cycloalkyl, heteroaryl, and —CO-heteroaryl, any of which may be optionally substituted with a substituent selected from at least one of the following: alkyl, haloalkyl, alkoxy, methylenedioxy, halogen, ethylenedioxy, and nitro. For example in specific embodiments, R⁷ is —C(═S)NH-phenyl wherein the phenyl is optionally substituted with a haloalkyl group.

Compounds according to Formula Ia may be selected to exhibit an inhibitory effect on one or more bacterial DNA helicases, nucleases, or helicase-nuclease enzyme complexes, such as, for example, one or more enzymes selected from the RecBCD and AddAB families of enzymes. Specific examples of compounds according to Formula Ia include the compounds in Table 3.

TABLE 3
Compounds according to Formula Ia.

| | |
|---|---|
| 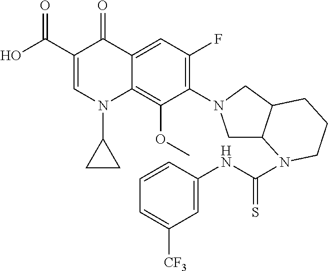 [SMILES: COc1c(N2CC3CCCN(C(=S)Nc4cccc(C(F)(F)F)c4)C3C2)c(F)cc2c(=O)c(C(=O)O)cn(C3CC3)c12] | Compound 151 |
| 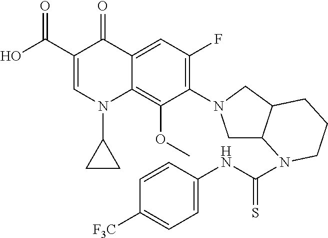 [SMILES: COc1c(N2CC3CCCN(C(=S)Nc4ccc(C(F)(F)F)cc4)C3C2)c(F)cc2c(=O)c(C(=O)O)cn(C3CC3)c12] | Compound 152 |
| 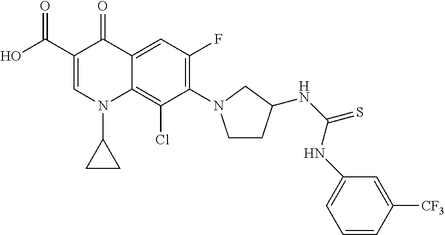 [SMILES: O=C(O)c1cn(C2CC2)c2c(Cl)c(N3CCC(NC(=S)Nc4cccc(C(F)(F)F)c4)C3)c(F)cc2c1=O] | Compound 153 |
| 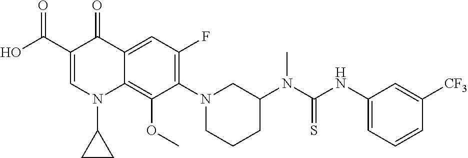 [SMILES: COc1c(N2CCCC(N(C)C(=S)Nc3cccc(C(F)(F)F)c3)C2)c(F)cc2c(=O)c(C(=O)O)cn(C3CC3)c12] | Compound 154 |
| 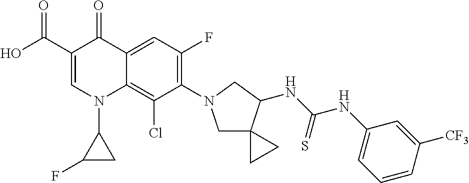 [SMILES: O=C(O)c1cn(C2CC2F)c2c(Cl)c(N3CC(NC(=S)Nc4cccc(C(F)(F)F)c4)C4(CC4)C3)c(F)cc2c1=O] | Compound 155 |
| 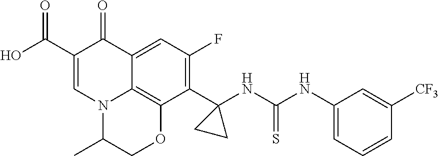 [SMILES: CC1COc2c(C3(NC(=S)Nc4cccc(C(F)(F)F)c4)CC3)c(F)cc3c(=O)c(C(=O)O)cn1c23] | Compound 156 |
| 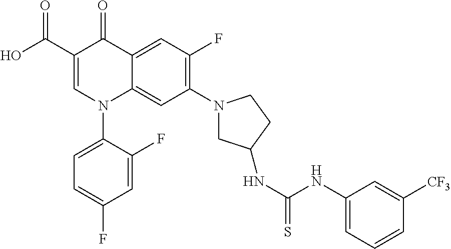 [SMILES: O=C(O)c1cn(-c2ccc(F)cc2F)c2cc(N3CCC(NC(=S)Nc4cccc(C(F)(F)F)c4)C3)c(F)cc2c1=O] | Compound 157 |
| 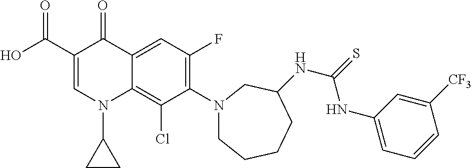 [SMILES: O=C(O)c1cn(C2CC2)c2c(Cl)c(N3CCCCC(NC(=S)Nc4cccc(C(F)(F)F)c4)C3)c(F)cc2c1=O] | Compound 157 |
| 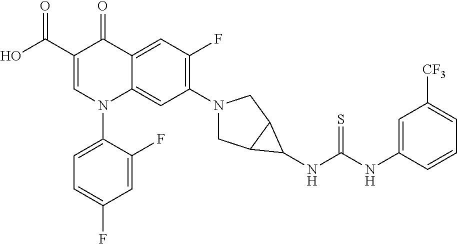 [SMILES: O=C(O)c1cn(-c2ccc(F)cc2F)c2cc(N3CC4C(C3)C4NC(=S)Nc3cccc(C(F)(F)F)c3)c(F)cc2c1=O] | Compound 159 |
| 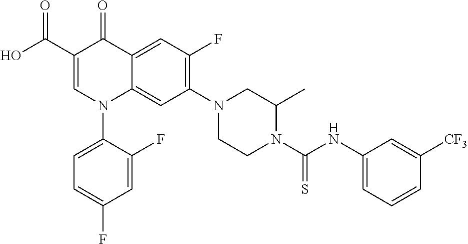 [SMILES: CC1CN(c2cc3c(cc2F)c(=O)c(C(=O)O)cn3-c2ccc(F)cc2F)CCN1C(=S)Nc1cccc(C(F)(F)F)c1] | Compound 160 |

In certain embodiments, the compounds of Formula Ia inhibit bacterial DNA gyrase activity. In additional embodiments, the compounds of Formula Ia inhibit not only bacterial DNA gyrase, but also one or both of the nuclease and the helicase activity of a bacterial helicase, nuclease, or helicase-nuclease complex. For example, in such embodiments, compounds according to Formula Ia may inhibit the nuclease and/or helicase activities of a bacterial nuclease selected from the RecBCD and/or AddAB families of enzymes.

In other embodiments, active compounds as disclosed herein are selected from compounds according to Formula Ib:

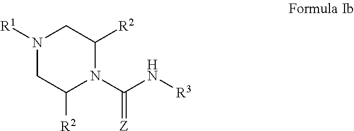

wherein R¹ is is selected from at least one of the following: alkyl, alkenyl, aryl, alkyl aryl, cycloalkyl, heteroaryl, alkyl heteroaryl, heterocyclyl, and heterocyclyl alkyl, any of which may be optionally substituted;
R² is H or alkyl;
R³ is selected from at least one of the following: alkyl, alkenyl, aryl, alkyl aryl, —CO-aryl, —CO-alkyl aryl, cycloalkyl, heteroaryl, and —CO-heteroaryl, any of which may be optionally substituted with a substituent selected from at least one of the following: alkyl, haloalkyl, alkoxy, methylenedioxy, halogen, ethylenedioxy, and nitro; and
Z is O or S.

In particular embodiments of compounds according to Formula Ib, R¹ is selected from a compound according to Formula Ic or Formula Id:

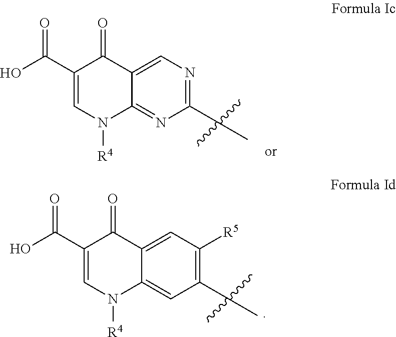

In specific embodiments of compounds according to Formula 1 b, R¹ is selected from a compound according to Formula 10, R² is H, Z is S, and R³ is phenyl substituted with a CF₃ group. In such embodiments, the CF₃ group may be located in any of the ortho, para, or meta positions. Specific examples of such embodiments, include, for example, Compound 1 and Compound 3.

In other embodiments of active compounds according to Formula Ib, R¹ is selected from a compound according to Formula Id, R² is H, Z is S, R³ is phenyl substituted with a CF₃ group, R⁴ is alkyl, and R⁵ is fluorine. In such embodiments, the CF₃ group may be located in any of the ortho, para, or meta position. Specific examples of such embodiments, include, for example, Compound 50 and Compound 51.

In particular embodiments, compounds according to Formula Ib inhibit the helicase and/or the nuclease activity of a bacterial nuclease, helicase, or helicase-nuclease complex. For example, in such embodiments, compounds according to Formula Ib may inhibit the nuclease and/or nuclease activity of a bacterial nuclease selected from the bacterial RecBCD and/or AddAB families of enzymes. In other embodiments, compounds of Formula Ib inhibit the helicase and/or the nuclease activity of a bacterial nuclease, such as, for example the nuclease and/or helicase activity of enzymes selected from one or both of the RecBCD and AddAB families of enzymes, in combination with inhibiting bacterial DNA gyrase activity.

In another embodiment, active compounds that inhibit bacterial helicase(s) are selected from a compound of structural class B (the “cyanothiophenes”), according to Formula II:

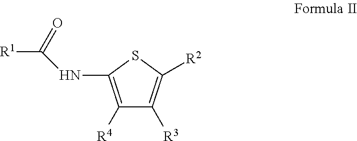

wherein R¹ is aryl, cycloalkenyl, heteroaryl, optionally substituted with a substituent selected from at least one of the following: alkyl, aryl, nitro, —COOH, thioalkyl, thioalkylaryl and halogen;
R² is H or alkyl;
R³ is H, alkyl, or aryl, each of which may be optionally substituted with an alkyl group, and wherein R² and R³ together may be connected to form a cycloalkyl or heterocyclic group, which may be optionally substituted with an alkyl group; and
R₄ is CN, —COO-alkyl, —CO—NH₂, —CO—NH-alkyl, —CO—NH-heterocyclyl, —CO—NH-alkyl-heterocyclyl, or NH₂.

In particular embodiments of active compounds according to Formula II, R₁ is a nitro-substituted furan, and R² and R³ together form a 5-membered cycloalkyl ring. In an embodiment, the furan is attached through the two position and the nitro is at the 5 position of the furanyl ring.

Compounds according to Formula II may be selected to exhibit an inhibitory effect on one or more bacterial DNA helicases, nucleases, or helicase-nuclease enzyme complexes, such as, for example, one or more enzymes selected from the RecBCD and AddAB families of enzymes. Specific examples of compounds according to Formula II include the compounds in Table 4.

TABLE 4
Compounds according to Formula II.

| | |
|---|---|
| 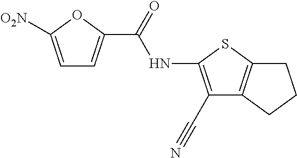 [SMILES: N#Cc1c(NC(=O)c2ccc([N+](=O)[O-])o2)sc2c1CCC2] | Compound 4 |
| 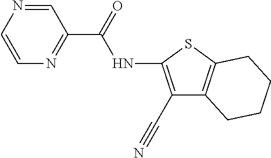 [SMILES: N#Cc1c(NC(=O)c2cnccn2)sc2c1CCCC2] | Compound 5 |
| 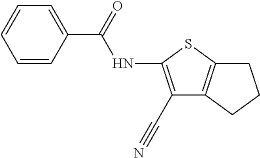 [SMILES: N#Cc1c(NC(=O)c2ccccc2)sc2c1CCC2] | Compound 38 |
| 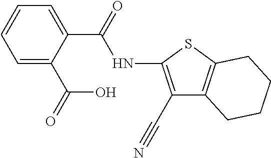 [SMILES: N#Cc1c(NC(=O)c2ccccc2C(=O)O)sc2c1CCCC2] | Compound 39 |
| 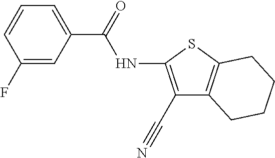 [SMILES: N#Cc1c(NC(=O)c2cccc(F)c2)sc2c1CCCC2] | Compound 40 |
| 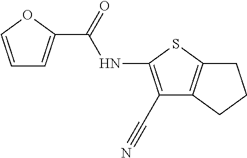 [SMILES: N#Cc1c(NC(=O)c2ccco2)sc2c1CCC2] | Compound 41 |
| 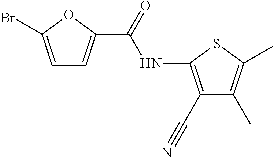 [SMILES: Cc1sc(NC(=O)c2ccc(Br)o2)c(C#N)c1C] | Compound 42 |
| 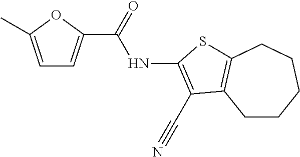 [SMILES: Cc1ccc(C(=O)Nc2sc3c(c2C#N)CCCCC3)o1] | Compound 43 |
| 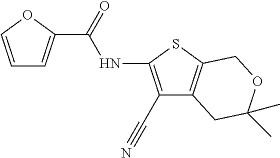 [SMILES: CC1(C)Cc2c(sc(NC(=O)c3ccco3)c2C#N)CO1] | Compound 44 |
| 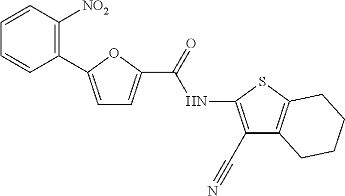 [SMILES: N#Cc1c(NC(=O)c2ccc(-c3ccccc3[N+](=O)[O-])o2)sc2c1CCCC2] | Compound 45 |
| 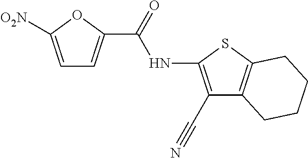 [SMILES: N#Cc1c(NC(=O)c2ccc([N+](=O)[O-])o2)sc2c1CCCC2] | Compound 46 |
| 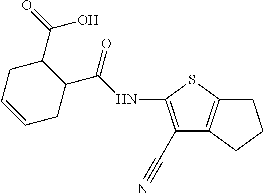 [SMILES: N#Cc1c(NC(=O)C2CC=CCC2C(=O)O)sc2c1CCC2] | Compound 47 |
| 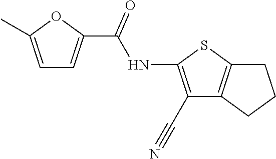 [SMILES: Cc1ccc(C(=O)Nc2sc3c(c2C#N)CCC3)o1] | Compound 48 |
| 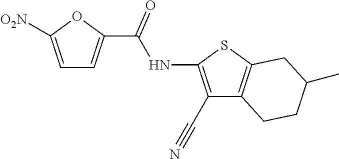 [SMILES: CC1CCc2c(sc(NC(=O)c3ccc([N+](=O)[O-])o3)c2C#N)C1] | Compound 49 |
| 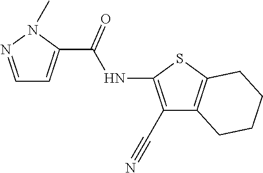 [SMILES: Cn1nccc1C(=O)Nc1sc2c(c1C#N)CCCC2] | Compound 69 |
| 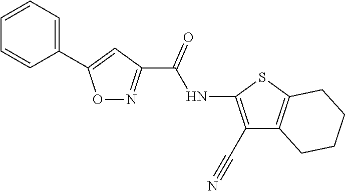 [SMILES: N#Cc1c(NC(=O)c2cc(-c3ccccc3)on2)sc2c1CCCC2] | Compound 70 |
| 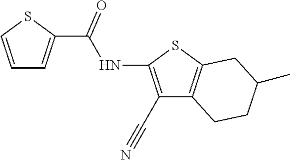 [SMILES: CC1CCc2c(sc(NC(=O)c3cccs3)c2C#N)C1] | Compound 71 |
| 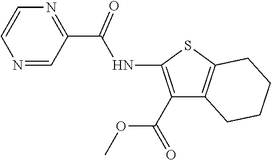 [SMILES: COC(=O)c1c(NC(=O)c2cnccn2)sc2c1CCCC2] | Compound 72 |
| 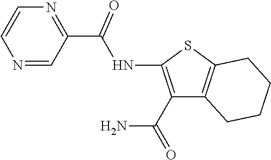 [SMILES: NC(=O)c1c(NC(=O)c2cnccn2)sc2c1CCCC2] | Compound 73 |
| 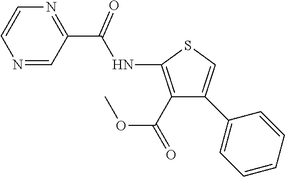 [SMILES: COC(=O)c1c(-c2ccccc2)csc1NC(=O)c1cnccn1] | Compound 74 |
| 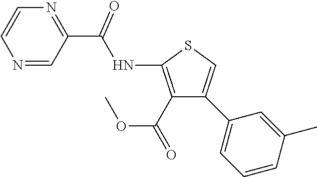 [SMILES: COC(=O)c1c(-c2cccc(C)c2)csc1NC(=O)c1cnccn1] | Compound 75 |
| 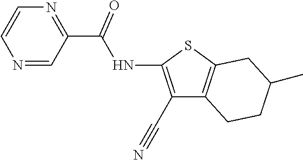 [SMILES: CC1CCc2c(sc(NC(=O)c3cnccn3)c2C#N)C1] | Compound 76 |
| 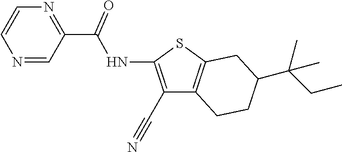 [SMILES: CCC(C)(C)C1CCc2c(sc(NC(=O)c3cnccn3)c2C#N)C1] | Compound 77 |
| 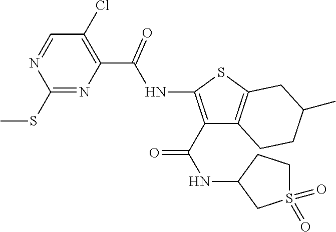 | Compound 78 |
| 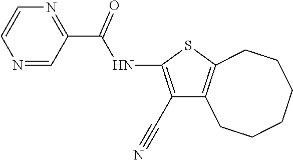 [SMILES: N#Cc1c(NC(=O)c2cnccn2)sc2c1CCCCCC2] | Compound 79 |
| 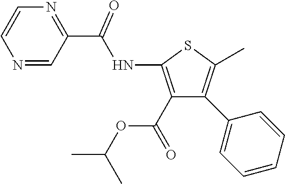 [SMILES: Cc1sc(NC(=O)c2cnccn2)c(C(=O)OC(C)C)c1-c1ccccc1] | Compound 80 |
| 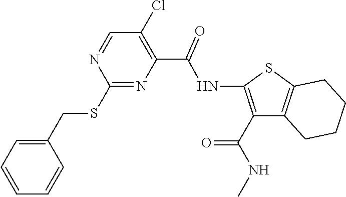 [SMILES: CNC(=O)c1c(NC(=O)c2nc(SCc3ccccc3)ncc2Cl)sc2c1CCCC2] | Compound 81 |
| 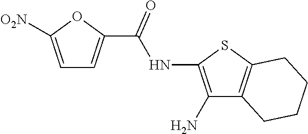 [SMILES: Nc1c(NC(=O)c2ccc([N+](=O)[O-])o2)sc2c1CCCC2] | Compound 82 |
| 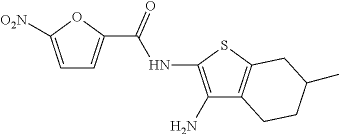 [SMILES: CC1CCc2c(sc(NC(=O)c3ccc([N+](=O)[O-])o3)c2N)C1] | Compound 83 |
| 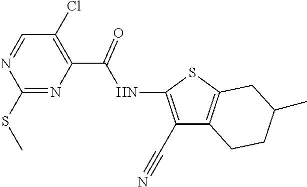 [SMILES: CSc1ncc(Cl)c(C(=O)Nc2sc3c(c2C#N)CCC(C)C3)n1] | Compound 84 |
| 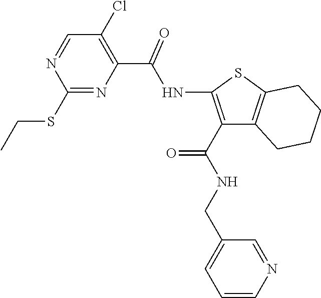 [SMILES: CCSc1ncc(Cl)c(C(=O)Nc2sc3c(c2C(=O)NCc2cccnc2)CCCC3)n1] | Compound 85 |

In further embodiments, active compounds that inhibit bacterial helicase(s) are selected from a compound of structural class C (the “nitrofurans”), according to Formula III:

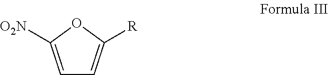

wherein R is selected from at least one of the following: —CO—O-alkyl heteroaryl, —CO—NH-heteroaryl, alkenyl heteroaryl, —CO—O-alkyl-CO—NH-heteroaryl, —CO—NH-aryl, and —CO—NH-alkyl aryl, any of which may be optionally substituted with a substituent selected from at least one of the following: C═O, N—CO-alkyl, CN, alkyl, —CONH₂, heterocyclyl or —NH—CO-haloaryl.

Compounds according to Formula III may be selected to exhibit an inhibitory effect on one or more bacterial DNA helicases, nucleases, or helicase-nuclease enzyme complexes, such as, for example, one or more enzymes selected from the RecBCD and AddAB families of enzymes. Specific examples of compounds according to Formula III include the compounds in Table 5.

TABLE 5
Compounds according to Formula III.

| | |
|---|---|
| 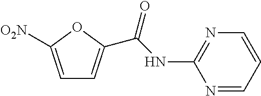 [SMILES: O=C(Nc1ncccn1)c1ccc([N+](=O)[O-])o1] | Compound 6 |
| 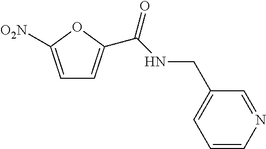 [SMILES: O=C(NCc1cccnc1)c1ccc([N+](=O)[O-])o1] | Compound 7 |
| 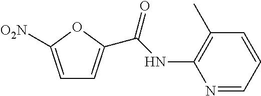 [SMILES: Cc1cccnc1NC(=O)c1ccc([N+](=O)[O-])o1] | Compound 8 |
| 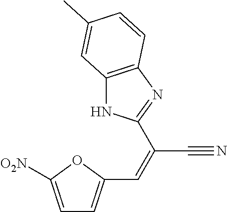 [SMILES: Cc1ccc2nc(/C(C#N)=C\c3ccc([N+](=O)[O-])o3)[nH]c2c1] | Compound 9 |
| 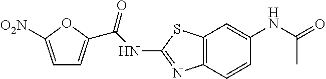 [SMILES: CC(=O)Nc1ccc2nc(NC(=O)c3ccc([N+](=O)[O-])o3)sc2c1] | Compound 10 |
| 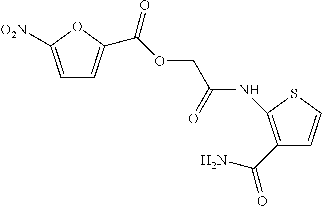 [SMILES: NC(=O)c1ccsc1NC(=O)COC(=O)c1ccc([N+](=O)[O-])o1] | Compound 11 |
| 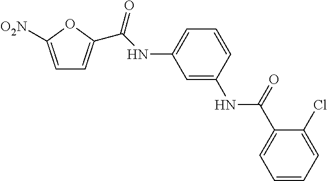 [SMILES: O=C(Nc1cccc(NC(=O)c2ccccc2Cl)c1)c1ccc([N+](=O)[O-])o1] | Compound 12 |
| 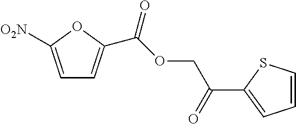 [SMILES: O=C(OCC(=O)c1cccs1)c1ccc([N+](=O)[O-])o1] | Compound 13 |
| 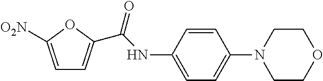 [SMILES: O=C(Nc1ccc(N2CCOCC2)cc1)c1ccc([N+](=O)[O-])o1] | Compound 14 |

In further embodiments, active compounds that inhibit bacterial helicase(s) are selected from a compound of structural class D (the “nitrothiazoles”), according to Formula IV:

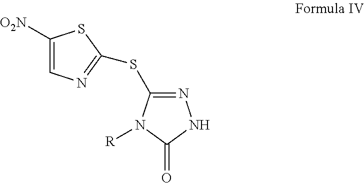

wherein R is an alkyl or alkenyl group.

Compounds according to Formula IV may be selected to exhibit an inhibitory effect on one or more bacterial DNA helicases, nucleases, or helicase-nuclease enzyme complexes, such as, for example, one or more enzymes selected from the RecBCD and AddAB families of enzymes. An example of a compound according to Formula IV is compound 15 as shown in Table 6.

TABLE 6
Compound according to Formula IV.

| | |
|---|---|
| 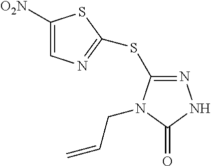 [SMILES: C=CCn1c(Sc2ncc([N+](=O)[O-])s2)n[nH]c1=O] | Compound 15 |

In further embodiments, active compounds that inhibit bacterial helicase(s) are selected from a compound of structural class E (the “iminobenzothiazoles”), according to Formula V:

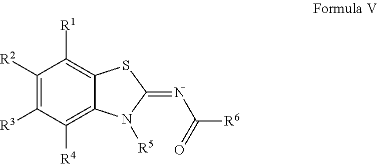

wherein R¹ is H;
R² is H, halo, alkyl, CONH-alkyl, nitro, CO₂-alkyl, SO₂-alkyl or SO₂NH₂,
R³ is H;
R⁴ is H, halo, alkyl, or alkoxy;
R⁵ is alkyl, alkenyl, alkynyl, alkyl alkoxy, or alkyl-CO-alkoxy; and
R⁶ is aryl, alkyl aryl, alkenyl aryl, alkenyl heteroaryl, alkyl-SO₂-aryl, alkyl-O-aryl, aryl-SO₂-heterocyclyl, heteroaryl, heterocyclyl, cycloalkyl, diphenyl or heterocycloalkenyl, any of which may be optionally substituted with a substituent selected from at least one of the following: nitro, halo, alkyl, alkoxy, aryl, —CO, —CO₂-alkyl, CO-substituted heterocyclyl, methylenedioxy, SO₂-alkyl, or halophenyl-substituted heteroaryl.

Compounds according to Formula V may be selected to exhibit an inhibitory effect on one or more bacterial DNA helicases, nucleases, or helicase-nuclease enzyme complexes, such as, for example, one or more enzymes selected from the RecBCD and AddAB families of enzymes. Examples of compounds according to Formula V include compounds shown in Table 7.

TABLE 7
Compounds according to Formula V.

| | |
|---|---|
| 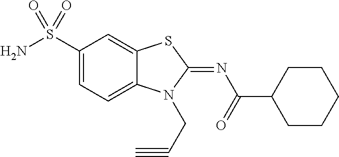 [SMILES: C#CCn1/c(=N\C(=O)C2CCCCC2)sc2cc(S(N)(=O)=O)ccc21] | Compound 16 |
| 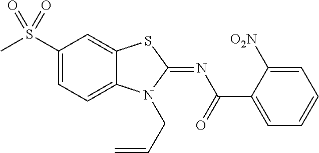 [SMILES: C=CCn1/c(=N\C(=O)c2ccccc2[N+](=O)[O-])sc2cc(S(C)(=O)=O)ccc21] | Compound 17 |
| 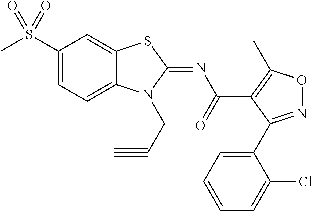 [SMILES: C#CCn1/c(=N\C(=O)c2c(-c3ccccc3Cl)noc2C)sc2cc(S(C)(=O)=O)ccc21] | Compound 18 |
| 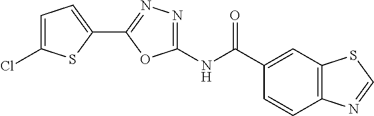 [SMILES: O=C(Nc1nnc(-c2ccc(Cl)s2)o1)c1ccc2ncsc2c1] | Compound 86 |
| 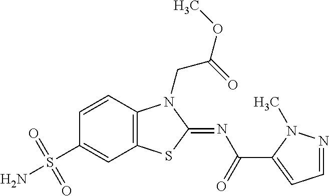 [SMILES: COC(=O)Cn1/c(=N/C(=O)c2ccnn2C)sc2cc(S(N)(=O)=O)ccc21] | Compound 87 |
| 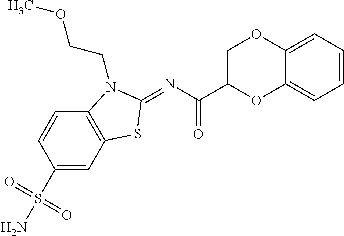 [SMILES: COCCn1/c(=N/C(=O)C2COc3ccccc3O2)sc2cc(S(N)(=O)=O)ccc21] | Compound 88 |
| 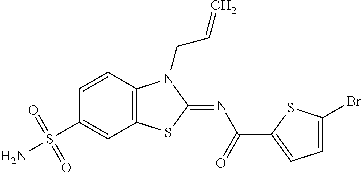 [SMILES: C=CCn1/c(=N/C(=O)c2ccc(Br)s2)sc2cc(S(N)(=O)=O)ccc21] | Compound 89 |
| 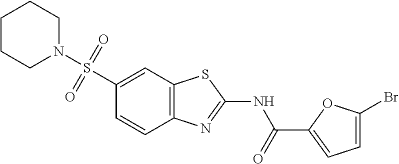 [SMILES: O=C(Nc1nc2ccc(S(=O)(=O)N3CCCCC3)cc2s1)c1ccc(Br)o1] | Compound 90 |
| 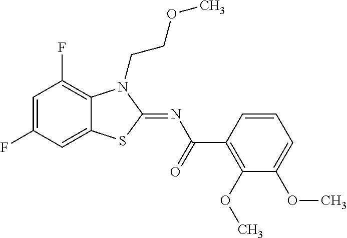 [SMILES: COCCn1/c(=N/C(=O)c2cccc(OC)c2OC)sc2cc(F)cc(F)c21] | Compound 91 |
| 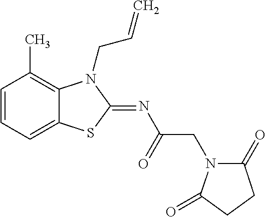 [SMILES: C=CCn1/c(=N/C(=O)CN2C(=O)CCC2=O)sc2cccc(C)c21] | Compound 92 |
| 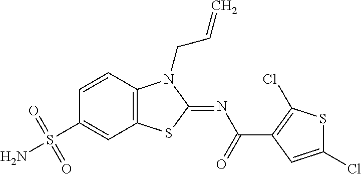 [SMILES: C=CCn1/c(=N/C(=O)c2cc(Cl)sc2Cl)sc2cc(S(N)(=O)=O)ccc21] | Compound 93 |
| 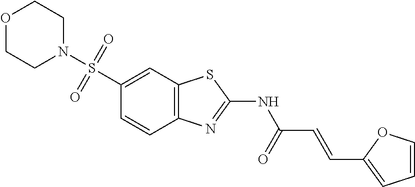 [SMILES: O=C(/C=C/c1ccco1)Nc1nc2ccc(S(=O)(=O)N3CCOCC3)cc2s1] | Compound 94 |
| 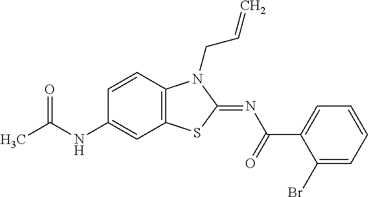 [SMILES: C=CCn1/c(=N/C(=O)c2ccccc2Br)sc2cc(NC(C)=O)ccc21] | Compound 95 |
| 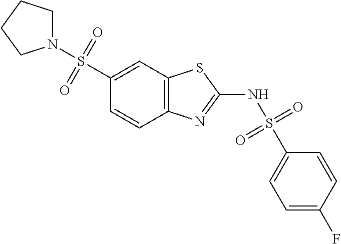 [SMILES: O=S(=O)(Nc1nc2ccc(S(=O)(=O)N3CCCC3)cc2s1)c1ccc(F)cc1] | Compound 96 |
| 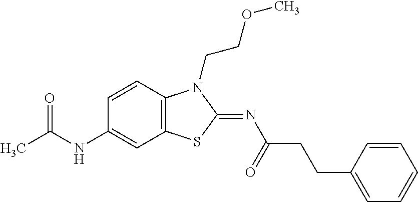 [SMILES: COCCn1/c(=N/C(=O)CCc2ccccc2)sc2cc(NC(C)=O)ccc21] | Compound 97 |
| 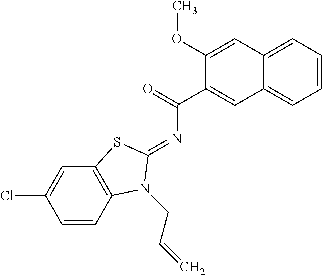 [SMILES: C=CCn1/c(=N/C(=O)c2cc3ccccc3cc2OC)sc2cc(Cl)ccc21] | Compound 98 |
| 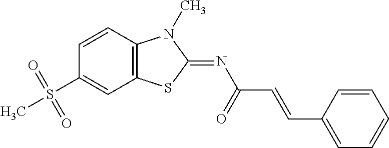 [SMILES: Cn1/c(=N/C(=O)/C=C/c2ccccc2)sc2cc(S(C)(=O)=O)ccc21] | Compound 99 |
| 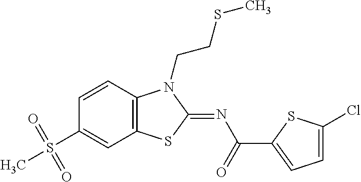 [SMILES: CSCCn1/c(=N/C(=O)c2ccc(Cl)s2)sc2cc(S(C)(=O)=O)ccc21] | Compound 100 |
| 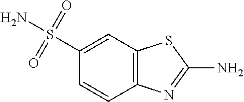 [SMILES: Nc1nc2ccc(S(N)(=O)=O)cc2s1] | Compound 101 |
| 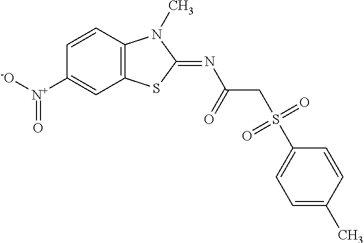 | Compound 102 |
| 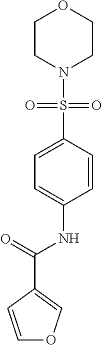 [SMILES: O=C(Nc1ccc(S(=O)(=O)N2CCOCC2)cc1)c1ccoc1] | Compound 103 |
| 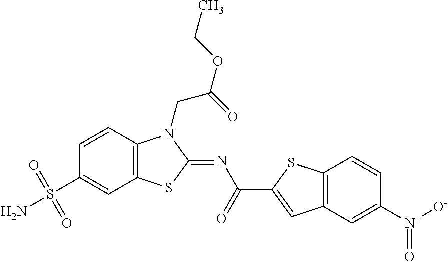 [SMILES: CCOC(=O)Cn1/c(=N/C(=O)c2cc3cc([N+](=O)[O-])ccc3s2)sc2cc(S(N)(=O)=O)ccc21] | Compound 104 |
| 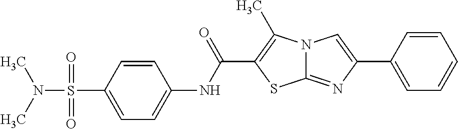 [SMILES: Cc1c(C(=O)Nc2ccc(S(=O)(=O)N(C)C)cc2)sc2nc(-c3ccccc3)cn12] | Compound 105 |
| 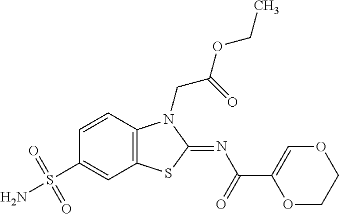 [SMILES: CCOC(=O)Cn1/c(=N/C(=O)C2=COCCO2)sc2cc(S(N)(=O)=O)ccc21] | Compound 106 |
| 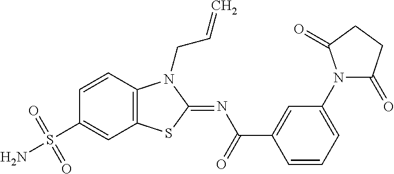 [SMILES: C=CCn1/c(=N/C(=O)c2cccc(N3C(=O)CCC3=O)c2)sc2cc(S(N)(=O)=O)ccc21] | Compound 107 |
| 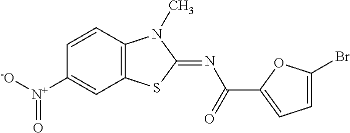 | Compound 108 |
| 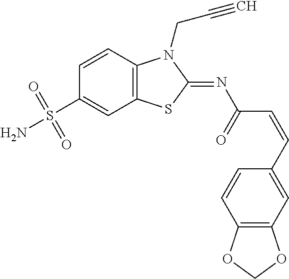 [SMILES: C#CCn1/c(=N/C(=O)/C=C\c2ccc3c(c2)OCO3)sc2cc(S(N)(=O)=O)ccc21] | Compound 109 |
| 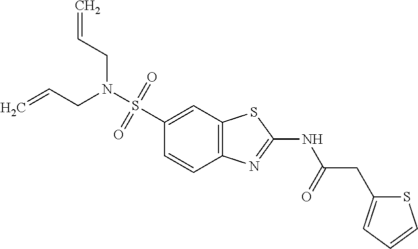 [SMILES: C=CCN(CC=C)S(=O)(=O)c1ccc2nc(NC(=O)Cc3cccs3)sc2c1] | Compound 110 |
| 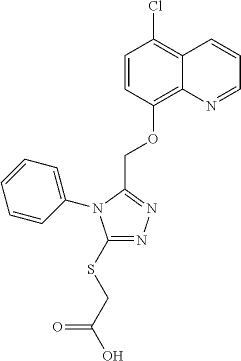 [SMILES: O=C(O)CSc1nnc(COc2ccc(Cl)c3cccnc23)n1-c1ccccc1] | Compound 111 |
| 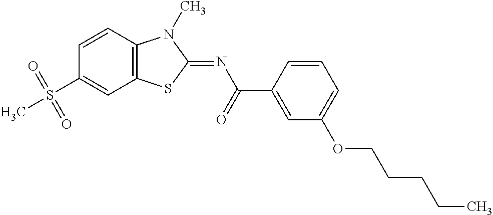 [SMILES: CCCCCOc1cccc(C(=O)/N=c2\sc3cc(S(C)(=O)=O)ccc3n2C)c1] | Compound 112 |
| 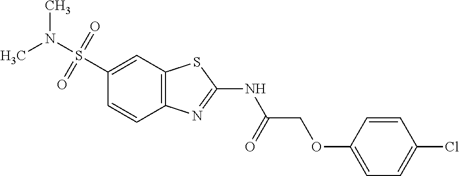 [SMILES: CN(C)S(=O)(=O)c1ccc2nc(NC(=O)COc3ccc(Cl)cc3)sc2c1] | Compound 113 |
| 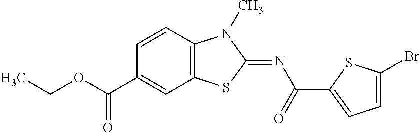 [SMILES: CCOC(=O)c1ccc2c(c1)s/c(=N\C(=O)c1ccc(Br)s1)n2C] | Compound 114 |
| 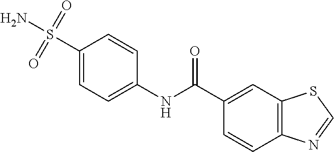 [SMILES: NS(=O)(=O)c1ccc(NC(=O)c2ccc3ncsc3c2)cc1] | Compound 115 |
| 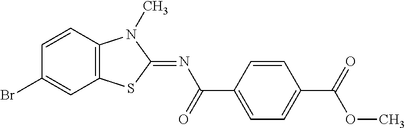 [SMILES: COC(=O)c1ccc(C(=O)/N=c2\sc3cc(Br)ccc3n2C)cc1] | Compound 116 |
| 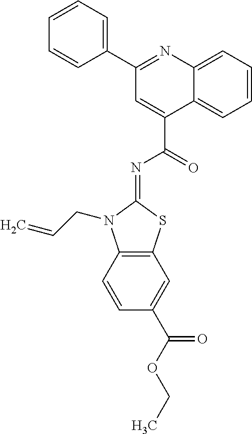 [SMILES: C=CCn1/c(=N/C(=O)c2cc(-c3ccccc3)nc3ccccc23)sc2cc(C(=O)OCC)ccc21] | Compound 117 |
| 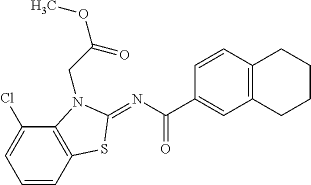 [SMILES: COC(=O)Cn1/c(=N/C(=O)c2ccc3c(c2)CCCC3)sc2cccc(Cl)c21] | Compound 118 |
| 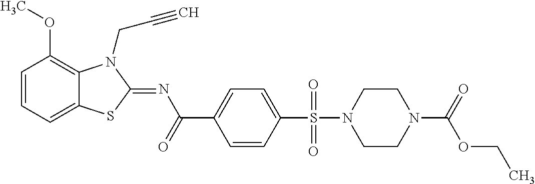 [SMILES: C#CCn1/c(=N/C(=O)c2ccc(S(=O)(=O)N3CCN(C(=O)OCC)CC3)cc2)sc2cccc(OC)c21] | Compound 119 |
| 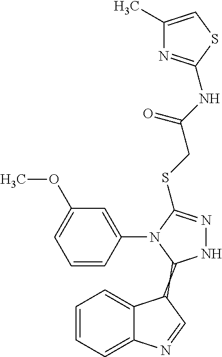 [SMILES: COc1cccc(N2C(SCC(=O)Nc3nc(C)cs3)=NNC2=C2C=Nc3ccccc32)c1] | Compound 120 |
| 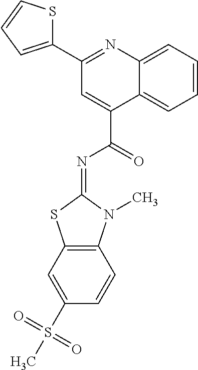 [SMILES: Cn1/c(=N\C(=O)c2cc(-c3cccs3)nc3ccccc23)sc2cc(S(C)(=O)=O)ccc21] | Compound 121 |
| 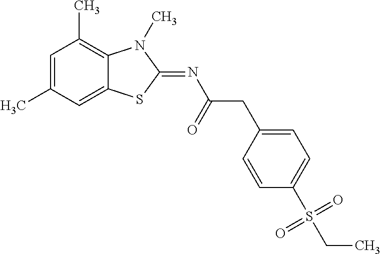 [SMILES: CCS(=O)(=O)c1ccc(CC(=O)/N=c2\sc3cc(C)cc(C)c3n2C)cc1] | Compound 122 |
| 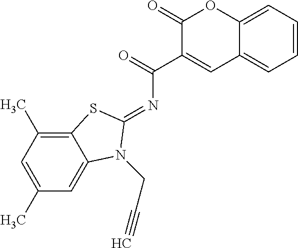 [SMILES: C#CCn1/c(=N/C(=O)c2cc3ccccc3oc2=O)sc2c(C)cc(C)cc21] | Compound 123 |
| 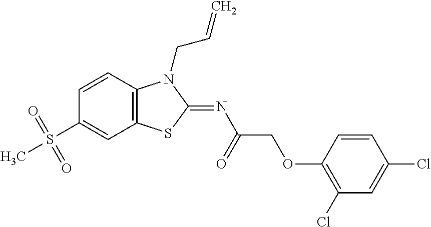 [SMILES: C=CCn1/c(=N/C(=O)COc2ccc(Cl)cc2Cl)sc2cc(S(C)(=O)=O)ccc21] | Compound 124 |
| 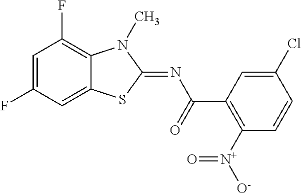 [SMILES: Cn1/c(=N/C(=O)c2cc(Cl)ccc2[N+](=O)[O-])sc2cc(F)cc(F)c21] | Compound 125 |
| 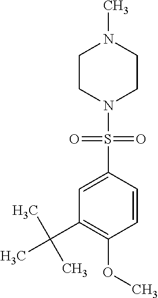 [SMILES: COc1ccc(S(=O)(=O)N2CCN(C)CC2)cc1C(C)(C)C] | Compound 126 |
| 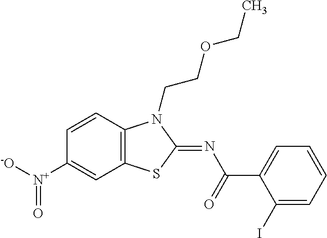 | Compound 127 |
| 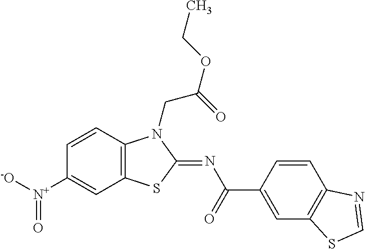 | Compound 128 |
| 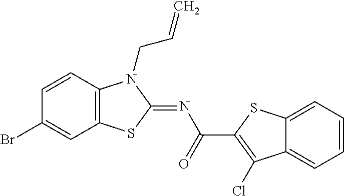 [SMILES: C=CCn1/c(=N/C(=O)c2sc3ccccc3c2Cl)sc2cc(Br)ccc21] | Compound 129 |
| 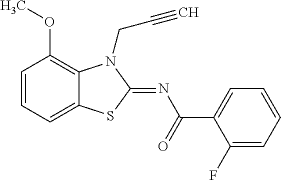 [SMILES: C#CCn1/c(=N/C(=O)c2ccccc2F)sc2cccc(OC)c21] | Compound 130 |
| 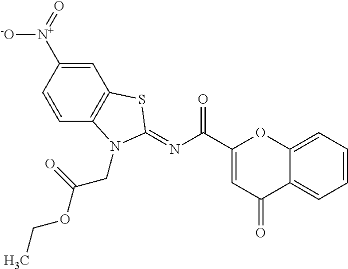 | Compound 131 |
| 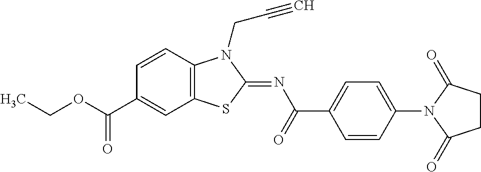 [SMILES: C#CCn1/c(=N/C(=O)c2ccc(N3C(=O)CCC3=O)cc2)sc2cc(C(=O)OCC)ccc21] | Compound 132 |
| 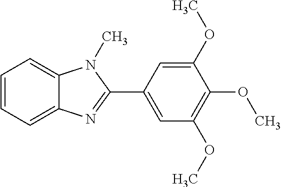 [SMILES: COc1cc(-c2nc3ccccc3n2C)cc(OC)c1OC] | Compound 133 |
| 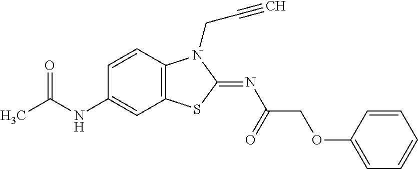 [SMILES: C#CCn1/c(=N/C(=O)COc2ccccc2)sc2cc(NC(C)=O)ccc21] | Compound 134 |
| 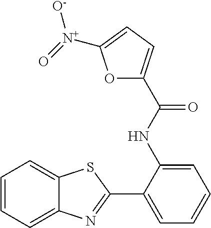 [SMILES: O=C(Nc1ccccc1-c1nc2ccccc2s1)c1ccc([N+](=O)[O-])o1] | Compound 135 |
| 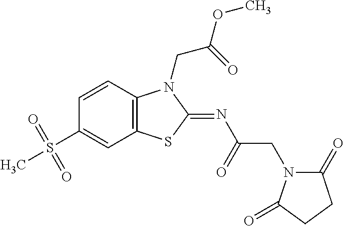 [SMILES: COC(=O)Cn1/c(=N/C(=O)CN2C(=O)CCC2=O)sc2cc(S(C)(=O)=O)ccc21] | Compound 136 |
| 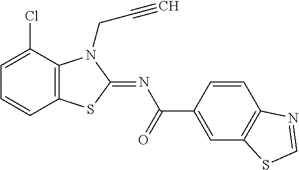 [SMILES: C#CCn1/c(=N/C(=O)c2ccc3ncsc3c2)sc2cccc(Cl)c21] | Compound 137 |
| 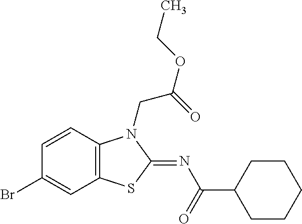 [SMILES: CCOC(=O)Cn1/c(=N/C(=O)C2CCCCC2)sc2cc(Br)ccc21] | Compound 138 |
| 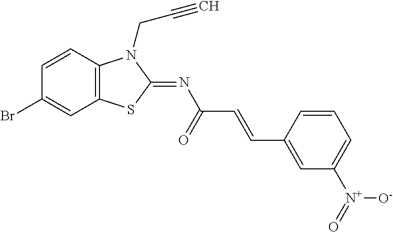 [SMILES: C#CCn1/c(=N/C(=O)/C=C/c2cccc([N+](=O)[O-])c2)sc2cc(Br)ccc21] | Compound 139 |
| 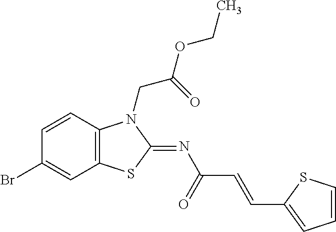 [SMILES: CCOC(=O)Cn1/c(=N/C(=O)/C=C/c2cccs2)sc2cc(Br)ccc21] | Compound 140 |
| 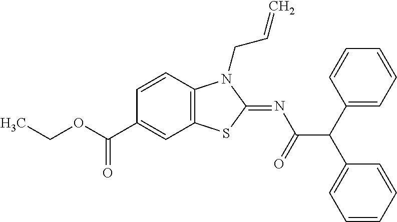 [SMILES: C=CCn1/c(=N/C(=O)C(c2ccccc2)c2ccccc2)sc2cc(C(=O)OCC)ccc21] | Compound 141 |
| 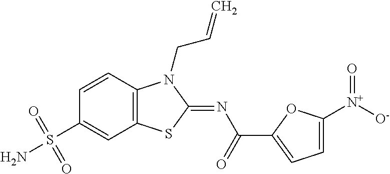 [SMILES: C=CCn1/c(=N/C(=O)c2ccc([N+](=O)[O-])o2)sc2cc(S(N)(=O)=O)ccc21] | Compound 142 |

It is to be understood that any pharmaceutically acceptable salts, esters, isomers or solvates of active compounds as described herein are contemplated and included within the scope of the present disclosure.

Exemplary compounds of the present invention may possess chiral or asymmetric carbon atoms (optical centers) or double bonds; the racemates, diastereomers, geometric isomers and individual optical isomers are all intended to be encompassed within the scope of this disclosure. In embodiments, the compounds described herein can also include all isotopes of atoms occurring in the final compounds. Isotopes include those atoms having the same atomic number but different mass numbers. For example, isotopes of hydrogen include tritium and deuterium. Further, the compounds described herein may include tautomeric forms, such as keto-enol tautomers. Tautomeric forms can be in equilibrium or sterically locked into one form by appropriate substitution. For example, the tautomers of compounds of structure A, the pyrimidopyridones, are included.

In certain embodiments, the active compounds described herein may have an IC₅₀ for a bacterial helicase selected from less than about 100 micromol/liter (100 μM), less than about 50 micromol/liter (50 μM), less than about 10 micromol/liter (10 μM), and less than about 1 micromol/liter (1 μM). In such embodiments, the bacterial helicase may be selected from one or both of a RecBCD and an AddAB helicase. In other embodiments, the active compounds described herein may be a dual function compound that exhibits an IC₅₀ for a bacterial helicase selected from less than about 100 micromol/liter (100 μM), less than about 50 micromol/liter (50 μM), less than about 10 micromol/liter (10 μM), and less than about 1 micromol/liter (1 μM), and an IC₅₀ for bacterial gyrase selected from less than about 100 micromol/liter (100 μM), less than about 50 micromol/liter (50 μM), less than about 10 micromol/liter (10 μM), and less than about 1 micromol/liter (1 μM). Again, in such embodiments, the bacterial helicase may be selected from one or both of a RecBCD and a AddAB helicase.

In further embodiments, an active compound as described herein may be characterized as an agent or composition that causes a measurable change in bacterial growth, viability, or survival. For example, active compounds as described herein may be characterized as a compound that causes a greater than 2-fold change, greater than 5-fold change, greater than 10-fold change, greater than 15-fold change, and greater than 20-fold change in bacterial growth, viability or survival.

Without being bound by a particular theory, inhibitors of AddAB and RecBCD may be useful antibacterial drugs for at least two reasons. First, these enzymes are required for repair of DNA damage inflicted upon bacteria by their host cells upon infection. Salmonella recB mutants are much less able than wild type to kill a mouse (Buchmeier, N. A.; Lipps, C. J.; So, M. Y.; Heffron, F., Mol Microbiol 1993, 7 (6), 933-6; Cirz, R. T.; Chin, J. K.; Andes, D. R.; de Crecy-Lagard, V.; Craig, W. A.; Romesberg, F. E., PLoS Biol 2005, 3 (6), e176), and H. pylori addAB mutants less effectively colonize the mouse stomach than wild type (Amundsen, S. K.; Fero, J.; Hansen, L. M.; Cromie, G. A.; Solnick, J. V.; Smith, G. R.; Salama, N. R., Molec. Microb. 2008, 69, 994-1007). Second, RecBCD, and perhaps AddAB, is required for the induction of the SOS response to DNA damage, which includes the induction of mutagenic polymerases responsible for most induced mutations (McPartland, A.; Green, L.; Echols, H., Control of recA gene RNA in E. coli: regulatory and signal genes. Cell 1980, 20, 731-737). Inhibition of RecBCD and AddAB should thus lessen the evolution of bacteria resistant to the inhibitor, an important goal in current antibacterial drug therapy.

The AddAB and RecBCD class of enzymes is found in about 90% of all bacterial species whose genomes have been sequenced and reported (Cromie, G. A., J Bacteriol 2009, 191 (16), 5076-84). Compound 1 and its derivatives may be especially effective, since, for example, they contain a pyrimidopyridone moiety that has been found to inhibit DNA gyrase and creates dsDNA breaks whose repair requires the RecBCD enzyme (Cirz, R. T.; Chin, J. K.; Andes, D. R.; de Crecy-Lagard, V.; Craig, W. A.; Romesberg, F. E., PLoS Biol 2005, 3 (6), e176). Therefore, administration of such compounds can lead to both DNA damage and the failure to repair it. In such embodiments, the compounds described herein provide a single-molecule capable of providing a combination of antibacterial functions.

### III. Compositions

Pharmaceutical compositions are provided herein. Pharmaceutical compositions according to the present description include a pharmaceutically acceptable carrier and a therapeutically effective amount of an active compound according to the present description. The pharmaceutical compositions can take the form of, for example, solutions, suspensions, emulsion, tablets, pills, capsules, powders, sustained-release formulations, or suppositories. Examples of suitable pharmaceutical carriers are described in, for example, Remington's Pharmaceutical Sciences, by E. W. Martin. The pharmaceutical compositions disclosed herein may be prepared for administration by any suitable route known to the skilled artisan including, for example, intravenous, subcutaneous, intramuscular, intradermal, transdermal, intrathecal, intracerebral, intraperitoneal, intranasal, epidural, pulmonary, and oral routes. Administration can be immediate or rapid, such as by injection, or carried out over a period of time, such as by infusion or administration of controlled or delayed release formulations.

Where pharmaceutical formulations are prepared for treating tissues in the central nervous system, administration can be by injection or infusion into the cerebrospinal fluid (CSF). Moreover, where pharmaceutical compositions are prepared for delivery to cells or tissues in the central nervous system, the pharmaceutical composition may be formulated to include one or more other carriers or components capable of promoting penetration of the active compound or a derivative of the active compound across the blood-brain barrier.

When prepared for oral administration, the pharmaceutical compositions described herein may be prepared, for example, in capsules, tablets, caplets, lozenges, and aqueous suspensions or solutions. Pharmaceutical compositions described herein prepared for oral administration can be formulated using known carriers, including known fillers, diluents, excipients, binders, surfactants, suspending agents, emulsifiers, lubricants, sweeteners, flavorants, and colorants, suited to formulation of the desired dosage form. Additionally, pharmaceutical compositions as described herein can be prepared using formulation approaches that utilize encapsulation in liposomes, microparticles, microcapsules, receptor-mediated endocytosis (see, e.g., Wu et al. J. Biol. Chem. 262:4429-32, 1987), to facilitate delivery or uptake of the active compound.

Examples of pharmaceutically acceptable carriers include sterile liquids, such as water and oils, including those of petroleum, animal, vegetable or synthetic origin, such as peanut oil, soybean oil, mineral oil, and sesame oil. Aqueous carriers, including water, are typical carriers for pharmaceutical compositions prepared for intravenous administration. As further examples, saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, and ethanol. The composition, if desired, can also contain wetting or emulsifying agents, or pH buffering agents.

The pharmaceutical compositions described herein can be formulated using any of the active compounds described herein, including any pharmaceutically acceptable salts, esters, isomers or solvates thereof. In certain embodiments, the pharmaceutical compositions described herein include an active compound as described herein, and in alternative embodiments, the pharmaceutical compositions include two or more active compounds according to the present description. The amount of the one or more active compounds included in the pharmaceutical composition will vary, depending upon, for example, the nature and activity of the active compound(s), the nature and composition of the dosage form, and the desired dose to be administered to a subject.

In some instances, it can be desirable to administer the compositions described herein locally to the area in need of treatment. Local administration can be achieved by, for example, and not by way of limitation, local infusion during surgery, topical application (e.g., in conjunction with a wound dressing after surgery), by injection, by means of a catheter, by means of a suppository, or by means of an implant, the implant being of a porous, non-porous, or gelatinous material, including membranes such as silastic membranes, or fibers. In one embodiment, administration can be by direct injection at the site of bacterial infection.

In another embodiment, the agent can be delivered in a vesicle, in particular a liposome (see, e.g., Langer, Science 249:1527 33 (1990); Treat et al., In Liposomes in the Therapy of Infectious Disease and Cancer, Lopez-Berestein and Fidler (eds.), Liss, New York, pp. 353 65 (1989); Lopez-Berestein, supra, pp. 317 27).

In yet another embodiment, the agent can be delivered in a controlled release system. In one embodiment, a pump can be used (see, e.g., Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, thus requiring only a fraction of the systemic dose (see, e.g., Goodson, Medical Applications of Controlled Release, supra, Vol. 2, pp. 115 138 (1984)). Other controlled release systems are discussed in, for example, Langer (Science 249:1527 33 (1990)).

In addition to one or more active compounds as described herein and a pharmaceutical carrier, pharmaceutical compositions according to the present description may include one or more additional therapeutic or prophylactic agents. Examples of such agents include amifloxacin, cinoxacin, ciprofloxacin, danofloxacin, difloxacin, enoxacin, enrofloxacin, fleroxacin, irloxacin, lomefloxacin, miloxacin, norfloxacin, ofloxacin, pefloxacin, rosoxacin, rufloxacin, sarafloxacin, gatifloxacin, sparfloxacin, temafloxacin, tosufloxacin, tobramycin, colistin, azithromycin, amikacin, cefaclor (Ceclor), aztreonam, amoxicillin, ceftazidime, cephalexin (Keflex), gentamicin, vancomycin, imipenem, doripenem, piperacillin, minocycline, erythromycin, an aminoglycoside, a tetracycline, a sulfonamide, p-aminobenzoic acid, a diaminopyrimidine, a quinolone, a.beta-lactam, a.beta-lactam and/or a.beta-lactamase inhibitor, chloraphenicol, a macrolide, penicillins, cephalosporins, corticosteroid, prostaglandin, linomycin, clindamycin, spectinomycin, polymyxin B, colistin, bacitracin, isoniazid, rifampin, ethambutol, ethionamide, aminosalicylic acid, cycloserine, capreomycin, a sulfone, clofazimine, thalidomide, a polyene antifungal, flucytosine, imidazole, triazole, griseofulvin, terconazole, butoconazole ciclopirax, ciclopirox olamine, haloprogin, tolnaftate, naftifine, or terbinafine, either individually or in any combination.

It should be understood, however, that a specific dosage and treatment regime for any particular subject or disease state will depend upon a variety of factors, including the age, body weight, general health, sex, diet, time of administration, nature of active compound(s), rate of excretion, drug combination, the judgment of the treating physician, and the severity of the particular disease and/or microorganism being treated. Moreover, determination of the amount of a pharmaceutical composition to be administered to a subject will depend upon, among other factors, the amount and specific activity of the active compound(s) included in the pharmaceutical composition and the use or incorporation of additional therapeutic or prophylactic agents or treatment regimes. Determination of therapeutically effective dosages may be based on animal model studies and is typically guided by determining effective dosages and administration protocols that significantly reduce the occurrence or severity of bacterial growth in model subjects. A non-limiting range for a therapeutically effective amount of the active compounds described herein is from about 0.001 mg/kg to about 100 mg/kg body weight per day. For example, pharmaceutical compositions according to the present description can be prepared and administered such that the amount of active compound according to the present description administered to a subject is selected from between about 0.001 mg/kg and about 50 mg/kg, between about 0.01 mg/kg and about 20 mg/kg, between about 0.1 and about 10 mg/kg, and between about 0.1 mg/kg and about 5 mg/kg body weight per day.

### IV. Methods of Identifying Active Compounds

Methods are also provided to identify agents that selectively inhibit bacterial RecBCD and/or AddAB activity. In one embodiment, the method generally comprises the steps of analyzing a candidate compound in a series of enzymatic and bacterial growth assays, and determining whether the candidate compound selectively inhibits the growth of bacteria.

In one embodiment, an assay disclosed herein is based on the ability of phage T4 gene 2 mutants to grow in E. coli only if the RecBCD nuclease is inactivated by mutation (Oliver, D. B.; Goldberg, E. B., J. Mol. Biol. 1977, 116, 877-881) (FIG. 1). The nuclease, which resides in the RecB polypeptide (Yu, M.; Souaya, J.; Julin, D. A., J. Mol. Biol. 1998, 283, 797-808), is active only if the RecD subunit is present and only if the RecB helicase is active (Hsieh, S.; Julin, D. A., Nucleic Acids Res. 1992, 20 (21), 5647-5653; Amundsen, S. K.; Taylor, A. F.; Chaudhury, A. M.; Smith, G. R., Proc. Natl. Acad. Sci. USA 1986, 83, 5558-5562). The RecB helicase in turn is highly active only in the presence of the RecC subunit (Masterson, C.; Boehmer, P. E.; McDonald, F.; Chaudhuri, S.; Hickson, I. D.; Emmerson, P. T., J. Biol. Chem. 1992, 267 (19), 13564-13572).

In particular embodiments, the primary screen is an enzymatic assay to test candidate compounds for activity against AddAB activity in, for example, V3065 (addAB⁺) and V3069 (vector control) strains of E. coli, in the presence of a T4 gene 2 am149 triple nonsense mutant phage. If a compound is confirmed to be active in the primary screen via retesting, the compound is further tested in a RecBCD enzymatic counterscreening assay and/or a bacterial viability counterscreening assay. The RecBCD enzymatic counterscreen is similar to the primary screen, but uses, for example, V66 (recBCD⁺) and V67 (recB21) strains of E. coli instead of the V3065/V3069 strains. It should be understood that, although the methods and screens provided herein are described in the context of certain materials, such as bacterial strains, the screens and methods are not limited to the specific materials and organisms described. For example, the methods and screens may utilize other bacterial strains to carry out the methods and obtain the desired information.

The bacterial viability counterscreen is similar to the primary screen, but without the presence of the T4 phage. Compounds which show adequate activity in these screens are further analyzed by running additional screens with purified enzymes (a titration assay with AddAB and a selectivity assay with RecBCD, for example), and a general cytotoxicity assay against the V3065 strain. In addition, dual function compounds as described herein may be screened by further assessing compounds that exhibit RecBCD and/or AddAB inhibition for activity against bacterial DNA gyrase.

A skilled artisan would understand that any strain of E. coli expressing recBCD or addAB genes from any species may be used in the primary screen. Similarly, any appropriate phage in any bacterial host may be used, and any phage derived from T4 or a related phage may be used.

In an embodiment, a compound may be deemed as active in the above-described screens, if it shows an inhibition greater than the average percent inhibition of the set of compounds screened. In other embodiments, a compound may deemed active if it exhibits an inhibition greater than the average percent inhibition of the set of compounds screened plus a significant increase in the standard deviation in the primary and confirmatory screens. For purposes of the present disclosure a “significant increase” in the standard deviation may be selected from at least 1.5 times, at least 2 times, at least 2.5 times, and at least 3 times the standard deviation. In the counterscreen using RecBCD, a compound may be deemed active if it shows an inhibition greater than the average percent inhibition of all DMSO-only wells tested. In other embodiments, a compound may be deemed active if it exhibits an inhibition greater than the average percent inhibition of all DMSO-only wells plus a significant increase in the standard deviation. In particular embodiments of a counterscreen using E. coli, a compound may be deemed active if it shows an inhibition greater than the average percent inhibition of all DMSO-only wells tested plus a significant increase in the standard deviation. In specific embodiments, in the titration screen, a compound may be deemed active if it shows an IC₅₀<10 μM. In further embodiments, in the RedBCD selectivity screen and the E. coli cytotoxicity screen, a compound may be deemed active if it shows an IC₅₀>10 μM. In particular embodiments, the threshold values for deeming a compound to be active may be different than those listed above.

A compound may be further analyzed in genetic recombination assays, including an E. coli Hfr recombination assay, a phage λ recombination assay, and/or a Chi hotspot activity to help determine the specific type of inhibition exhibited (e.g., to differentiate the helicase vs. nuclease activity of the AddAB or RecBCD enzyme). The assays described herein may also be useful for applications outside of bacteria, for example, to assay in any way for inhibition of RecBCD and/or AddAB enzyme activity in cells. In an embodiment, the inhibition of RecBCD and/or AddAB may occur directly in a purified enzyme assay and intermediate assays may be skipped.

For purposes of providing a specific example of a screening process carried out according to an embodiment of the methods described herein, a detailed description of such a process is provided. In particular, a high-throughput screen based on the ability of phage T4 gene 2 mutants to grow in Escherichia coli only if the host RecBCD enzyme, or a related helicase-nuclease, is inhibited or genetically inactivated, has been developed. In embodiments described herein, this screen has been optimized for use in 1536-well plates and many small molecules have been screened as inhibiting the Helicobacter pylori AddAB enzyme expressed in an E. coli recBCD deletion strain.

Secondary screening utilized assays with cells expressing AddAB or RecBCD and a viability assay that measured the effect of compounds on cell growth without phage infection. From this screening campaign, a subset of compounds exhibiting efficacy and selectivity were tested for inhibition of purified AddAB and RecBCD helicase and nuclease activities and in cell-based assays for recombination; several were active in the 0.1-50 μM range in at least one assay. Compounds structurally related to two of these were similarly tested, and compounds active in the 0.1-50 μM range were identified.

Development of the Screens

Because phage T4 gene 2 mutants grow in E. coli mutants lacking RecBCD nuclease activity but not in E. coli wild-type or other nuclease-deficient mutants (Oliver, D. B.; Goldberg, E. B., J. Mol. Biol. 1977, 116, 877-881), RecBCD evidently is the only nuclease that blocks this mutant phage's growth. Wild-type T4 phage are able to grow in wild-type E. coli presumably because the gene 2 protein binds to the ends of the ds DNA in the phage virions and, upon injection of the DNA into the host cell, blocks the action of RecBCD (FIG. 1). As such, conditions were developed that allow E. coli recB21 null mutants but not recBCD⁺ cells to be lysed by T4 gene 2 mutant phage.

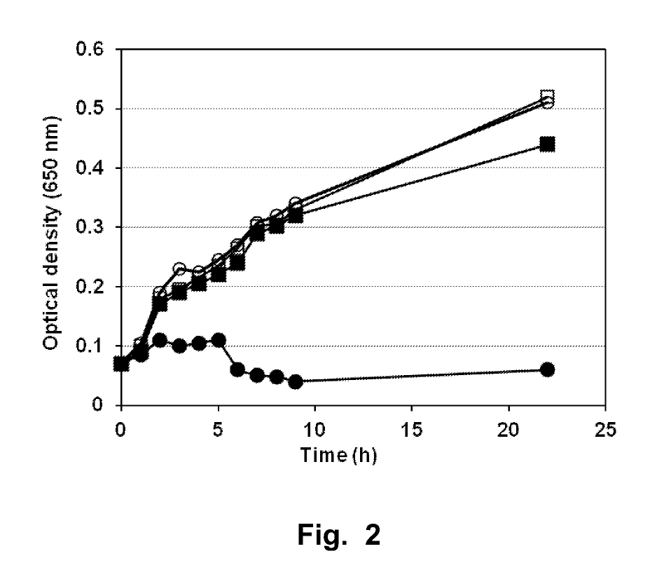

After infection at a multiplicity of infection (MOI) of 0.01 in liquid culture, E. coli recB21 cells increase in OD for about 2 h and then cease growth, presumably when the phage have multiplied sufficiently to infect and begin to lyse most or all of the cells (FIG. 2). Under these conditions, recBCD⁺ cells grow about the same with or without phage infection. E. coli cells bearing a deletion of the recBCD genes and harboring a plasmid expressing the H. pylori addAB⁺ genes also grow about the same with or without phage infection (Amundsen, S. K.; Fero, J.; Hansen, L. M.; Cromie, G. A.; Solnick, J. V.; Smith, G. R.; Salama, N. R., Molec. Microb. 2008, 69, 994-1007.). An inhibitor of RecBCD or AddAB nuclease would thus likely allow T4 gene 2 mutant phage to block the growth of recBCD⁺ (or addAB⁺) cells, and an inhibitor specific for RecBCD or AddAB would not block the growth of uninfected cells. These criteria were used to screen for specific inhibitors of these two enzymes.

To screen large numbers of compounds in 0.1 ml cultures in 96-well plates, reproducible results were obtained by diluting freshly grown cells about 1:100 into LB broth containing compound and adding phage after 1 h of incubation. In every well recB21 cells were lysed or failed to grow and in nearly all of the wells recBCD⁺ cells grew to at least as high OD as without infection (e.g., FIG. 2); however, in about 2% of the wells recBCD⁺ cells were also lysed. Similar results were found with E. coli expressing H. pylori AddAB. The wells with lysed cells were found to contain revertants or pseudorevertants of the gene 2 mutation, a nonsense mutation at codon 247 of 275 codons in the gene (NCBI NP_049754) (Miller, E. S.; Kutter, E.; Mosig, G.; Arisaka, F.; Kunisawa, T.; Ruger, W., Microbiol. Molec. Biol. Rev. 2003, 67 (1), 86-156). To circumvent this problem, a phage was constructed with three nonsense mutations, at codons 247, 248, and 249. A phage with the deletion of gene 2 was unable to be constructed, presumably because part of the gene 2 protein is also required for packaging phage DNA (Wang, G. R.; Vianelli, A.; Goldberg, E. B., J. Bacteriol. 2000, 182 (3), 672-9). Revertants of this triple nonsense phage, designated gene 2 am149, have not been observed in >10³ assays without a compound added.

Screening of Large Libraries

To screen larger libraries, the assay was converted for use in 1536-well plates. The AddAB (strain V3605) phage assay was selected as the primary screen to test a total of 326,100 distinct chemical entities. All compounds were tested at 12 μM in singlicate. Primary screen results were reviewed, and 937 compounds that appeared nominally active (“hits”) were advanced for secondary assay analysis; 52 of these compounds were unavailable from the NIH Molecular Libraries-Small Molecule Repository.

In the secondary assays, the 885 available compounds were first retested in triplicate in the primary screening assay to confirm activity. The same compounds were also tested in triplicate for their effect on the viability of strain V3065 (i.e., in the absence of phage) and also screened in triplicate in strain V66 in the presence of phage to determine RecBCD inhibition. From these efforts, 225 hits that appeared active in either the RecBCD or AddAB inhibition assays were advanced to titration assays.

In titration assays, compounds were tested in triplicate as 10-point titrations using the same protocols used for secondary assays: IC₅₀ values were then determined. All HTS assays were determined to be robust, as each assay demonstrated Z′-scores greater than 0.8.

A summary of the entire screening effort, including summary statistics for all screening assays, is presented in Example 1.

Direct Enzyme Assays

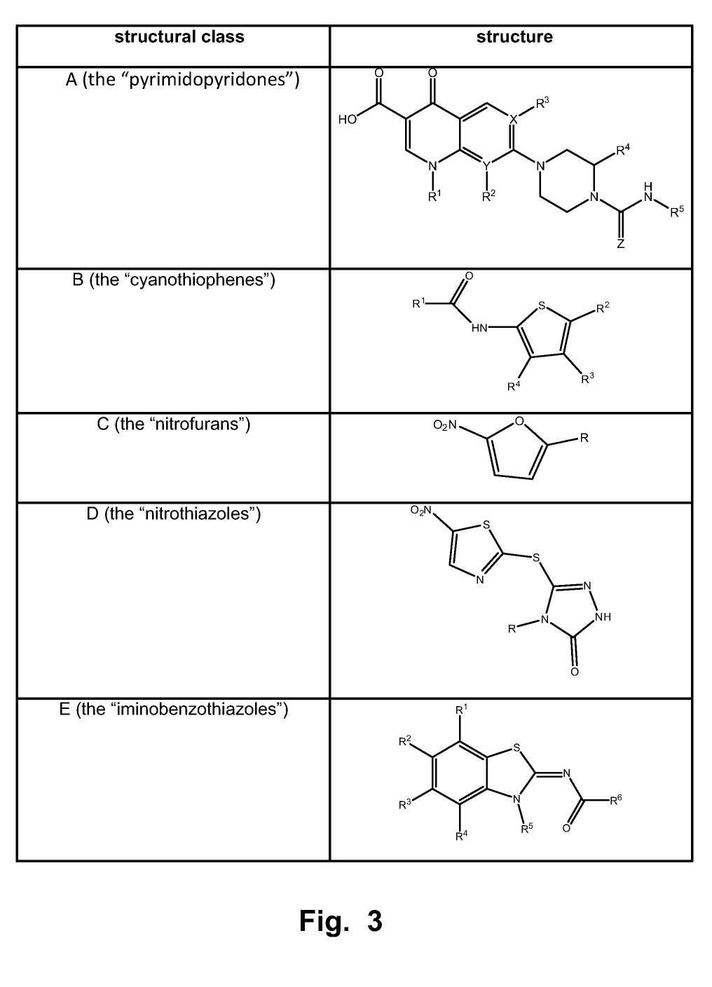

From the screens above, seven compounds (Example 1) and five related compounds were chosen for direct tests with purified enzymes. These compounds, listed in Table 9 and shown generally in FIG. 3, form four general structural classes, designated here “pyrimidopyridones” (group A), “cyanothiophenes” (group B), “nitrofurans” (group C), and “nitrothiazole” (group D). An additional fifth class, the “iminobenzothiazoles” (group E) was also identified.

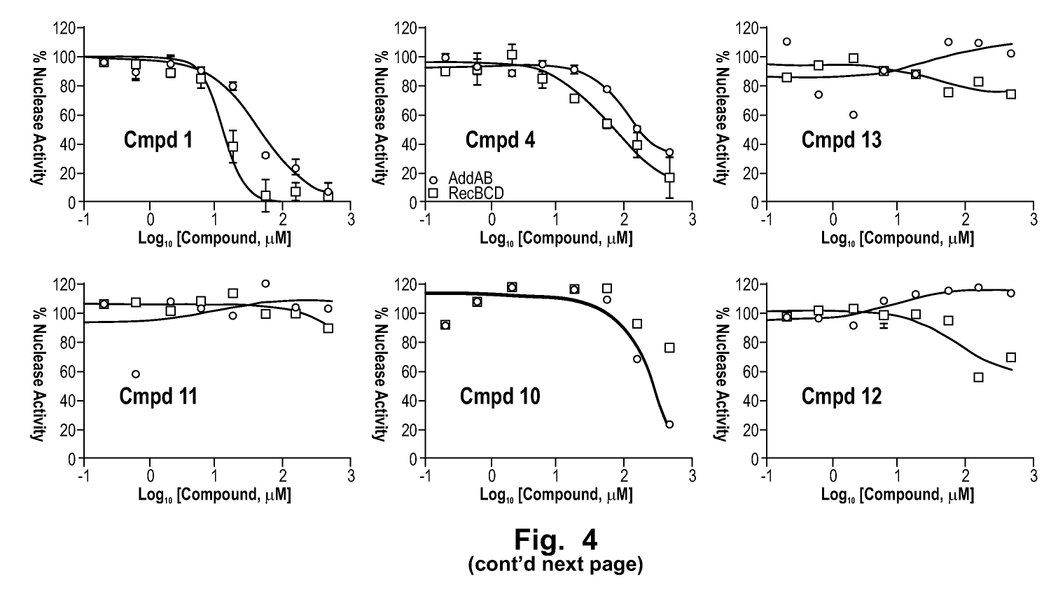

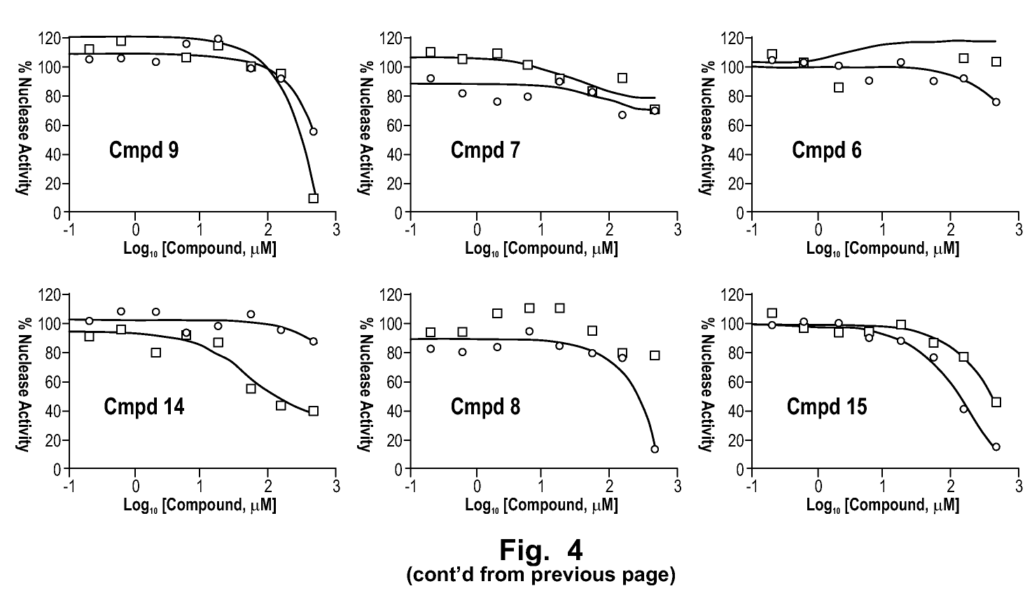

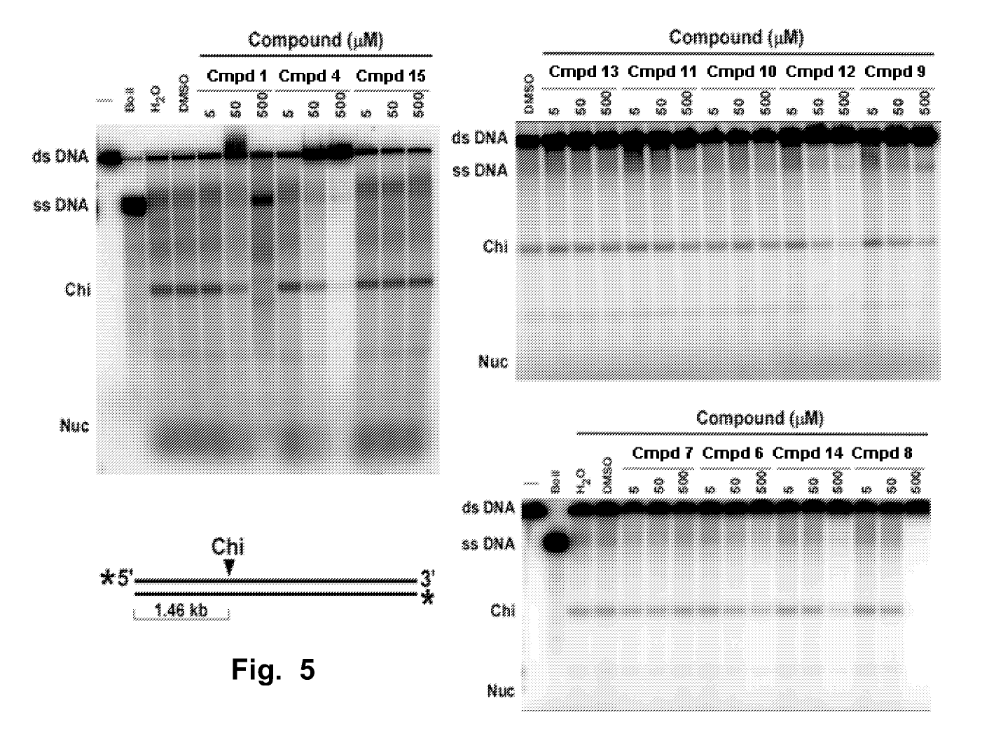

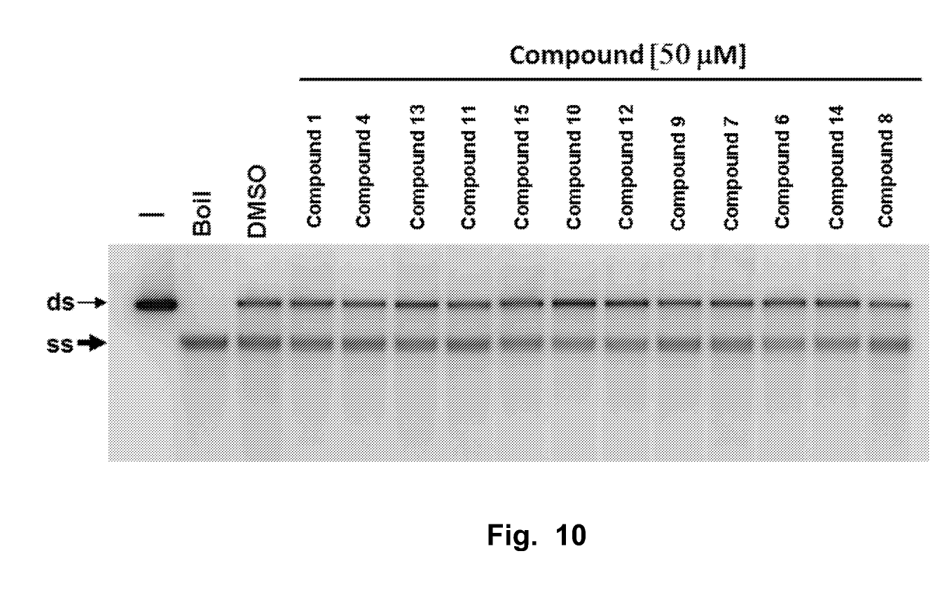

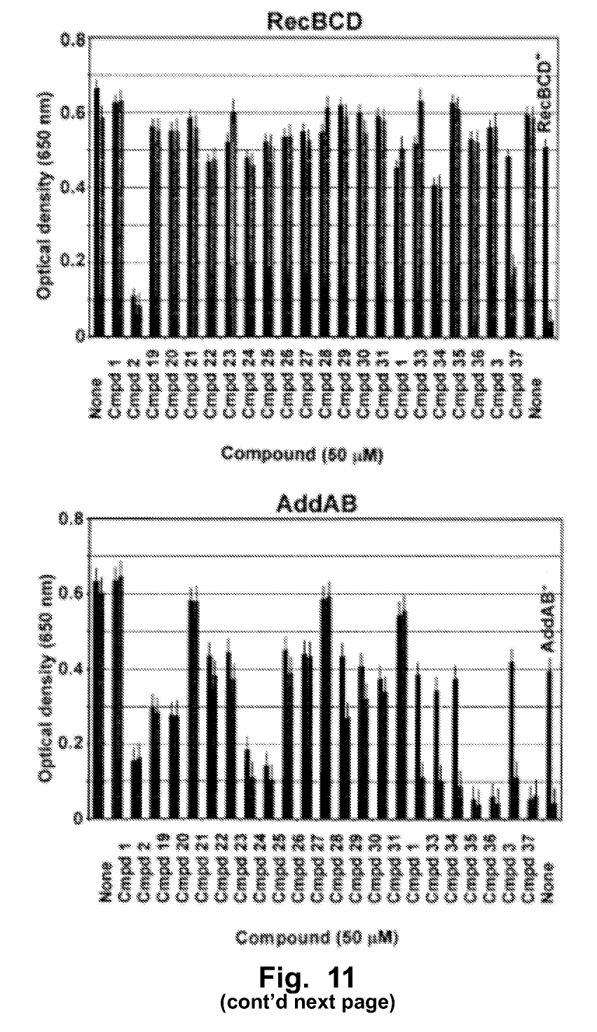

The ability of the compounds to inhibit the exonuclease activity of purified E. coli RecBCD and H. pylori AddAB enzymes was assayed initially. Compound concentrations from 0.2 μM to 500 μM were tested. IC₅₀ values ranged from about 15 μM to over 100 μM (FIG. 4 and Table 9). For both enzymes, Compound 1 (group A) and Compound 4 (group B) were potent. In helicase assays, these compounds did not significantly inhibit AddAB (FIG. 10), but several inhibited RecBCD's helicase and Chi-cutting activities. Compound 4 inhibited both of these activities with an IC₅₀ of about 20 μM (FIG. 5), and three nitrofurans (Compounds 12, 14, and 8) inhibited with an IC₅₀ of <500 μM. Compound 1 appeared to inhibit in a biphasic manner, by inhibiting both helicase and Chi-cutting activities at 50 μM, but at 500 μM it appeared to stimulate the helicase and to change the position of specific cuts.

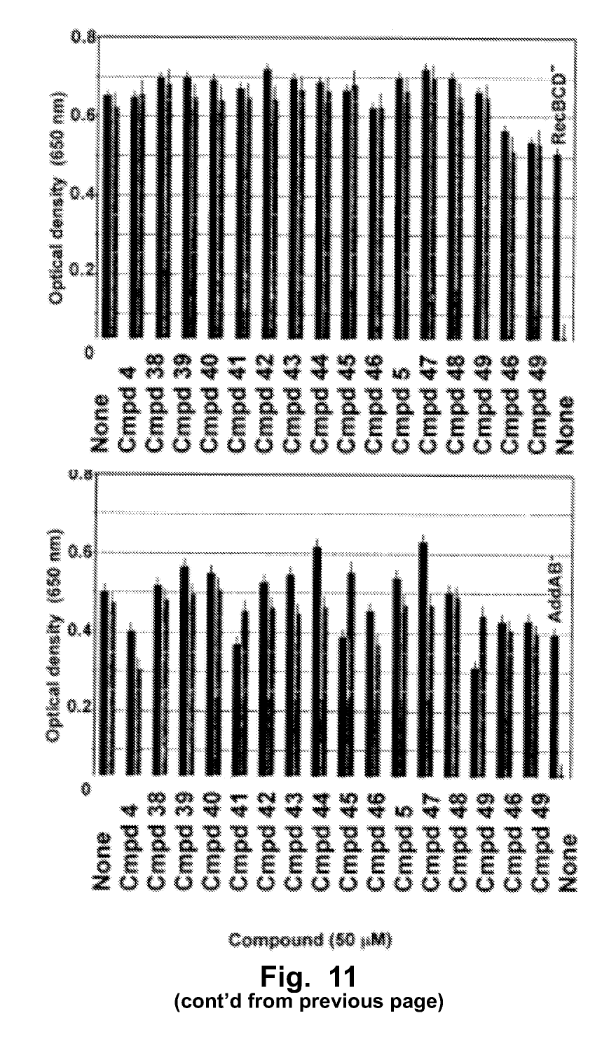

To identify additional compounds, compounds related to Compound 1 and Compound 4 were tested. In the T4 gene 2 mutant-sensitization assay, used for the initial screen, Compound 3 appeared to be RecBCD-specific and Compounds 34, 35, 36, and 39 appeared AddAB-specific; i.e., growth of cells with each enzyme was inhibited only if cells were infected with T4 gene 2⁻ phage (FIG. 11). Compound 2 inhibited growth of cells with either enzyme even without phage infection; thus, this compound appears to inhibit E. coli growth independently of RecBCD or AddAB. Compounds 25, 26, 37, 38, and 40, and to a lesser extent Compounds 20 and 21, also inhibited growth without phage infection, but only of cells with AddAB. This result may reflect the poorer growth of E. coli with AddAB than with RecBCD, perhaps because AddAB does not effectively utilize E. coli RecA but RecBCD does (Amundsen, S. K.; Fero, J.; Salama, N. R.; Smith, G. R., J. Biol. Chem. 2009, 284, 16759-66).

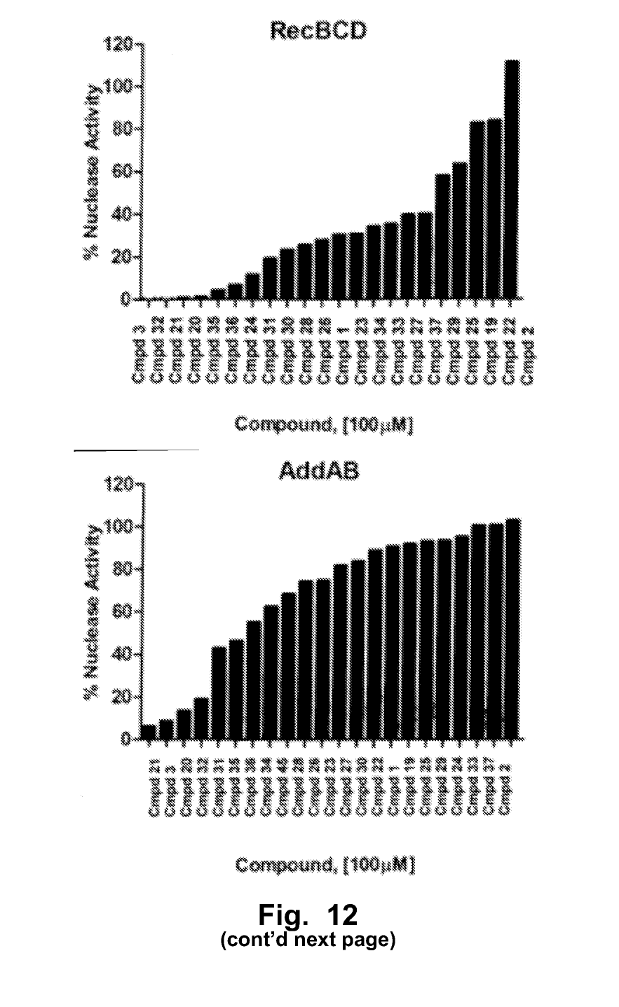

In direct tests of inhibition of the purified enzymes, it was found that most of the derivatives of Compound 1 inhibited RecBCD nuclease activity and several inhibited AddAB nuclease activity (FIG. 12). Notably, Compound 3 inhibited both enzymes in both cell-based and enzyme-based assays. Compound 2 did not inhibit either purified enzyme, a result consistent with its inhibition of cell growth independent of RecBCD or AddAB (FIG. 11). Such results serve to validate the cell-based screen.

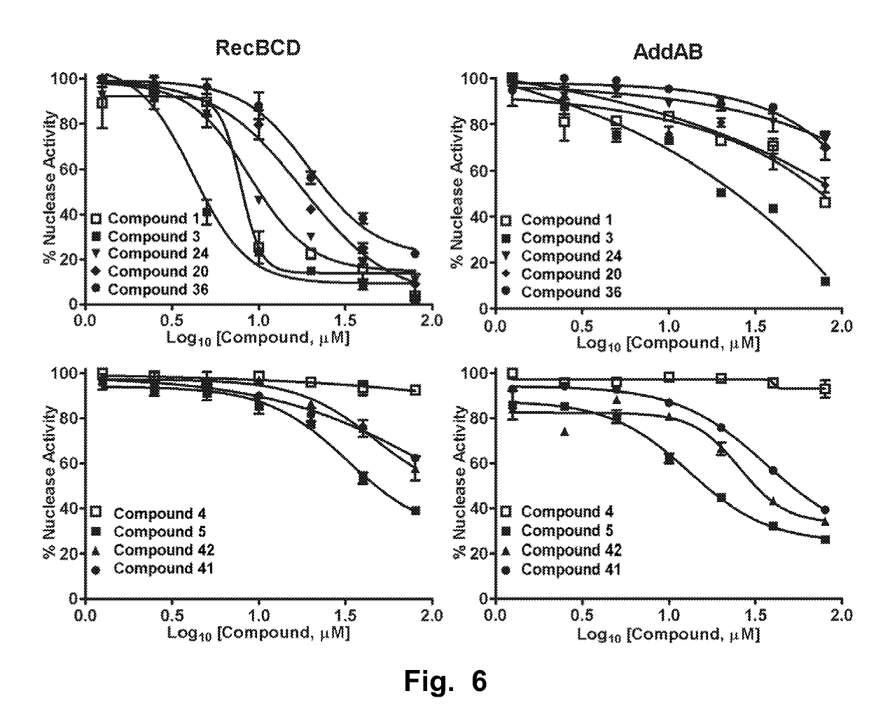

Derivatives of Compound 4, e.g., Compounds 5, 44, and 45, inhibited the nuclease activity of both of the purified enzymes with IC₅₀ of <100 μM (FIGS. 6 and 12), From these nuclease assays, Compounds 3 and 5 appear to be potent inhibitors. The IC₅₀ of Compound 3 is about 5 μM for RecBCD and about 25 μM for AddAB, and that of Compound 5 is about 30 μM for H. pylori RecBCD and about 15 μM for H. pylori AddAB (FIG. 6).

Assays of the RecBCD helicase and Chi-cutting activities showed that Compound 3 and Compound 5 inhibited these activities much like the parent compounds: Compound 3 inhibited in a biphasic way and Compound 5 in a monophasic way (IC₅₀ of about 5 and about 30 μM, respectively); neither compound inhibited AddAB unwinding activity.

Inhibition of Intracellular Recombination

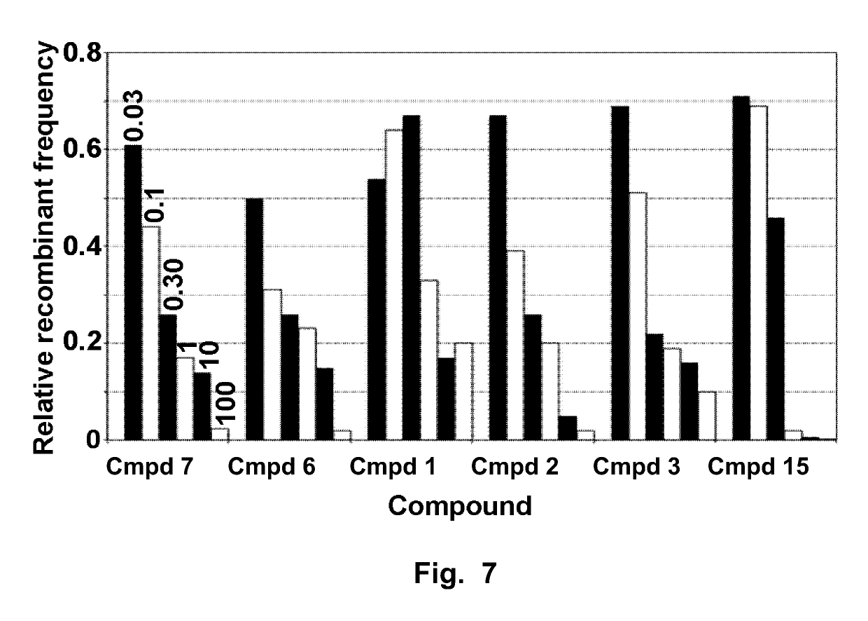

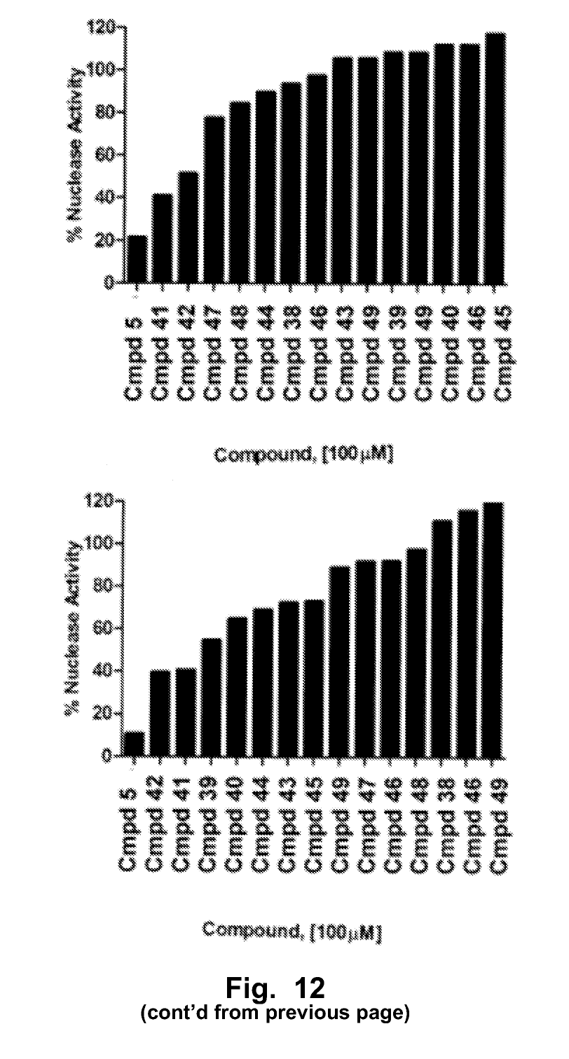

To explore further the ability of the assayed compounds to inhibit RecBCD or AddAB in cells, the ability of the compounds to inhibit E. coli Hfr-based recombination and phage λ recombination was analyzed. When tested at 100 or 200 μM, seven of the initial 12 compounds reduced Hfr recombinant frequencies by less than a factor of 2, but the remaining five compounds and four of the derivatives inhibited more, by factors up to about 200 (Table 9 and FIGS. 7 and 13). For six of these latter nine, dose-response assays showed that Compounds 1, 2, 3, 6, 7, and 15 inhibited with IC₅₀ of <1 μM (FIG. 7).

For Compounds 2, 6, and 7 this outcome was surprising, since these compounds did not significantly affect RecBCD nuclease, unwinding or Chi-cutting activities (FIGS. 4, 5, and 12). These compounds at ≧40 μM inhibit cell growth without T4 gene 2 mutant infection (FIG. 11; Table 9) and may inhibit an enzyme other than RecBCD required for recombination. For example, DNA gyrase, which is required for RecBCD pathway recombination (Ennis, D. G.; Amundsen, S. K.; Smith, G. R., Genetics 1987, 115, 11-24) is inhibited by pyrimidopyridones such as Compound 2 (pipemidic acid) (Zweerink, M. M.; Edison, A., Antimicrob. Agents Chemother. 1986, 29 (4), 598-601; Shen, L. L.; Pernet, A. G., Proc. Natl. Acad. Sci. USA 1985, 82 (2), 307-11). In contrast Compounds 1, 3, and 15 inhibited Hfr recombination with IC₅₀ of <1 μM (FIG. 7), as well as RecBCD nuclease, unwinding, or Chi-cutting activities, albeit at higher IC₅₀ (FIGS. 4, 5, 6, and 12). Compound 1 also inhibited Hfr recombination when AddAB replaced RecBCD in E. coli cells: 20 μM Compound 1 reduced the recombinant frequency to that of cells lacking AddAB or RecBCD (0.02% His⁺ Str^{R} recombinants per Hfr donor cell).

To determine if inhibition of Hfr recombination was specific to RecBCD, the six compounds used in FIG. 7 for inhibition of Hfr recombination were tested by two alternative pathways, called RecE and RecF, that are activated by mutations (sbc) that suppress the recombination-deficiency of recB recC null mutants (Clark, A. J., Annu. Rev. Genet. 1973, 7, 67-86). Each compound inhibited recombination by the RecBCD (wild-type) pathway to a greater extent than recombination by the RecE or RecF pathway (Table 10). Two compounds, Compounds 2 and 15, significantly inhibited recombination by the latter pathways, by factors of 6-13.

Thus, these data suggest that these six compounds are not specific to RecBCD enzyme, but they may inhibit instead, or in addition, an enzyme required more stringently by the RecBCD pathway than by the other pathways. For example, DNA gyrase, which is inhibited by Compound 2 (Zweerink, M. M.; Edison, A., Antimicrob. Agents Chemother. 1986, 29 (4), 598-601; Shen, L. L.; Pernet, A. G., Proc. Natl. Acad. Sci. USA 1985, 82 (2), 307-11), may be the target of Compound 2, as suggested above. The effects of Compounds 1, 3, and 6 on purified RecBCD (FIGS. 4, 5, 6, and 12) and on intracellular recombination (Tables 9 and 10 and FIGS. 7 and 13) indicate that at least these three compounds inhibit recombination by RecBCD in cells.

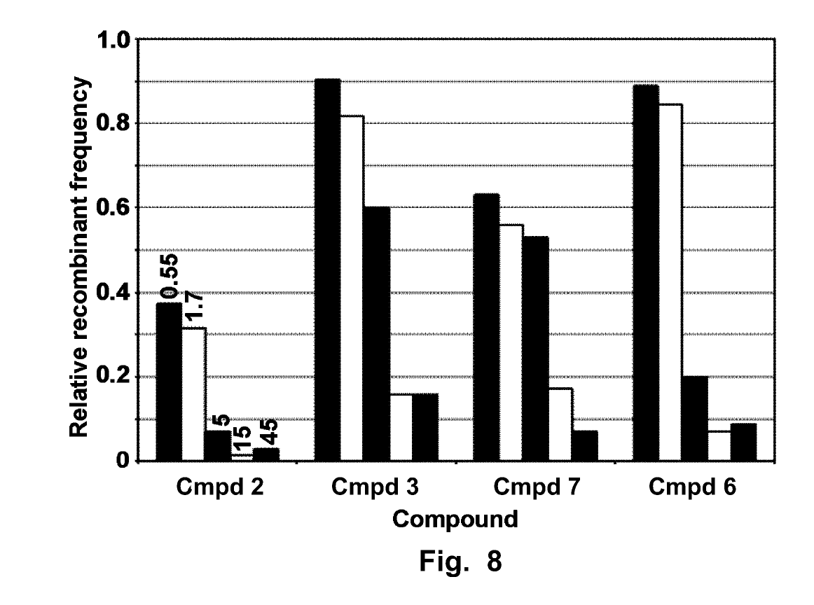

To extend the intracellular assays, the effects of the compounds on phage A recombination dependent on RecBCD (the phages are red gam mutants) were analyzed. Results were obtained with Compounds 7, 6, 2, and 3, which inhibited A recombination with IC₅₀ of <15, 5, 0.6, and 5 μM, respectively (FIG. 8). These compounds also inhibited RecBCD-dependent Hfr recombination, as noted above. In λ crosses, the activity of the Chi hotspot, which regulates RecBCD activity (Smith, G. R., Microbiol Mol Biol Rev 2012, 76, 217-228; Dillingham, M. S.; Kowalczykowski, S. C., Microbiol Mol Biol Rev 2008, 72 (4), 642-71; Smith, G. R., Annu. Rev. Genet. 2001, 35, 243-274), is measured as the ratio of the recombinant frequency in a genetic interval with Chi to that in the same interval without Chi. This ratio is about 5 in wild-type E. coli and 1 in recBCD null mutants, meaning that Chi is inactive in the absence of RecBCD (Stahl, F. W.; Stahl, M. M., Genetics 1977, 86, 715-725). Three compounds, Compounds 2, 3, and 15, significantly lowered Chi hotspot activity to a value of about 3 (Table 9).

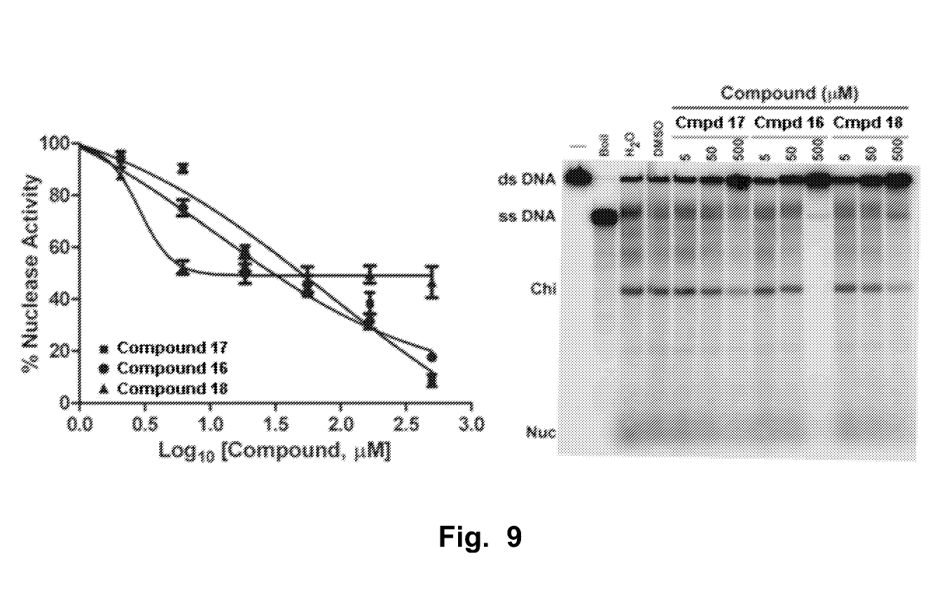

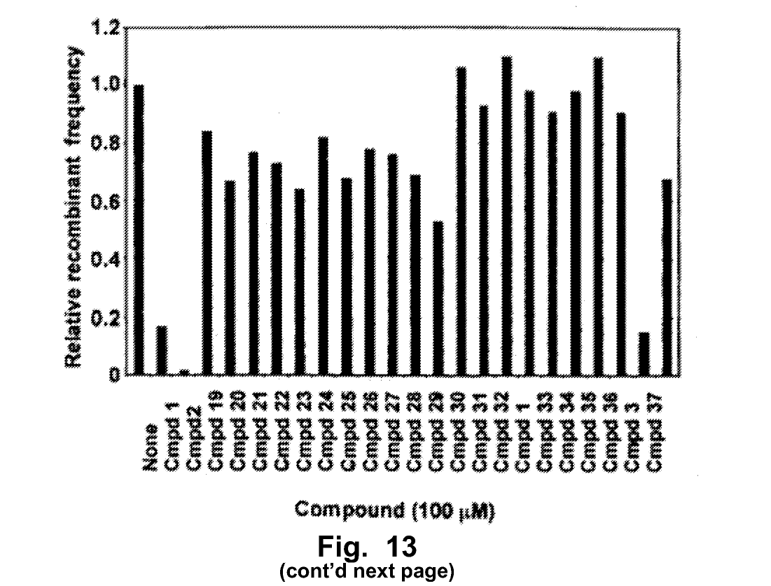

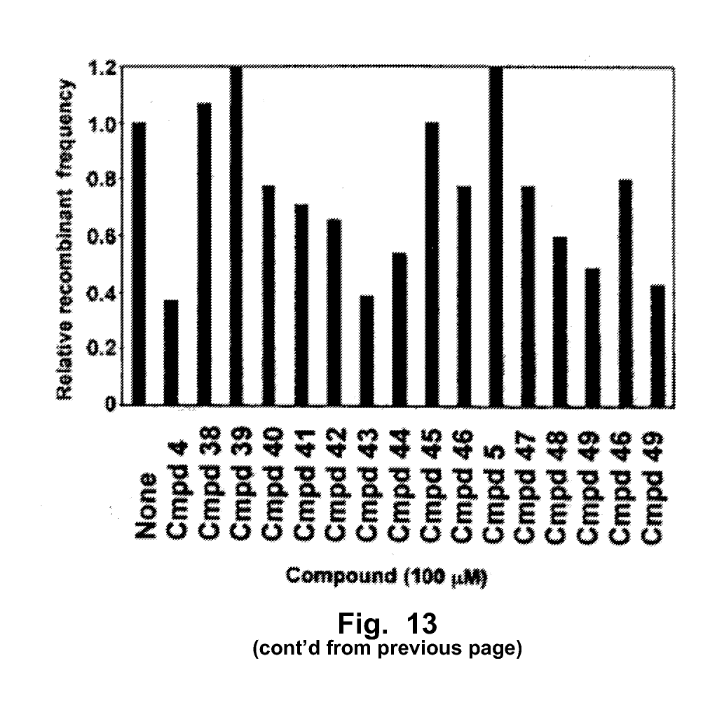

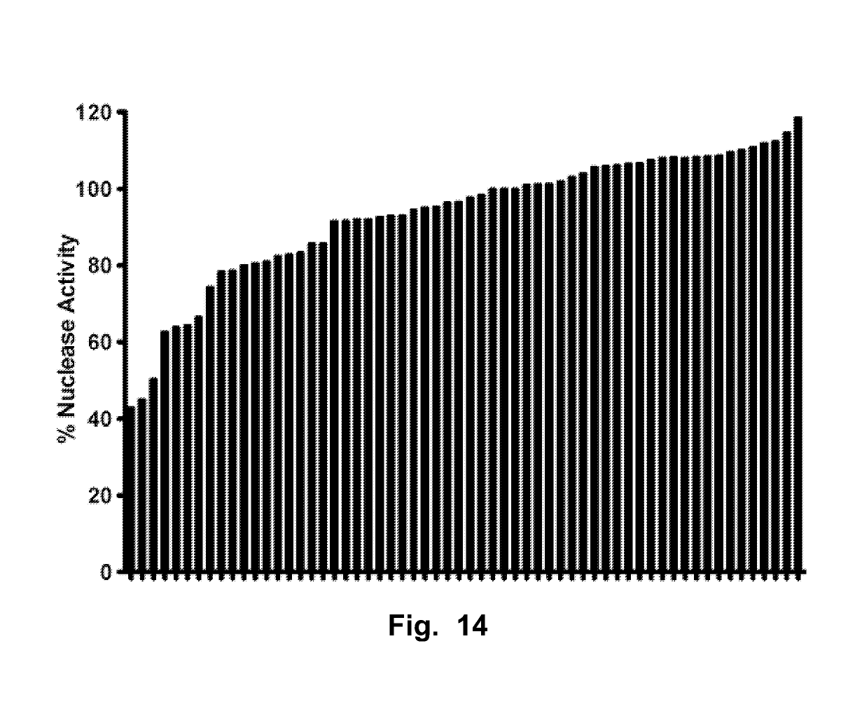

A class of compounds, here called “iminobenzothiazoles” or class E, was identified and tested for inhibition of E. coli RecBCD nuclease activity, and three were identified with an IC₅₀ of <100 μM (FIGS. 9 and 14). Their dose responses were unexpected. Compound 18 detectably inhibited at concentrations as low as about 2 μM; inhibition was about 50% at about 5 μM and remained at that level at concentrations as high as 500 μM. As their concentrations were raised, Compounds 16 and 17 inhibited more gradually than expected for single-site inhibition: activity decreased from about 90% to about 10% over a range of about 2.5 log₁₀.

These results may reflect differential inhibition of the two helicases in RecBCD. Without being bound by theory, Compound 18 may inhibit only one of the helicases, with IC₅₀ of about 2 μM, and this helicase may be responsible for only half of the nuclease activity measured. The other two compounds may inhibit this helicase with IC₅₀ of about 10 μM and the other helicase with IC₅₀ of about 100 μM, so that nuclease activity is inhibited only gradually as the concentration is raised. DNA unwinding and Chi-cutting activities were also inhibited by these three compounds (FIG. 9). The IC₅₀ values, most readily quantified for Chi cutting, were about 50 μM.

Advantages of a Cell-Based Screen

By using a cell-based assay, only compounds that entered E. coli cells sufficiently readily to inhibit the target enzyme, either the native RecBCD enzyme or the AddAB enzyme expressed from the H. pylori genes, or RecBCD or AddAB expressed from genes of any species, are able to be analyzed. In addition, these compounds must inhibit the enzyme in its natural environment, which might be markedly different from that of conditions normally employed to study the purified enzymes.

The assay disclosed herein is simple and inexpensive, since it employs only bacteria and phage, which are readily grown in large quantities, and reliable: Z′ factors of ˜0.9 were routinely observed (Table 8). In principle this assay is specific for RecBCD or related nucleases, such as AddAB, since activity of the critical reagent used—phage T4 gene 2 mutants' lysis of E. coli cells (FIG. 1)—is detected only in recBCD mutants (Oliver, D. B.; Goldberg, E. B., J. Mol. Biol. 1977, 116, 877-881). Compounds that inhibit AddAB or RecBCD or both were identified. Certain compounds, Compound 1 (a “pyrimidopyridone”) and Compound 4 (a “cyanothiophene”), for example, were found to inhibit the ds exonuclease, DNA unwinding, and Chi-cutting activities of RecBCD and the ds exonuclease activity of AddAB (FIGS. 4, 5, 6, and 12). Thus, this screen indeed revealed compounds of the desired type.

Identification of Potent Inhibitors of AddAB and RecBCD Enzymes

By direct nuclease assays in the presence of about 20 compounds structurally related to Compounds 1 and 4, additional inhibitors were identified. Compound 5 inhibits AddAB nuclease with an IC₅₀ of about 15 μM, but like its parent compound (Compound 4), it does not detectably inhibit AddAB unwinding activity under the conditions used (FIGS. 6, 10, and 12).

Like its parent compound (Compound 1), Compound 3 inhibited all of the activities of RecBCD tested, both with purified enzyme and with cell-based recombination assays (FIGS. 4, 5, 6, 7, 8, 12, and 13; Tables 9 and 10). IC₅₀ values were about 3 μM for nuclease and Chi-cutting with purified enzyme, about 0.3 μM for Hfr recombination and about 5 μM for phage λ recombination promoted by RecBCD; it also significantly reduced Chi hotspot activity in λ crosses (Table 9). It only marginally inhibits recombination by the E. coli RecE and RecF pathways, which do not employ RecBCD (Table 10). In the T4 gene 2 mutant screen, it only slightly inhibits E. coli growth in the absence of phage but strongly inhibits in the presence of phage (FIG. 11).

The IC₅₀ values of Compounds 1 and 3 were about 10 times lower in the intracellular assays for Hfr recombination than in assays with purified enzyme. Without being bound by theory, this result may reflect differences in the enzyme's environment during the assays, or it may reflect some activity of RecBCD not yet assayed, such as the loading of RecA protein after action at Chi (Anderson, D. G.; Kowalczykowski, S. C., Cell 1997, 90, 77-86), that is even more sensitive to inhibition than the nuclease, DNA unwinding, and Chi-cutting. This result suggests that these or related compounds may be effective as antibacterial drugs.

The biphasic dose-response curve for inhibition of DNA unwinding and Chi-cutting by Compound 1 suggests a complex interaction between this compound and the RecBCD enzyme. At about 50 μM compound, unwinding and Chi-cutting were strongly inhibited, but at about 500 μM, the unwinding appeared to be stimulated and DNA was cut at novel positions (FIG. 5). Since RecBCD has two helicases and a nuclease involved in these activities, the compound may have differential effects on two or more primary activities. For example, one helicase may be simply inhibited at low concentrations, and the other stimulated or altered at high concentrations. The cutting of DNA at novel positions is reminiscent of the behavior of RecBCD enzyme from two mutants altered in the RecB helicase domain, recB2732 (Y803H) and recB2734 (V804E). These mutant enzymes cut not at Chi but at a position that depends on the length of the DNA substrate (Amundsen, S. K.; Taylor, A. F.; Reddy, M.; Smith, G. R., Genes Dev 2007, 21 (24), 3296-307). Without being bound by a particular theory, an hypothesis is that the RecB nuclease cuts wherever it is on the DNA when the faster helicase, RecD, reaches the end of the substrate, and that the recB mutations sensitize the enzyme to a signal from Chi through RecC to stop RecD when RecBCD encounters Chi. Compound 1 may similarly sensitize the enzyme to signaling between RecD and RecB.

Searching for additional effective inhibitors related to Compound 1, Compound 2 was tested, which has the pyrimidopyridone part but not the benzene ring part of Compound 1 in general structure A (FIG. 3). Compound 2 did not detectably inhibit the nuclease activity of purified AddAB or RecBCD enzyme (FIG. 12), but it strongly inhibited Hfr and phage λ recombination (FIGS. 7, 8, and 13). In the T4 gene 2 mutant assay, it inhibited E. coli growth with or without addition of phage (FIG. 11). These results indicate that Compound 2 inhibits some cellular component other than RecBCD, such as DNA gyrase, which is known to be inhibited by Compound 2 (pipemidic acid) (Zweerink, M. M.; Edison, A., Antimicrob. Agents Chemother. 1986, 29 (4), 598-601; Shen, L. L.; Pernet, A. G., Proc. Natl. Acad. Sci. USA 1985, 82 (2), 307-11) and is required for cell growth (Gottesman, M. M.; Hicks, M. L.; Gellert, M., J. Mol. Biol. 1974, 77, 531-547) and for recombination (Ennis, D. G.; Amundsen, S. K.; Smith, G. R., Genetics 1987, 115, 11-24). These results suggest, in turn, that the benzene ring part of Compound 1 may function to inhibit RecBCD. Indeed, Compound 3, the m-trifluoromethyl isomer of Compound 1, is more potent than the o-trifluoromethyl parent compound in assays of both RecBCD and AddAB nuclease (FIG. 6).

### V. Methods of Use

Methods for using the compounds and compositions described herein are described. In some embodiments, such methods include treating a bacterial infection by inhibition of bacterial DNA repair enzymes, including AddAB and RecBCD helicase-nucleases. In other embodiments, such methods include reducing bacterial survival based on inhibition of bacterial DNA repair enzymes, including AddAB and RecBCD helicase-nucleases. Selective inhibitors of bacterial DNA repair enzymes, including AddAB and RecBCD helicase-nucleases, may be useful as and may lessen the evolution of bacteria resistant to the inhibitor, an important goal in current antibacterial drug therapy. In further embodiments, the methods for treating a bacterial infection according to the present description further include inhibiting bacterial DNA gyrase in addition or as an alternative to inhibiting one or more bacterial DNA helicases. Methods for the administration of the compounds and compositions described herein to a subject are also described herein.

The active compounds and compositions of the present invention are useful for treating a subject, including a mammal or other animal, infected with a microorganism, including bacteria. In particular embodiments, the methods described herein include selectively inhibiting one or more bacterial helicases selected from the RecBCD and/or AddAB families of helicases. In further embodiments, the methods described herein include inhibiting bacterial DNA gyrase in addition or as an alternative to inhibiting one or more bacterial helicases as described herein. In embodiments of the methods for using the compounds and compositions described herein to treat a bacterial infection in a subject, a therapeutically effective amount of one or more of the active compounds described herein is administered to the subject.

Methods for treating diseases or disorders associated with microorganisms including bacteria are also provided. In such methods, one or more active compound as described herein is provided and a therapeutically effective amount of the compound is administered to a subject suffering from the bacterial infection. In certain such embodiments, therapeutically effective amounts of two or more active compounds may be administered to the subject. The bacteria-associated disease or disorder treated by methods according to the present description may be selected from any of the bacteria-associated diseases or disorders described herein.

In each embodiment of the methods of use described herein, the active compound(s) used or administered may be selected from those described herein, including any pharmaceutically acceptable salts, esters, isomers or solvates thereof. Moreover, the active compound(s) may be provided and delivered or administered in a pharmaceutical composition according to the present description. In embodiments of the methods described herein, exposure of cells to one or more active compounds or administration of one or more active compounds to a subject includes delivering a pharmaceutical composition as described herein using any suitable route of administration, technique or technology, including those described in association with the pharmaceutical compositions and methods detailed herein.

In some embodiments of the methods of use described herein, the active compounds may be delivered or administered locally to the area in need of treatment. Local administration can be achieved by, for example, and not by way of limitation, local infusion during surgery, topical application (e.g., in conjunction with a wound dressing after surgery), by injection, by means of a catheter, by means of a suppository, or by means of an implant. In another embodiment, the active compound(s) of the invention can be delivered in a vesicle, in particular a liposome (see, e.g., Langer, Science 249:1527-33, 1990; Treat et al, in Liposomes in the Therapy of Infectious Disease and Cancer, Lopez-Berestein and Fidler (eds.), Liss, New York, pp. 353-65, 1989; Lopez-Berestein, supra, pp. 317-27).

In yet other embodiments of the methods of use, active compound(s) and compositions can be delivered in a controlled release system. In one such embodiment, a pump can be used (see, e.g., Langer, supra; Sefton, CRC Crit. Ref. Biomed. Eng. 14:201, 1987; Buchwald et al., Surgery 88:507, 1980; Saudek et al., N. Engl. J. Med. 321:574, 1989). In another such embodiment, a polymeric controlled release system or formulation can be used (see, e.g., Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J. Macro mol. Sci. Rev. Macromol. Chem. 23:61, 1983; see also Levy et al, Science 228: 190, 1985; During et al, Ann. Neurol. 25:351, 1989; Howard et al, J. Neurosurg. 71:105, 1989). In yet another such embodiment, a controlled release system delivering the active compound(s) or composition can be placed in proximity of the therapeutic target, thus requiring a reduced systemic dose (see, e.g., Goodson, Medical Applications of Controlled Release, supra, Vol. 2, pp. 115-138, 1984). Other controlled release systems are discussed in, for example, the review by Langer (Science 249: 1527-1533, 1990).

The practice of the present invention employs, unless otherwise indicated, conventional techniques of chemistry, biochemistry, molecular biology, microbiology, recombinant DNA, genetics, immunology, cell biology, cell culture and transgenic biology, which are within the skill of the art.

The following examples are provided merely as illustrative of various aspects of the invention and shall not be construed to limit the invention. It is to be understood that the disclosed compositions and methods are not limited to the particular methodologies, protocols, and reagents described herein. In each instance, unless otherwise specified, standard materials and methods were used in carrying out the work described in the Examples provided.

### EXAMPLES

### Example 1

Compounds: For a 1536-well primary screen, the Molecular Libraries Small Molecule Repository (MLSMR) library was provided by the NIH's Roadmap: Molecular Libraries Initiative. The MLSMR library is a highly diversified collection of small molecules (more that 50% of compounds exhibit molecular weights between 350 and 410 g/mol) comprising both synthetic and natural products. Powders of compounds in Table 9 were obtained from ChemBridge Laboratories, except for Compounds 2, 3, and 7 (Vitas-M lab); Compounds 11 and 13 (Enamine); Compound 15 (Maybridge); Compound 12 (ChemDiv); and Compounds 16, 17 and 18 (Life Chemicals, Burlington, Canada). An additional library of about 18,400 compounds from Life Chemicals was obtained from Kineta, Inc. (Seattle, Wash.).

Results of the screening, including assay statistics, are summarized in Tables 8-10, below.

TABLE 8
Initial Screening Summary.

| | | | no. of | | no. of | assay | |
|---|---|---|---|---|---|---|---|
| | | | cmpds | | selected | statistics | |
| step | screen type | target | tested | selection criteria | cmpds | Z′ | S/B |
| 1 | primary | AddAB | 326, 100 | Inhibition >12.16%^{a} | 937 | 0.91 ± 0.02 | 3.60 ± 0.15 |
| | screen | | | | | | |
| 2 | confirmation | AddAB | 885 | Inhibition >12.16%^{b} | 256 | 0.84 ± 0.02 | 2.55 ± 0.07 |
| 3 | counter- | RecBCD | 885 | Inhibition >3.95%^{c} | NA^{e} | 0.93 ± 0.02 | 3.73 ± 0.32 |
| | screen | | | | | | |
| 3b | counter- | E. coli | 885 | Inhibition >24.60%^{d} | NA^{e} | 0.88 ± 0.31 | 2.58 ± 0.09 |
| | screen | V3065 | | | | | |
| 4a | titration | AddAB | 225 | IC₅₀ <10 μM for AddAB | 7 | 0.91 ± 0.01 | 2.61 ± 0.06 |
| 4b | selectivity | RecBCD | | and >10 μM for RecBCD | | 0.93 ± 0.01 | 3.46 ± 0.07 |
| 4c | cytotoxicity | E. coli | | and E. coli V3065 | | 0.89 ± 0.02 | 2.58 ± 0.06 |
| | | V3065 | | | | | |

Notes for Table 8: ^{a} The primary screen hit cutoff was calculated at the average percent inhibition of all test compounds plus three times the standard deviation; ^{b} The hit cutoff calculated for the primary run was also applied to the confirmation run; ^{c} The hit cutoff calculated for the counterscreen was derived from the average percent inhibition of all DMSO-only wells tested plus three times the standard deviation; ^{d} The hit cutoff calculated for the counterscreen was derived from the average percent inhibition of all DMSO-only wells tested plus eight times the standard deviation; and ^{e} NA, not applicable.

TABLE 9
Properties of Active Compounds Found in Screens of AddAB and RecBCD.

| | Inhibition (approximate IC₅₀, μM) | | | | | Relative recombinant | | |
|---|---|---|---|---|---|---|---|---|
| | E. coli | | E. coli | | | frequency^{b} | | Chi |
| | (paddAB) | | recBCD⁺ | | Inhibition | E. coli Hfr | Phage λ | hotspot^{c} |
| | growth | | growth | | of RecBCD | recombination | recombination | activity |
| Cmpd | with T4 | AddAB | with T4 | RecBCD | Chi | at 200 μM | at 200 μM | at 200 μM |
| No. | 2⁻ phage | nuclease | 2⁻ phage | nuclease | cutting | compound | compound | compound |
| 1 | 2.5 | 34 | >40 | 13 | 10 | 16, 15 | 9, 14 | 4.1, 4.6 |
| 2 | ND^{e} | >100 | ND | >100 | ND | 0.2, 0.4 | 9, 11 | 3.1, 3.5 |
| 3 | ND | 26 | ND | 5.1 | ND | 20, 10 | 18, 23 | 2.5, 2.1 |
| 4 | 18 | 96 | >120 | 51 | 20 | 37, 21 | ND | ND |
| 5 | ND | 13 | ND | 33 | >500 | 118, 121^{d} | ND | ND |
| 6 | 8.2 | >100 | >40 | >100 | >100 | 7, 5 | 0.4, 0.3 | 5.3, 5.2 |
| 7 | 9.7 | >100 | 86 | >100 | >500 | 6, 7 | 5, 6 | 4.9, 3.8 |
| 8 | 10 | >100 | >120 | >100 | 100 | 34, 42 | ND | ND |
| 9 | 4.9 | >100 | >120 | >100 | 50 | 37, 43 | ND | ND |
| 10 | >13 | >100 | >120 | >100 | >500 | 47, 51 | ND | ND |
| 11 | 4.8 | >100 | >120 | >100 | >500 | 65, 73 | ND | ND |
| 12 | 17 | >100 | >120 | >100 | 20 | 47, 55 | ND | ND |
| 13 | 21 | >100 | >120 | >100 | >500 | 49, 63 | ND | ND |
| 14 | ND | >100 | ND | >100 | 200 | 49, 54 | ND | ND |
| 15 | 12 | 130 | >120 | >100 | >500 | 0.6, 0.3 | 90, 50 | 3.1, 2.9 |
| 16 | ND | ND | >100 | 10 | 50 | 64, 72^{d} | ND | ND |
| 17 | ND | ND | >100 | 80 | 50 | 78, 67^{d} | ND | ND |
| 18 | ND | ND | ND | 3 | 50 | 57, 47^{d} | ND | ND |

Notes and references for Table 9: (no note for ^{a}), ^{b}Data, from two experiments for each compound, are the recombinant frequencies as a percentage of that without compound; For Hfr recombination these were 8.7 and 8.9% His⁺ Str^{R} per viable Hfr cell in the two experiments, respectively; for λ recombination these were 11.2% and 3.4% J⁺ R⁺, respectively; ^{b}Chi hotspot activity, measured in crosses between λ phages 1081×1082 and 1083×1084 as in ¹, is the frequency of recombinants in an interval with Chi to that in the same interval without Chi; In the absence of compounds, values were 5.1 and 4.9 in the two experiments, respectively; ^{d}Compound was 100 μM; recombinant frequencies in the absence of compound were 7.6% and 8.4%; ^{e}ND, not determined; 1. Stahl, F. W.; Stahl, M. M., Genetics 1977, 86, 715-725.

TABLE 10
Pathway Specificity of Hfr Recombination Inhibitors.

| | | Fold-reduction in recombinant frequency^{a} | | | | | | |
|---|---|---|---|---|---|---|---|---|
| | | Null | | | | | | |
| Pathway | Genotype | mutant^{b} | Cmpd 15 | Cmpd 1 | Cmpd 2 | Cmpd 3 | Cmpd 7 | Cmpd 6 |
| RecBCD | recBCD⁺ | 1000 | 450 | 6.9 | 300 | 9 | 14 | 13 |
| RecF | recB21 | 1000 | 13 | 1.4 | 13 | 1.8 | 1.0 | 1.2 |
| | recC22 | | | | | | | |
| | sbcB14, C(D) | | | | | | | |
| RecE | recB21 | 100 | 11 | 2.0 | 5.6 | 1.8 | 2.7 | 3.1 |
| | recC22 | | | | | | | |
| | sbcA23 | | | | | | | |

Notes and References for Table 10: ^{a}Recipient strains were grown and mated with donor strain V1306 (Hfr P044) in LB plus the indicated compound (100 μM). Data are the mean factor of reduction (n=3) in recombinant frequencies compared to those of the untreated control, which were for V66 (recBCD⁺) 4.5, 10.2, and 11.1%; for JC9387 (recB21 sbcB14 sbcC or D) 1.3, 2.2, 1.9%; and for JC8679 (recB21 sbcA23) 2.2, 2.1, 1.8%. SEM or range was <20% of the mean (n=2 or 3). Strain JC9387 (recB21 sbcB14) presumably also carries an sbcC or sbcD mutation². ^{b} The factor of reduction in the frequency of recombinants observed in crosses between an Hfr donor strain and a recombination-deficient null mutant compared to that in the corresponding rec⁺ parent: for the RecBCD pathway V67 (recB21) compared to V66³; for the RecF pathway JC8111 (recF143) compared to JC9387⁴; for the RecE pathway N2510 (recN262) compared to JC8679.⁵ 2. Lloyd, R. G.; Buckman, C., J. Bacteriol. 1985, 164, 836-844; 3. Schultz, D. W.; Taylor, A. F.; Smith, G. R., J. Bacteriol. 1983, 155, 664-680; 4. Horii, Clark, A. J., J. Mol. Biol. 1973, 80, 327-344; 5. Lloyd, R. G.; Buckman, C.; Benson, F. E., J. Gen. Microbiol. 1987, 133, 2531-2538.

Bacterial and Phage Strains. The E. coli strains used are listed in Table 11, and phage λ strains in Table 12.

TABLE 11
E. coli strains.

| Strain | Genotype | Ref. |
|---|---|---|
| V66 | hisG4 argA21 met recF143 rpsL31 galK2 xyl-5 F⁻ λ⁻ | ¹ |
| V67 | recB21::IS186 hisG4 argA21 met recF143 rpsL31 galK2 xyl-5 F⁻ λ⁻ | ¹ |
| V1306 | thi-1 relA1 λ⁻ (Hfr PO44) | ¹ |
| V2831 | ΔrecBCD2731 <kan> hisG4 met recF143 rpsL31 galK2 xyl-5 F⁻ λ⁻ | ² |
| V3060 | ΔrecBCD2731 <kan> hisG4 met recF143 rpsL31 galK2 xyl-5 F⁻ (λ DE3) | ³ |
| V3065 | ΔrecBCD2731 <kan> hisG4 met recF143 rpsL31 galK2 xyl-5 F⁻ λ⁻ | |
| | (pSA405) | |
| V3069 | ΔrecBCD2731 <kan> hisG4 met recF143 rpsL31 galK2 xyl-5 F⁻ λ⁻ | |
| | (pETDuet-1) | |
| JC8679 | thr-1 leuB6 ara-14 proA2 lacY1 tsx-33 galK2 hisG4 rpsL31 xyl-5 mtl-1 | ⁴ |
| | argE3 thi-1 recB21 recC22 sbcA23 supE44 F⁻ λ⁻ | |
| JC9387 | As JC8679 but sbcA⁺ sbcB15 sbcC (D) sup⁺ F⁻ λ⁻ | ⁴ |
| 594 | lac-3350 galK2 galT22 rpsL179 F⁻ λ⁻ | ⁵ |
| C600 | thr-1 leuB6 thi-1 lacY1 tonA21 supE44 rfbD1 F⁻ λ⁻ | ⁶ |

References for Table 11:
¹Schultz, D. W,; Taylor, A. F.; Smith, G. R., J,. Bacteriol. 1983, 155, 664-680;
²Amundsen, S. K,; Taylor, A. F.; Reddy, M.; Smith, G. R., Genes Dev 2007, 21 (24), 3296-307;
³Amundsen, S. K.; Fero, J.; Hansen, L. M.; Cromie, G. A.; Solnick, J. V.; Smith, G. R.; Salama, N. R., Molec. Microb. 2008, 69, 994-1007;
⁴Gillen, J. R.; A. J., in Mechanisms in Recombination, Grell, R. F., Ed. Plenum Press: New York, 1974; pp 123-136;
⁵Weige, J., Proc. Natl. Acad. Sci. USA 1966, 55, 1462-1466; and
⁶Appleyard, R. K., Genetics 1954, 39, 440-452.

TABLE 12
phage λ strains.

| Strain | Genotype^{a} | Source^{a} or ref. |
|---|---|---|
| 1081 | susJ6 b1453 cI857 χ⁺D123 | ^{1, 7} |
| 1082 | b1453 χ⁺D123 susR5 | ^{1, 7} |
| 1083 | susJ6 b1453 χ⁺76 cI857 | ^{1, 7} |
| 1084 | b1453 χ⁺76 susR5 | ^{1, 7} |
| DE3 | imm21 Δnin5 Sam7 P_{lacUV5} gene 1 | Novagen |
| | (T7 RNA polymerase) | |

Notes and References for Table 12:
^{a}b1453 is a deletion removing red, which encodes recombination proteins exo and beta, and gam, which encodes an inhibitor of RecBCD. These phage recombine by the E. coli RecBCD pathway.
¹Schultz, D. W.; Taylor, A. F.; Smith, G. R., J. Bacteriol. 1983, 155, 664-680;
⁷Stahl, F. W.; Stahl, M. M., Genetics 1977, 86, 715-725.

Phage T4 wild type and gene 2 amN51 mutant are as described in Schultz, D. W.; Taylor, A. F.; Smith, G. R., J. Bacteriol. 1983, 155, 664-680. A derivative bearing three adjacent nonsense mutations is described herein. Stocks of T4 phage were grown in strain V67, which lacks RecBCD and does not suppress the nonsense mutation(s) so that the phage particles contain DNA not protected by gene 2 protein.

Plasmids and Oligonucleotide primers. Plasmids are listed in Table 13, and oligonucleotides in Table 14.

TABLE 13
Plasmids.

| | Vector and | | Source or |
|---|---|---|---|
| Plasmid | insertion site | E. coli or H. pylori insert | ref. |
| pBR322 | | None | ⁸ |
| pETDuet-1 | | None | Novagen |
| pACYCDuet-1 | | None | Novagen |
| pSA21 | pBR322, | E. coli recB²¹CD | ⁹ |
| | BamHI | | |
| pMR3 | pBR322, | E. coli recBCD wild type | ² |
| | BamHI | | |
| pSA405 | pETDuet-1, | H. pylori addAB wild type | ³ |
| | NdeI PstI | | |
| pSA502 | pACYCDuet-1, | H. pylori recA wild type | ³ |
| | NcoI PstI | | |
| pSA520 | pBR322, HindIII | T4 gene 2 amN51 (W247*UAG) | This work |
| pSA524 | pBR322, HindIII | T4 gene 2 am149 (W247*UAG, | This work |
| | | A248*UAG, N249*UAA) | |
| pSA600 | pBR322 | None; bp 381-1624 deleted | This work |
| pSA607 | pSA600, EcoRI | E. coli recBD at BamHI, recC at EcoRI | This work |
| | BamHI | | |

References for Table 13:
⁸Bolivar, F.; Rodriguez, R. L.; Greene, P. J.; Betlach, M. C.; Heyneker, H. L.; Boyer, H. W.; Crosa, J. H.; Falkow ,S., Gene 1977, 2, 95-113;
⁹Amundsen, S. K.; Taylor, A. F.; Chaudhury, A. M.; Smith, G. R., Proc. Natl. Acad. Sci. USA 1986, 83, 5558-5562;
²Amundsen, S. K.; Taylor, A. F.; Reddy, M.; Smith, G. R., Genes Dev 2007, 21 (24), 3296-307; and
³Amundsen, S. K.; Fero, J.; Hansen, L. M.; Cromie, G. A.; Solnick, J. V.; Smith, G. R.; Salama, N. R., Molec. Microb. 2008, 69, 994-1007.

TABLE 14
Oligonucleotides.

| Oligo | |
|---|---|
| number | Nucleotide sequence |
| OL2636 | 5′ CATATGAAGCTTGTCAGTGTTTGCTGCAAATACTCCCC |
| | ATG 3′ (SEQ ID NO: 1) |
| OL2637 | 5′ CATATGAAGCTTCACCGTTCTCATTCACATGATAT |
| | AC 3′ (SEQ ID NO: 2) |
| OL2652 | 5′ |
| | TGAAATCGCCCCGAAAGACTAGTAGTAAGTTGTGTTGATG |
| | CCACTTCAGC 3′ |
| | (SEQ ID NO: 3) |
| OL2653 | 5′ |
| | GCTGAAGTGGCATCAACACAACTTACTACTAGTCTTTCGG |
| | GGCGATTTCA 3′ |
| | (SEQ ID NO: 4) |

Bacterial Growth Media.

Luria-Bertani (LB) broth contains 1.0% (w/v) Tryptone (Difco), 0.5% yeast extract (Difco), and 0.5% NaCl. LB agar is LB broth with 1.5% agar (Difco). Cation-adjusted Mueller-Hinton broth was purchased from Becton-Dickinson. TB contains 1.0% Tryptone and 0.5% NaCl; for phage λ crosses, 0.1% maltose was added. BBL top and bottom agar contain TB with 0.75% and 1% agar, respectively. BBL-YE is BBL bottom agar supplemented with 0.2% yeast extract. For growth of strains with plasmids, media were supplemented with ampicillin (100 μg/ml) or chloramphenicol (15 μg/ml). Cultures were grown at 37° C.

Purified RecBCD and AddAB Enzyme Assays.

Nuclease assays measured the formation of TCA-soluble radioactive material from phage T7 [³H] DNA (2 μg/ml; 6 μM nucleotides) substrate in a 20 min incubation at 37° C. (Eichler, D. C.; Lehman, I. R., J. Biol. Chem. 1977, 252, 499-503). AddAB assays were in 50 mM Tris-HCl (pH 8.5), 10 mM MgCl₂, polyvinylpyrrolidone (1 mg/ml), 1 mM DTT, and 50 μM ATP. RecBCD assays used the same condition but with 25 μM ATP. Compounds were diluted in DMSO and added to enzyme in assay buffer on ice; final DMSO concentration was 5.0% in each assay. DNA substrate was added, and after <5 min the reactions were started by transferring the samples to 37° C. Reactions were stopped by addition of calf thymus DNA to 0.2 mg/ml and TCA to 5%. After 10 min on ice, the mixtures were centrifuged for 5 min at 16,100×g, and the soluble radioactive material determined in a scintillation counter.

Helicase assays measured the formation of ss DNA from 5′ [³²P] pBR322 χ⁺F (or χ⁰ control) DNA (0.1 nM molecules) linearized by digestion with HindIII enzyme. AddAB assays were in 25 mM Tris-acetate (pH 7.5), 2.0 mM Mg(OAc)₂, 5 mM ATP, 1.5 μM SSB and used 1 nM enzyme. RecBCD assays used the same conditions but with 0.15 nM enzyme and without SSB. Compounds were added to the reaction mixture containing all the reagents except ATP; final DMSO concentration was 2.5% for each assay. Reactions were started by addition of ATP and were at 37° C. for 1 min (RecBCD) or 2 min (AddAB). Reactions were stopped by addition of ⅓ vol of stop buffer (2.5% SDS, 100 mM EDTA, 0.125% bromophenol blue, 0.125% xylene cyanol FF, and 10% Ficoll), and the products subjected to electrophoresis in a 1.25% agarose gel at 5 V/cm for 2.5 h in TAE buffer (40 mM Tris base, 20 mM acetic acid, 1 mM EDTA). Gels were dried under vacuum, and the products detected by autoradiography or analyzed with a Typhoon Trio Phosphorlmager (GE Lifesciences) and ImageQuant TL software (Amersham). With RecBCD, this assay also detects cutting of DNA at the Chi site χ⁺F, which produces a 1.46 kb fragment (Smith, G. R.; Kunes, S. M.; Schultz, D. W.; Taylor, A.; Triman, K. L., Cell 1981, 24, 429-436).

Genetic Assays.

E. coli Hfr recombination, phage λ recombination, and Chi hotspot activity assays were conducted as described by Schultz, D. W.; Taylor, A. F.; Smith, G. R., J. Bacteriol. 1983, 155, 664-680.

To test for inhibition of Hfr recombination, recipient cells were grown in LB to an optical density (OD) of 0.25 at 650 nm. Compound was added and incubation continued until the OD reached 0.5 (typically 30 to 45 min later). An aliquot was mixed with donor strain V1306 (Hfr P044) in the ratio of one donor cell per ten recipient cells. After 8 min, the mixture was diluted 1:50 into LB with compound. After 20 additional min, cells were vortexed to separate mating pairs, diluted, and plated to select recombinants. Viability of the cells was not significantly affected by compounds during this 1.5 h incubation.

To test for inhibition of phage λ recombination and Chi hotspot activity, E. coli strain V66 (recBCD⁺) was grown as above except in TB plus 0.1% maltose. Cells were infected with λphages at an MOI of 5 each; cross 1 was Δ 1081×λ 1082, and cross 2 was λ 1083×λ 1084. After 15 min, cells were diluted 1:100 into TB with compound at the same concentration, incubated at 37° C. for 90 min, and treated with chloroform. Phage were titered on strain 594 (sup⁺) for J⁺ R⁺ recombinants and on strain C600 (supE44) for total phage. Chi hotspot activity was measured as √(T1/C1)/(T2/C2), where T1/C1 is the ratio of turbid (c⁺) to clear (cl857) J⁺ R⁺ recombinants in cross 1 and T2/C2 is the same for cross 2 (Stahl, F. W.; Stahl, M. M., Genetics 1977, 86, 715-725).

Construction of T4 Gene 2 am149 Triple Nonsense Mutant Phage.

Phage T4 gene 2, including 851 bp 5′ and 842 bp 3′ of the ORF, was amplified from a lysate of phage T4 gene 2 amN51 by a PCR using oligonucleotides OL2636 and OL2637, Platinum Taq Polymerase (Invitrogen), and the manufacturer's suggested conditions. The product was purified on a QIAquick column (Qiagen), digested with HindIII (New England Biolabs), and ligated into HindIII-cleaved pBR322 to yield plasmid pSA520. The sequence of gene 2 in this plasmid was that of wild type except for 5′ TAG 3′ at codon 247 (5′ TGG 3′ in wild type). Two additional nonsense mutations were introduced into this gene at codons 248 (5′ GCG 3′→5′ TAG 3′) and 249 (5′ AAC 3′→5′ TAA 3′) using a QuikChange reaction (Stratagene-Agilent Technologies) and oligonucleotides OL2652 and OL2653 to yield plasmid pSA524. Strain V67 (recB21) transformed with this plasmid was grown in TB; about 1×10⁶ cells were embedded in BBL top agar on an LB agar plate, and about 1×10⁵ T4 wild-type phage spotted on this lawn. After overnight incubation at 37° C., phage were harvested, diluted, and plated on strain V67. About 100 small plaques, from a total of about 600 plaques, were transferred with toothpicks to a lawn of V67 and to a lawn of strain V66 (recB⁺). Phage that grew on V67 but not on V66, about 10% of the total tested, were plaque-purified, grown in V67, and confirmed to contain the expected triple non-sense mutations. This complex mutation is designated gene 2 am149.

Compound Screen in 96-Well Format.

A fresh overnight culture of strain V66 in LB was diluted 1:100 into LB broth with 0.1% DMSO and grown with aeration at 37° C. to an OD₆₅₀ of 0.05. Each well of a 96-well plate (Costar, Corning Inc.) was prepared by adding 10 μl of compound in 20% DMSO or the appropriate control (LB with 20% DMSO) and then 100 μl of the bacterial culture (containing about 2.5×10⁶ cells) or uninoculated medium was added. The OD₆₅₀ of each well was read on a VERSAmax microplate reader (Molecular Devices), and the plates were incubated without shaking at 37° C. After about 1 h the OD₆₅₀ of the cultures was approximately 0.1, and 10 μl of a phage suspension containing 5×10⁴ phage or 2.5 μg of chloramphenicol was added to the appropriate wells. The plates were incubated for approximately 20 h and the OD₆₅₀ determined.

AddAB (Strain V3065) Primary Assay.

All reagents were purchased from Sigma unless noted otherwise below. Prior to assay, strains V3065 (addAB⁺) and V3069 (vector control) were grown at 37° C. to an OD₆₀₀ of 0.05 (about 2.5×10⁷ cfu/mL). Three μL of assay buffer containing glycerol (0.1% v/v) and ampicillin (100 μg/mL) (Fisher) in Cation-adjusted Mueller Hinton Broth (Becton-Dickinson) were added to each well of a 1,536-well clear-bottom plate (Aurora, Nexus Biosystems). Sixty nL of test compound (12 μM final concentration), ciprofloxacin (0.95 μg/mL final concentration, as a control for complete inhibition), or DMSO alone (1.2% final concentration) were added to the appropriate wells; compounds and ciprofloxacin were in DMSO. One μL of strain V3065 (addAB⁺) or strain V3069 (vector control) was dispensed into the appropriate wells, and plates were incubated for 60 min at 37° C. One μL of phage T4 gene 2 am149 mutant was dispensed to the appropriate wells at a multiplicity of infection (MOI) of 0.02. Plates were centrifuged, and after 18 h of incubation at 37° C. the absorbance, as OD₆₀₀, was read on an Envision microplate reader (PerkinElmer). All data were normalized to that of the positive control (wells containing strain V3065, ciprofloxacin, and phage) and negative control (wells containing strain V3065, DMSO, and phage). This protocol was used for primary, secondary, and titration screening assays. The hit-cutoff used to qualify active compounds in the primary assay was calculated as the average percentage activity of all compounds tested plus three times their standard deviation (Hodder, P.; Cassaday, J.; Peltier, R.; Berry, K.; Inglese, J.; Feuston, B.; Culberson, C.; Bleicher, L.; Cosford, N. D.; Bayly, C.; Suto, C.; Varney, M.; Strulovici, B., Anal. Biochem. 2003, 313 (2), 246-54).

The secondary or confirmation assay used the same conditions as the primary screening assay, except that plates were assessed in triplicate and results for each compound were reported as the average percentage activity of the three measurements, plus or minus the associated standard deviation. For titration experiments, assay protocols were identical to those described above, except that compounds were prepared in 10 point, 1:3 serial dilutions starting at a nominal test concentration of 120 μM, and assessed in triplicate (Madoux, F.; Li, X.; Chase, P.; Zastrow, G.; Cameron, M. D.; Conkright, J. J.; Griffin, P. R.; Thacher, S.; Hodder, P., Mol. Pharmacol. 2008, 73 (6), 1776-84).

Bacterial Viability HTS Assay.

This assay was identical to the AddAB (strain V3065) screening assay, except that no phage was added to the wells. All data were normalized to that of the positive control (wells containing strain V3065 and ciprofloxacin) and negative control (wells containing strain V3065 and DMSO). This protocol was used for primary, secondary, and titration screening assays.

RecBCD (Strain V66) HTS Assay.

This assay was identical to the primary AddAB screening assay except that strains V66 (recBCD⁺) and V67 (recB21) replaced strains V3065 and V3069, respectively. All data were normalized to that of the positive control (wells containing strain V66, ciprofloxacin, and phage) and negative control (wells containing strain V66, DMSO, and phage). This protocol was used for secondary and titration screening assays.

TABLE 15
Protocol for AddAB or RedBCD Screening in 1536-well plates.

| Step | Operation | Condition | Comments |
|---|---|---|---|
| 1 | Medium dispensing | 3 μL/well | Medium is Cation Adjusted Mueller Hinton |
| | | | Broth (CAMHB) |
| 2 | Compound addition | 60 nL/well | Test concn, 12 μM; DMSO concn, 1.2% |
| 3 | Cell dispensing | 1 μL/well | Bacteria grown in CAMHB to 2.5 × 10⁷ cfu/mL |
| 4 | Incubation | 60 min at 37° C. | |
| 5 | Phage addition | 1 μL/well | Phage concn, 5 × 10⁵/mL; MOI, 0.02 |
| 6 | Incubation | 18 hr at 37° C. | |
| 7 | Optical Density | Read plate | OD₆₀₀ optimized absorbance read on |
| | determination | | PerkinElmer Envision |

Notes for Table 16: Left: Growth of strain V66 in the absence (black bars) or presence (gray bars) of phage T4 gene 2 triple nonsense mutant, as in FIG. 2, in the presence of the indicated compounds (100 μM). “RecBCD⁻” used strain V67 (recB21). Data are the mean of two wells; range is <5% of mean. Right: The frequency of His⁺ Str^{R} recombinants in matings between strains V66 (F⁻ recBCD⁺ hisG4 rpsL31) and V1306 (Hfr P044 rpsL⁺ his⁺) in the presence of compound is expressed as a fraction of that in the absence of compound (7.6 and 8.4% per viable Hfr cell in the two experiments for which data are shown).

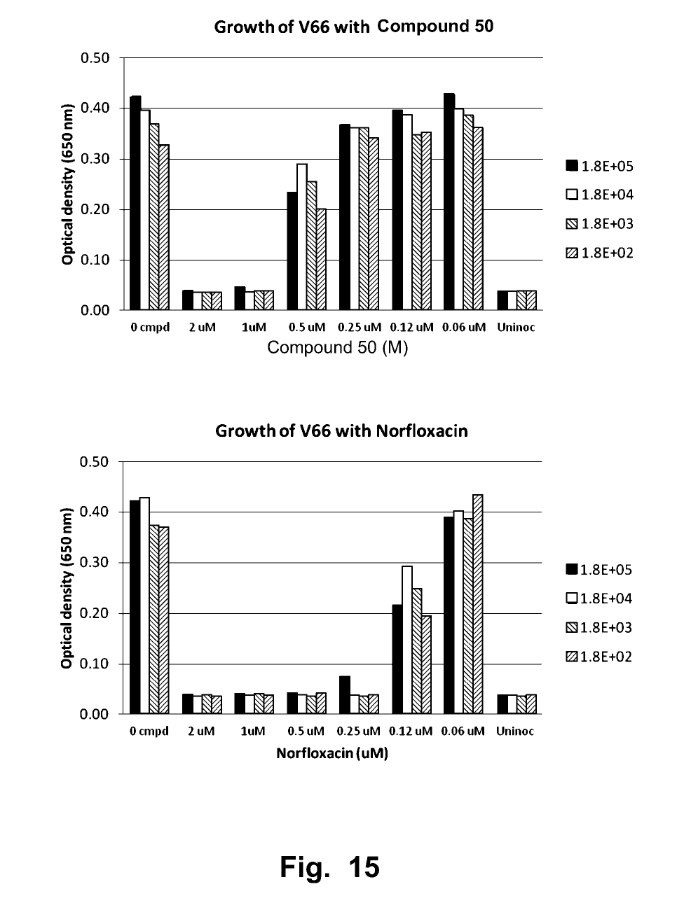

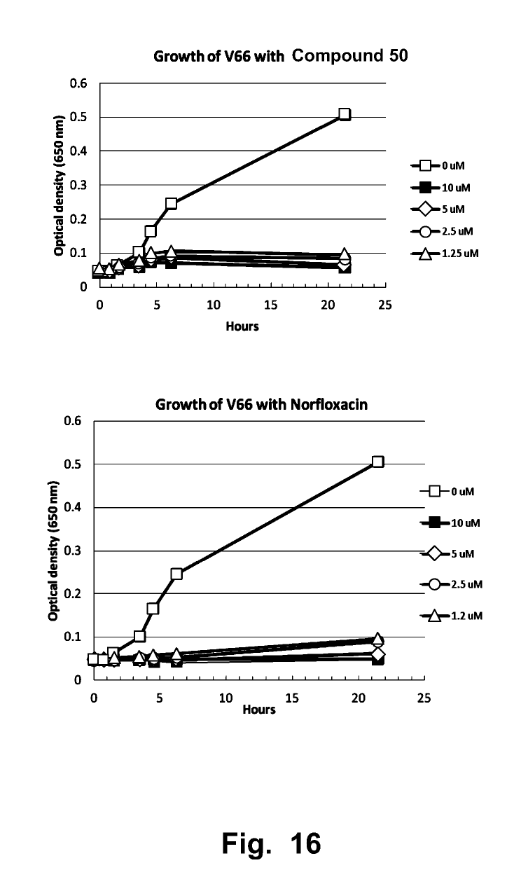

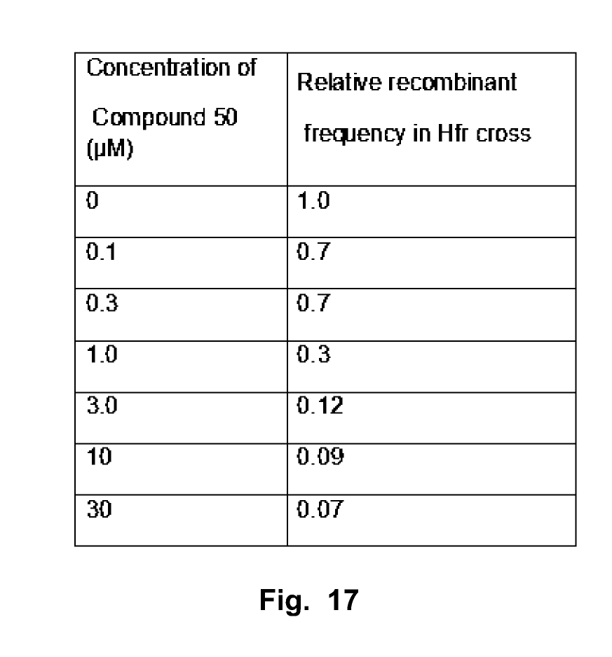

Further analysis of compound 50 is shown in FIG. 15-17. FIG. 15 shows the minimum concentration of compound 50 and norfloxacin required to inhibit the growth of E. coli strain V66 (recBCD⁺). FIG. 16 shows the inhibition of E. coli growth by Compound 50 or norfloxacin, and FIG. 17 shows that compound 50 inhibits E. coli recombination in an Hfr cross.

### Example 2

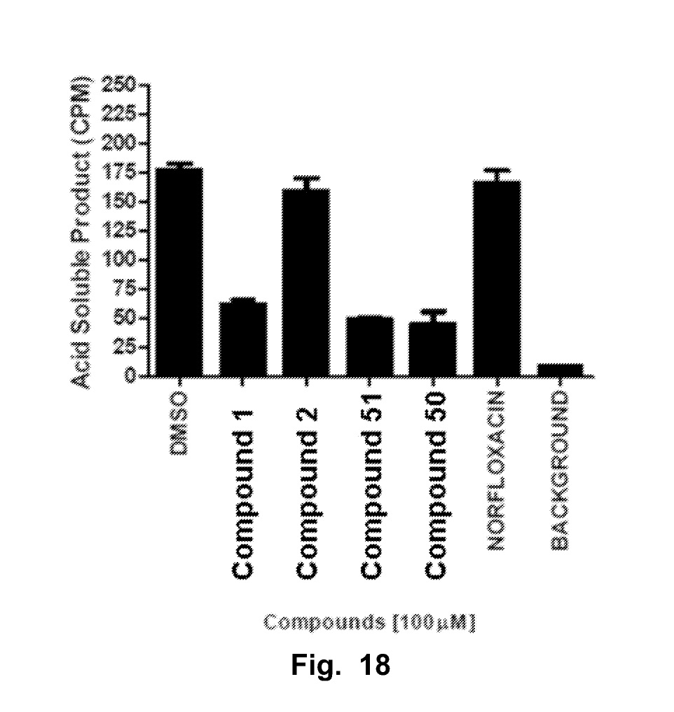

Compounds 1, 2, 50 and 51 were analyzed further. FIG. 18 shows their activity against E. coli RecBCD nuclease. In this experiment, the acid-soluble product formation is a measure of the nuclease activity of the RecBCD: more acid-soluble product indicates high nuclease activity. Compound 50 showed inhibition of both E. coli RecBCD and H. pylori AddAB nuclease activity at 100 μM and 50 μM (data not shown). FIG. 18 suggests that changes in the structure of the fluoroquinolone portion of the molecule does not affect helicase inhibition and that norfloxacin alone does not inhibit nuclease activity.

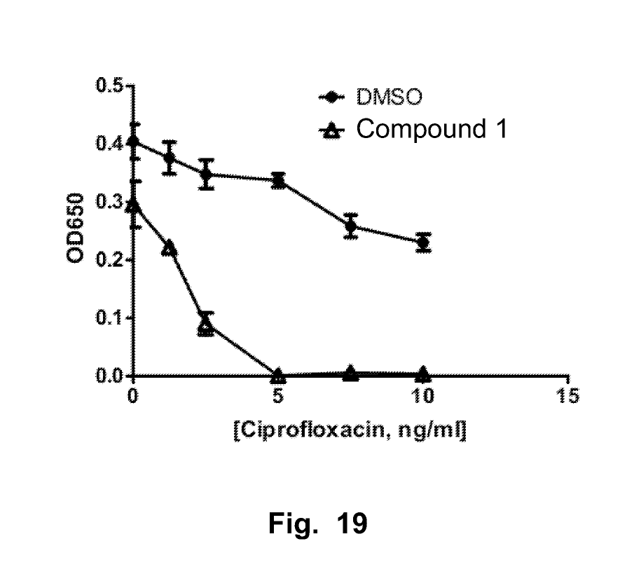

FIG. 19 shows the effect of compound 1 on the ciprofloxacin sensitization of an E. coli V66 wild type strain. In this assay, diluted bacteria grew overnight in the presence of Ciprofloxacin at the indicated concentration with 50 μM compound 1 or DMSO (1% final concentration). FIG. 19 suggests that inhibition of RecBCD enhances the antibacterial effects of fluoroquinolones such as ciprofloxacin. Compound 1 inhibits RecBCD but is a weak gyrase inhibitor. As can be seen from FIG. 19, bacteria were unable to grow in the presence of 5 ng/ml ciprofloxacin and 40 μM compound 1. The MIC for the inhibition of bacterial growth for compound 1 was 200 μM with a IC₅₀ of 78 μM for E. coli V66 (data not shown). When mixed with ciprofloxacin at concentrations (40 μM) that generally have little effect on bacterial growth, compound 1 sensitized wild-type E. coli to ciprofloxacin and decreased the MIC of ciprofloxacin in this strain 2- to 4-fold. When compared to the DMSO control, 40 μM compound 1 sensitized V66 E. coli cells to ciprofloxacin and enhanced the effect of ciprofloxacin. This suggests that inhibition of RecBCD helicase results in an improved antibacterial activity for fluoroquinolones, and that development of, for example, a ciprofloxacin version of compound 1 or compound 50, may lead to improved antibacterial drugs when compared to conventional quinolones.

FIG. 20 shows a dose response study of compound 1 in the inhibition of RecBCD helicase and Chi cutting activities. Compound 1 appears to act upon RecBCD in a biphasic manner. The enzyme is effectively inactive at concentrations starting around 40 μM, which correlates with the sensitization of E. coli V66 strain to sub-inhibitory concentrations of ciprofloxacin in the presence of 40 μM compound 1 as seen in FIG. 19.

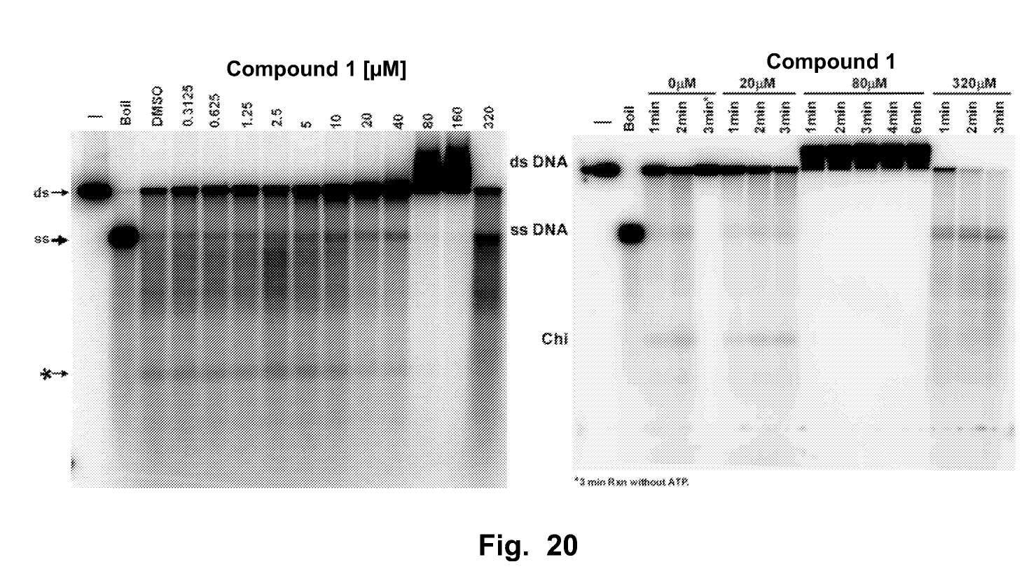

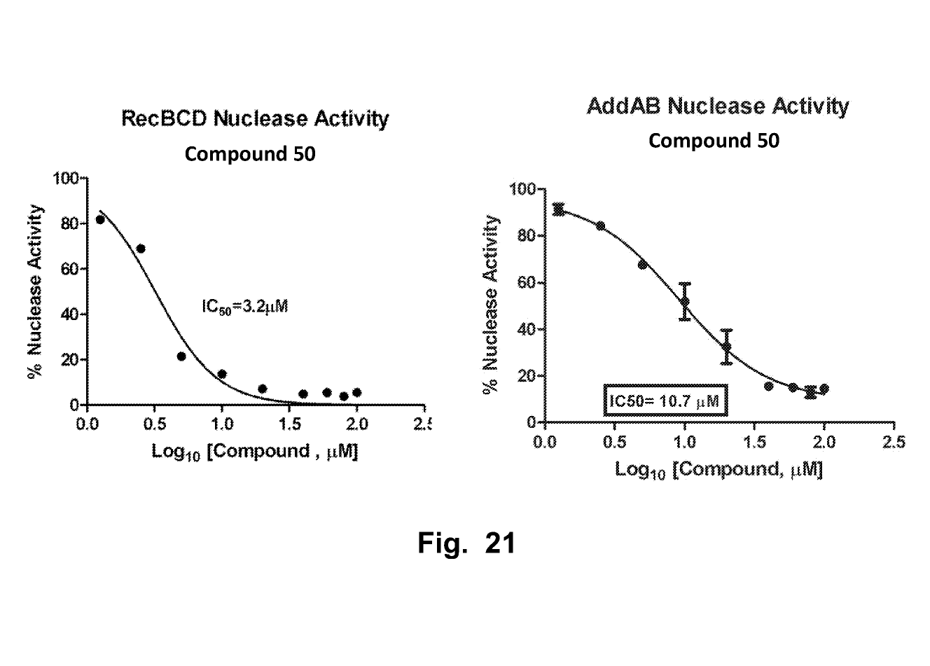

FIG. 21 shows a dose response study of compound 50 in the inhibition of E. coli RecBCD and H. pylori AddAB ds exonucleases.

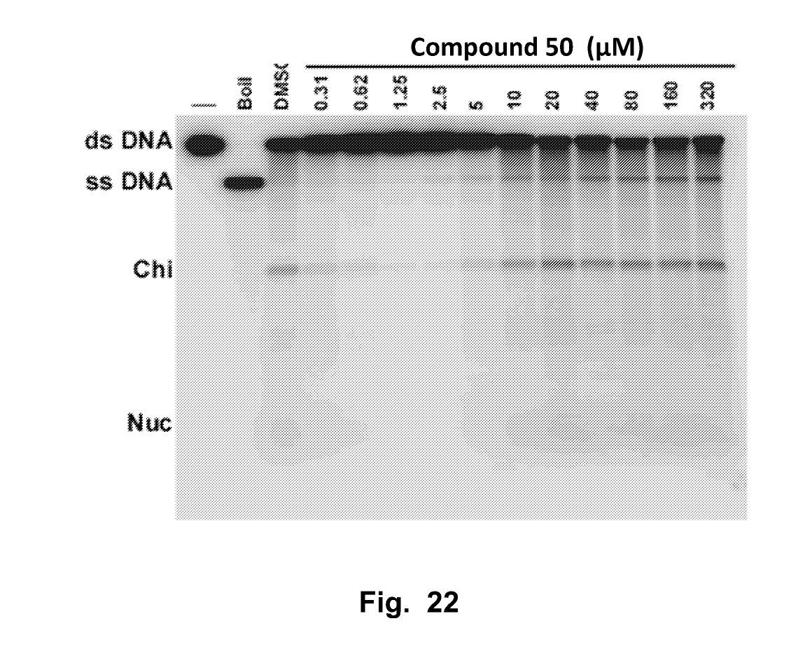

FIG. 22 shows a dose response study of the inhibition of E. coli RecBCD DNA unwinding and Chi cutting activities for compound 50. This gel result was similar to that of compound 3, which is a weak gyrase inhibitor but inhibits RecBCD. This suggests that changing the quinolone portion of the molecule, to make it a more potent gyrase inhibitor, will still retain potency of the compound against DNA helicase.

### Example 3

Several compounds as described herein were evaluated for their ability to inhibit bacterial DNA gyrase. Each of compounds 22, 30, 33, 35, 36, and 37 as described above were screened against E. coli gyrase to determine whether they are inhibitors of gyrase-catalysed supercoiling, and each of the compounds inhibited gyrase-catalyzed supercoiling at IC₅₀ values below 200 μM, with compounds 22, 33, and 37 exhibiting IC₅₀ values as low as 80 μM, and compound 35 exhibiting an IC₅₀ as low as 40 μM.

### Example 4

As shown in Table 17, compounds disclosed herein were assayed for their ability to inhibit E. coli RecBCD, H. pylori AddAB, M. smegmatis AddAB, and M. smegmatis RecBCD. The purified AddAB and RecBCD enzyme assays were performed as described in Example 1. The H. pylori AddAB, M. smegmatis AddAB and RecBCD, and M. tuberculosis AddAB enzymes were obtained from Seattle Structural Genomics Center for Infectious Disease (Seattle, Wash.).

TABLE 17
AddAB and RecBCD enzyme assays.

| | Laboratory | Nuclease IC₅₀, [μM] | | | |
|---|---|---|---|---|---|
| Com- | Compound | E. coli | H. pylori | M. smeg | M. smeg |
| pound | Name | RecBCD | AddAB | AddAB | RecBCD |
| 1 | 104 | 13 | 34 | — | — |
| 3 | 104-34 | 4.6 | 16 | 2.4 | 5.5 |
| 143 | 104-64 | 4.7 | 6.6 | 7 | 30 |
| 30 | 104-26 | 20 | 46 | — | — |
| 51 | Norf-104 | 26 | 79 | — | — |
| 50 | Norf-34 | 3.2 | 10.7 | 31 | 4.5 |
| 144 | Norf-64 | 3.7 | 16 | 3.2 | 37 |
| 57 | Cipro-34 | 10 | 12 | 23 | 6 |
| 150 | Cipro-64 | 3 | — | 6.6 | 4.9 |
| 64 | Gati-26 | 1.2 | 16 | — | — |
| 58 | Gati-34 | 1.8 | 3.8 | 3.4 | 2.1 |
| 145 | Gati-64 | 1.2 | — | 4.5 | 12 |
| 151 | Moxi-34 | 0.7 | 0.8 | 3.8 | 10 |
| 152 | Moxi-64 | — | — | 1.8 | 5.4 |
| 148 | Sara-34 | 0.65 | 0.8 | 3.3 | 7.2 |
| 149 | Sara-64 | 0.58 | 0.7 | 0.6 | 2.6 |
| 146 | Lome-34 | 6.3 | 8.4 | 1.2 | 20 |
| 147 | Lome-64 | — | — | 3.1 | 12 |

With reference to Table 17, compounds 3, 26, 50, 148, and 151, among others, resulted in inhibition of the RecBCD and AddAB enzymes tested. More particularly, compound 3 had an IC₅₀ of 4.6 μM for E. coli RecBCD, an IC₅₀ of 16 μM for H. pylori AddAB, an IC₅₀ of 2.4 μM for M. smegmatis AddAB, and an IC₅₀ of 5.5 μM for M. smegmatis RecBCD. Compound 151 showed an IC₅₀ of 0.7 μM for E. coli RecBCD, an IC₅₀ of 0.8 μM for H. pylori AddAB, an IC₅₀ of 3.8 μM for M. smegmatis AddAB, and an IC₅₀ of 10 μM for M. smegmatis RecBCD.

### Example 5

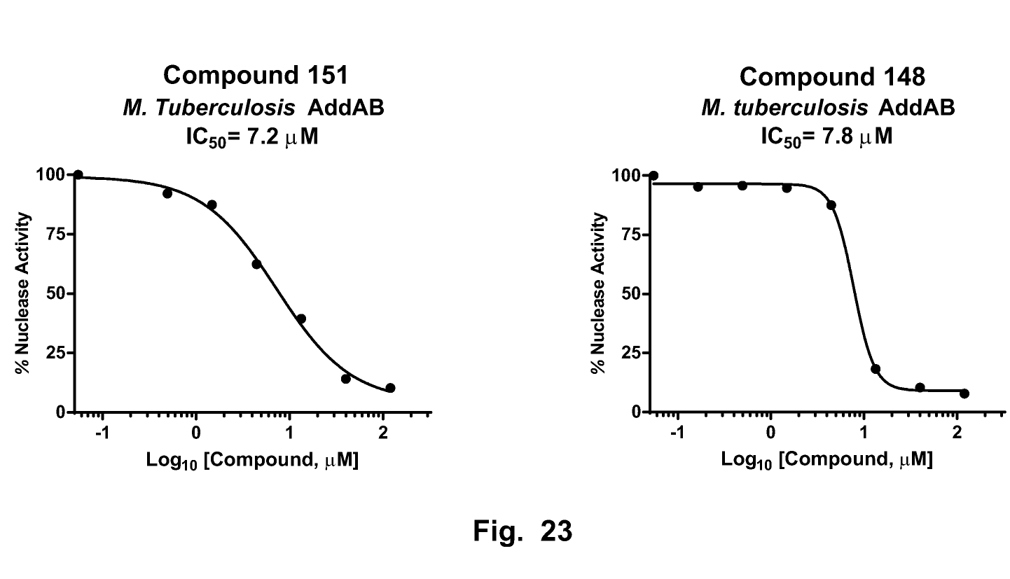

Compounds 151 and 148 as described herein were evaluated for their ability to inhibit M. tuberculosis AddAB enzyme. The purified M. tuberculosis AddAB enzyme ds exonuclease assay was performed as described in Example 1. As shown by FIG. 23, both compounds inhibited M. tuberculosis AddAB, with compound 151 having an IC₅₀ of 7.2 μM and compound 148 having an IC₅₀ of 7.8 μM.

### Example 6

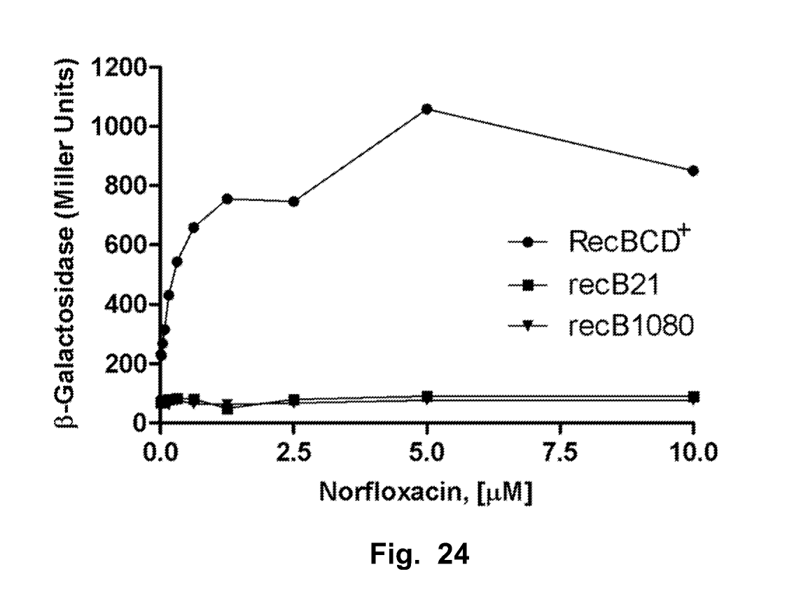

An E. coli precA::lacZ reporter assay was used for the measurement of SOS induction by norfloxacin and its dependence on RecBCD nuclease activity (FIG. 24). E. coli precA::lacZ strains were GE94 (Weisemann et al., 1984) or recB21 (null) or recB1080 (nuclease-defective) mutant derivatives. Strains were grown at 37° C. in LB broth to OD₆₅₀≈0.4, norfloxacin was added to the indicated concentration, and incubation continued 60 min, at which time the cultures were assayed for beta-galactosidase according to Weisemann et al. (1984). All cultures contained 2% DMSO, final concentration.

Norfloxacin and related fluoroquinolone antibiotics kill bacteria by damaging their DNA. These antibiotics cause DNA double strand breaks in bacterial chromosomes by inhibiting DNA gyrase. When there is a DNA break, RecBCD enzyme acts on the break and RecBCD activity induces a gene network to fix the DNA damage. This overall pathway is called the SOS response, and induction of this pathway is called SOS induction. SOS response increases the survival of the bacteria when there is damage to their DNA in the presence of DNA damaging agents such as norfloxacin or hydrogen peroxide. Additionally, this repair pathway is very error prone and causes mutations that increase the chance of bacteria to develop resistance to antibiotics.

This network is activated by RecBCD enzyme and activation of this response can be measured by the expression of a reporter enzyme such as beta-galactosidase enyzme that is placed under the control of SOS inducible recA promoter. Increase in beta-galactosidase enzyme means increase in expression of SOS response genes. This way the activation of SOS response can be measured.

As can be seen in FIG. 24, when RecBCD enzyme is inactive as in null mutant (recB21) or nuclease mutant (recB1080), bacteria can't activate the SOS response in the presence of norfloxacin, resulting in no increase in beta-galactosidase expression. However, bacteria with fully functional RecBCD (RecBCD+) show a many-fold increase in SOS response in the presence of norfloxacin, as measured by increase in beta-galactosidase activity. Therefore, treatment with the antibiotic norfloxacin activates the SOS response in a RecBCD activity-dependent manner.

### Example 7

An E. coli precA::lacZ reporter assay was used for the measurement of RecBCD-dependant SOS induction with or without compound 151. E. coli precA::lacZ strains were GE94 (Weisemann et al., 1984) or a recB21 mutant derivative. Strains were grown at 37° C. in LB broth to OD₆₅₀≈0.4, the DNA-damaging agent H₂O₂ (hydrogen peroxide) was added to the indicated concentration with or without compound 151 (1 μM), and incubation continued 60 min, at which time the cultures were assayed for beta-galactosidase activity according to Weisemann et al. (1984). All cultures contained 2% DMSO, final concentration.

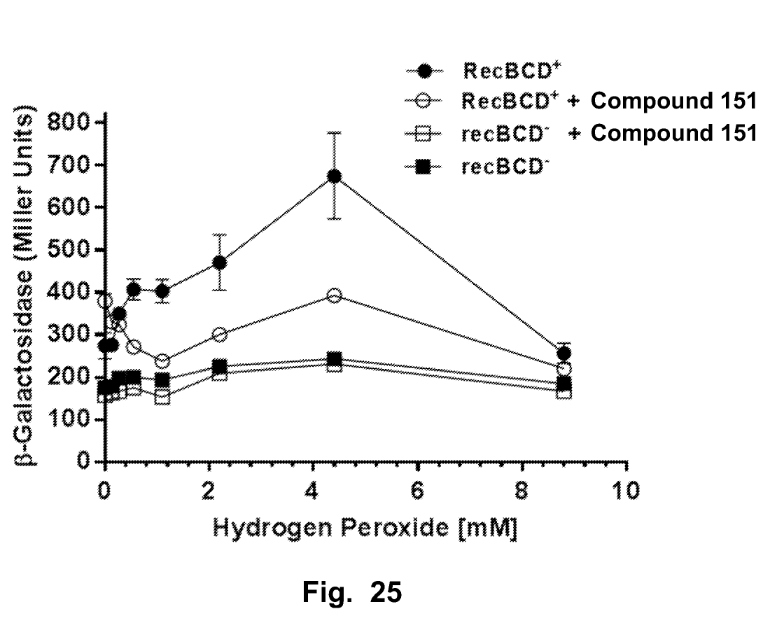

As shown in FIG. 25, in the presence of hydrogen peroxide, bacteria with fully functional RecBCD (RecBCD⁺) treated with compound 151 (RecBCD⁺+Compound 151) induced SOS response (empty circles) much less than the RecBCD⁺ bacteria without compound 151 (filled circles). Bacteria without active RecBCD (squares with recBCD⁻) were controls and showed no appreciable increase in beta-galactosidase activity. Accordingly, treatment the RecBCD inhibitor compound 151 blocks RecBCD-dependent activation of the SOS response.

### Example 8

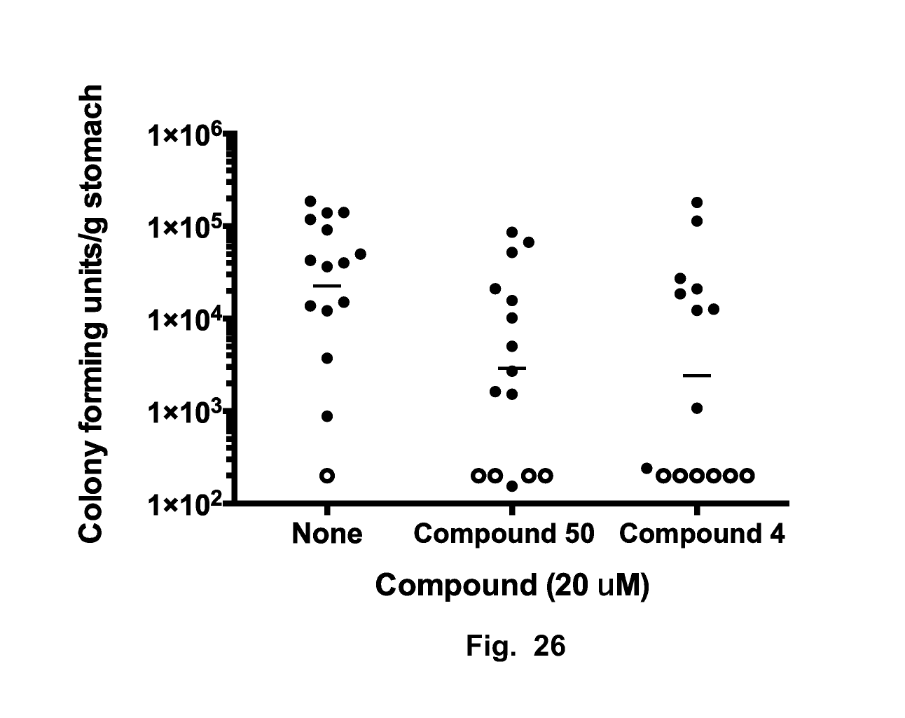

As shown by FIG. 26, AddAB inhibitors compound 50 and compound 4 impair the ability of Helicobacter pylori to colonize the stomach of mice. Mice were infected with 2.5×10⁷ cfu of H. pylori by oral gavage in the presence or absence of 20 μM compound 50 or compound 4 in methylcellulose. The same dose of compound in methylcellulose or methylcellulose alone was administered by oral gavage daily for 5 days. The stomachs were removed on day 7, processed by mechanical disruption, and the number of H. pylori per g stomach determined by plating on Columbia blood agar. For FIG. 26, each circle represents a single mouse; open circles are placed at the limit of detection for the conditions of culture and represent mice from which no H. pylori were recovered; the black horizontal line indicates the geometric mean colonization of each group.

### Example 9

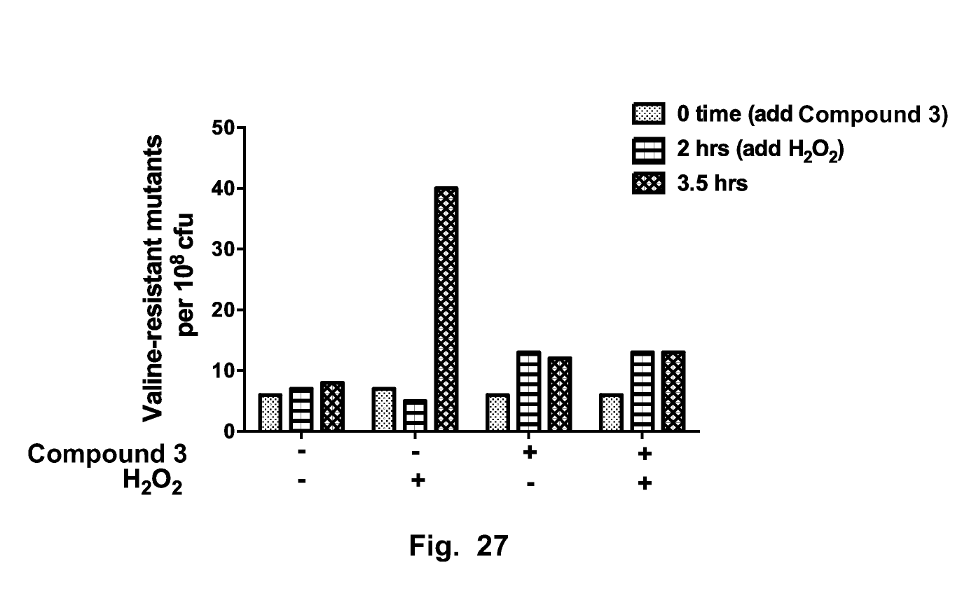

As shown by FIG. 27, RecBCD inhibitor compound 3 reduces the frequency of H₂O₂-induced mutation in E. coli. H₂O₂ and other reactive oxygen species damage DNA and, in a RecBCD-dependent manner, induce the SOS pathway which includes mutagenic DNA polymerases. E. coli strain V66 (recBCD⁺ valine-sensitive) was grown, as indicated, in the presence or absence of 25 μM compound 3 for 2 hr before the addition of 2 mM H₂O₂. The frequency of valine-resistant mutants in the culture at the times indicated was determined by plating on minimal media containing 100 ug/ml valine.

### Example 10

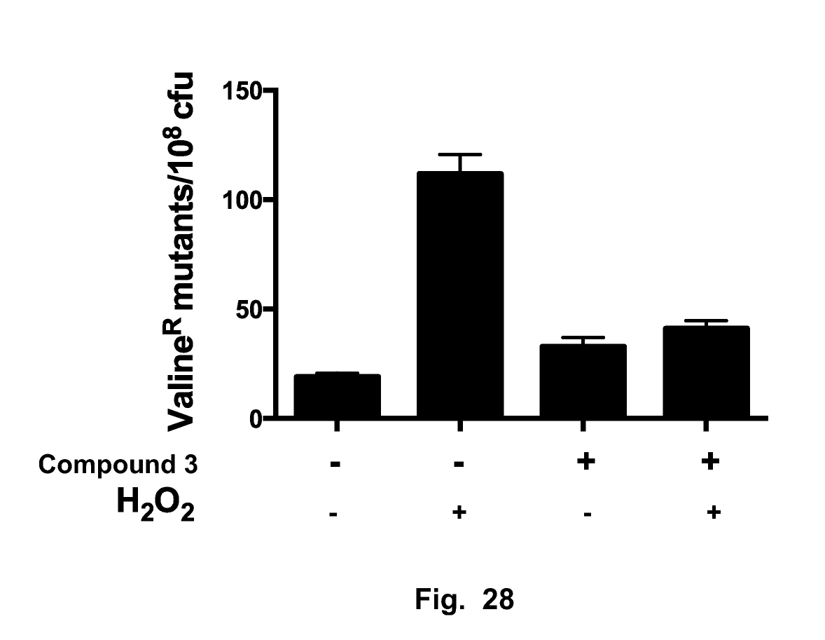

FIG. 28 shows that compound 3, a RecBCD inhibitor, reduces the frequency of H₂O₂-induced mutation to valine-resistance (valine^{R}) in E. coli. H₂O₂ and other reactive oxygen species damage DNA and, in a RecBCD-dependent manner, induce the SOS pathway, which includes mutagenic DNA polymerases. Strain V66 (recBCD⁺ valine-sensitive) was grown, as indicated, in the presence or absence of 25 μM compound 3 for 1 hr before the addition of 2 mM H₂O₂. The frequency of valine^{R} mutants in the culture was determined 1 hr later by plating on minimal media containing 100 ug/ml valine. The mean and standard error of the mean are shown for 16 separate cultures.

### Example 11

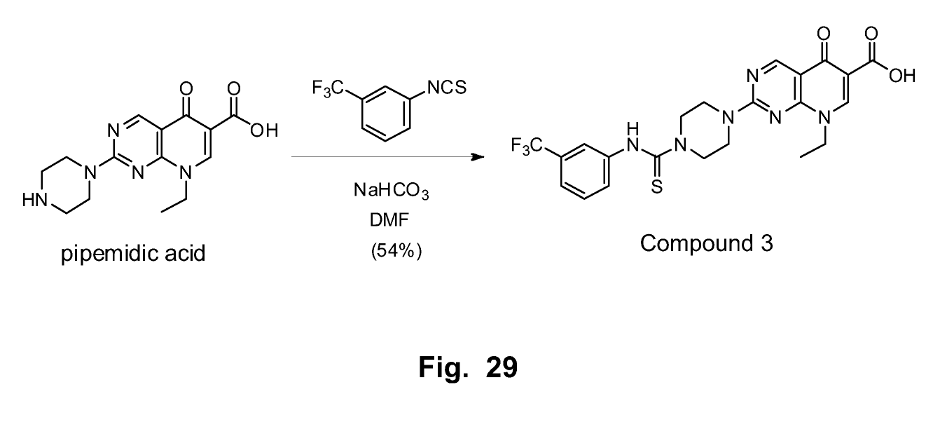

Compounds disclosed herein may be synthesized according to a single step. As shown in FIG. 29, compound 3 is synthesized in a straightforward fashion in a single step using commercially available reagents. Briefly, to an oven dried 100 mL round bottom flask equipped with a magnetic stir bar was added pipemidic acid (0.250 g; 0.824 mmol), 3(trifluoromethyl)phenyl isothiocyanate (0.167 g; 0.823 mmol), and sodium bicarbonate (0.083 g; 0.988 mmol), and the flask was flushed with Argon for 10 minutes. Dry N,N-dimethylformamide was added (40 mL) and the flask was once again flushed with Argon. The reaction mixture was allowed to stir for 15 hours at room temperature. After confirming the completion of reaction by TLC and LC-MS, the reaction was quenched by addition of 25 mL of a saturated NH₄Cl solution. The contents were transferred to a separatory funnel and extracted with ethyl acetate (3×25 mL). The organic extracts were combined and washed with water (3×25 mL), brine (1×25 mL), and dried over Na₂SO₄. Finally the solvents were evaporated on a rotary evaporator to give a crude solid. The crude material was then purified by flash chromatography (10% MeOH in CH₂Cl₂) to give 0.225 g (54% yield) of compound 3.

1. A compound which inhibits AddAB and/or RecBCD, the compound comprising an active compound according to one of Formulas I-V: 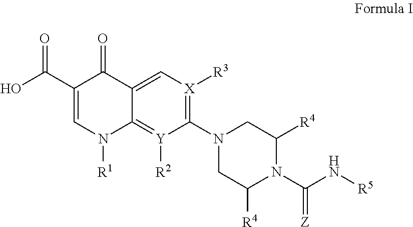
wherein R¹ is alkyl, aryl, or cycloalkyl;
R² is H, alkoxyl or halogen;
R³ is H or halogen;
R⁴ is H or alkyl;
R⁵ is selected from at least one of the following: alkyl, alkenyl, aryl, alkyl aryl, —CO-aryl, —CO-alkyl aryl, cycloalkyl, heteroaryl, and —CO-heteroaryl, any of which may be optionally substituted with a substituent selected from at least one of the following: alkyl, haloalkyl, alkoxy, methylenedioxy, halogen, ethylenedioxy, and nitro;
X and Y are independently C or N; and
Z is O or S: 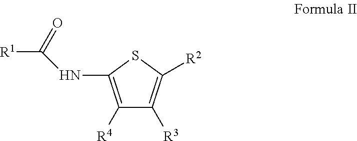
wherein R¹ is aryl, cycloalkenyl, heteroaryl, optionally substituted with a substituent selected from at least one of the following: alkyl, aryl, nitro, —COOH, thioalkyl, thioalkylaryl and halogen;
R² is H or alkyl;
R³ is H, alkyl, or aryl, each of which may be optionally substituted with an alkyl group, and wherein R² and R³ together may be connected to form a cycloalkyl or heterocyclic group, which may be optionally substituted with an alkyl group; and
R₄ is CN, —COO-alkyl, —CO—NH₂, —CO—NH-alkyl, —CO—NH-heterocyclyl, —CO—NH-alkyl-heterocyclyl, or NH₂: 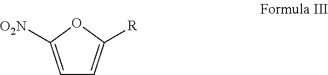
wherein R is selected from at least one of the following: —CO—O-alkyl heteroaryl, —CO—NH-heteroaryl, alkenyl heteroaryl, —CO—O-alkyl-CO—NH-heteroaryl, —CO—NH-aryl, and —CO—NH-alkyl aryl, any of which may be optionally substituted with a substituent selected from at least one of the following: C═O, N—CO-alkyl, CN, alkyl, —CONH₂, heterocyclyl or —NH—CO-haloaryl: 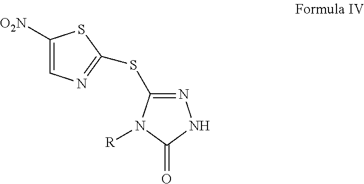
wherein R is an alkyl or alkenyl group: or 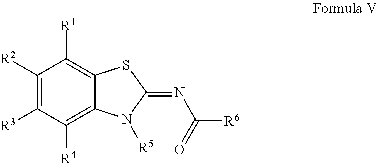
wherein R¹ is H;
R² is H, halo, alkyl, CONH-alkyl, nitro, CO₂-alkyl, SO₂-alkyl or SO₂NH₂;
R³ is H;
R⁴ is H, halo, alkyl, or alkoxy;
R⁵ is alkyl, alkenyl, alkynyl, alkyl alkoxy, or alkyl-CO-alkoxy; and
R⁶ is aryl, alkyl aryl, alkenyl aryl, alkenyl heteroaryl, alkyl-SO₂-aryl, alkyl-O-aryl, aryl-SO₂-heterocyclyl, heteroaryl, heterocyclyl, cycloalkyl, diphenyl or heterocycloalkenyl, any of which may be optionally substituted with a substituent selected from at least one of the following: nitro, halo, alkyl, alkoxy, aryl, —CO, —CO₂-alkyl, CO-substituted heterocyclyl, methylenedioxy, SO₂-alkyl, or halophenyl-substituted heteroaryl.

2. A compound according to claim 1, wherein the compound exhibits an IC₅₀ of less than 100 μM against a bacterial DNA helicase.

3. A compound according to claim 2, wherein the bacterial DNA helicase is a helicase selected from an AddAB helicase and a RecBCD helicase.

4. A compound according to claim 1, wherein the compound exhibits an IC₅₀ of less than 50 μM against a bacterial DNA helicase.

5. A compound according to claim 1, wherein the compound is selected from any of compounds 1-160.

6. A pharmaceutical composition for the treatment of a microbial, bacterial or fungal infection in a subject, the pharmaceutical composition comprising one or more compounds according to claim 1 and a pharmaceutically acceptable carrier or excipient.

7. A compound which inhibits a bacterial DNA helicase, nuclease, or helicase-nuclease complex selected from an AddAB helicase-nuclease and a RecBCD helicase-nuclease, the compound comprising an active compound according to Formula Ib: 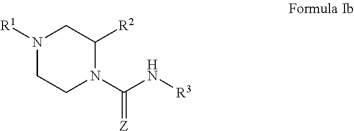
wherein R¹ is selected from at least one of the following: alkyl, alkenyl, aryl, alkyl aryl, cycloalkyl, heteroaryl, alkyl heteroaryl, heterocyclyl, and heterocyclyl alkyl, any of which may be optionally substituted;
R² is H or alkyl;
R³ is selected from at least one of the following: alkyl, alkenyl, aryl, alkyl aryl, —CO-aryl, —CO-alkyl aryl, cycloalkyl, heteroaryl, and —CO-heteroaryl, any of which may be optionally substituted with a substituent selected from at least one of the following: alkyl, haloalkyl, alkoxy, methylenedioxy, halogen, ethylenedioxy, and nitro; and
Z is O or S.

8. A compound according to claim 7,
wherein R¹ is selected from a compound according to Formula Ic 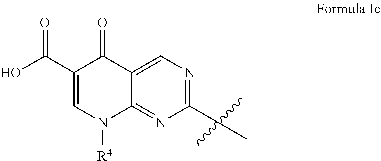
and R² is H, Z is S, R⁴ is alkyl and R³ is phenyl substituted with a haloalkyl group.

9. A compound according to claim 8, wherein R³ is phenyl substituted with a CF₃ group positioned in one of the ortho, para, and meta positions.

10. A compound according to claim 9, wherein the compound is selected from one of Compound 1, Compound 3, Compound 30, and Compound 143.

11. A compound according to claim 7, wherein R¹ is selected from a compound according to Formula Id 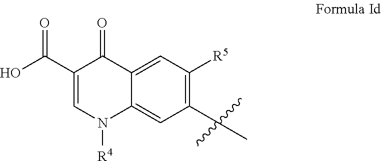
and R² is H, Z is S, R³ is phenyl substituted with a CF₃ group, R⁴ is alkyl, and R⁵ is fluorine.

12. A compound according to claim 11, wherein R³ is phenyl substituted with a CF₃ group positioned in one of the ortho, para, or meta positions.

13. A compound according to claim 12, wherein the compound is selected from one of Compound 50, Compound 51, Compound 144, Compound 145, Compound 146, Compound 147, Compound 148, Compound 149, and Compound 150.

14. A compound according to claim 7, wherein the compound exhibits an IC₅₀ of less than 100 μM against a bacterial DNA helicase, nuclease, or helicase-nuclease complex.

15. A compound according to claim 7, wherein the compound exhibits an IC₅₀ of less than 50 μM against a bacterial DNA helicase, nuclease, or helicase-nuclease complex.

16. (canceled)

17. A method of identifying an inhibitor of RecBCD activity, the method comprising:
testing a candidate compound for inhibition of RecBCD activity in a bacterial strain expressing an active RecBCD enzyme (recBCD⁺) in the presence of a T4 gene 2 mutant phage, wherein a lack of growth of the bacterial strain is indicative of the inhibition of RecBCD activity by the candidate compound.
